(19) United States
(12) Patent Application Publication
(10) Pub. No.: US 2007/0265278 A1
(43) Pub. Date: Nov. 15, 2007

(54) 2-PHENYL-INDOLES AS PROSTAGLANDIN D2 RECEPTOR ANTAGONISTS

(75) Inventors: Keith J. HARRIS, Chester, NJ (US); Hans-Jochen Lang, Hofheim-Wallau (DE); Rose M. Mathew, Ringoes, NJ (US); Stephen J. Shimshock, Hillsborough, NJ (US); Thaddeus R. Nieduzak, Bridgewater, NJ (US); Sharon Jackson, Whitehouse Station, NJ (US); Zhaoxia Yang, Whitehouse Station, NJ (US); Kenneth J. Bordeau, Kintnersville, PA (US)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: Aventis Pharmaceuticals Inc., Bridgewater, NJ (US)

(21) Appl. No.: 11/782,890

(22) Filed: Jul. 25, 2007

Related U.S. Application Data
(63) Continuation of application No. PCT/US06/02736, filed on Jan. 25, 2006.
(60) Provisional application No. 60/647,307, filed on Jan. 26, 2005.

(51) Int. Cl. A61K 31/404; A61K 31/4245; A61K 31/435; A61K 31/4453; A61K 31/47; A61K 31/495; A61P 1/04; A61P 37/08; A61P 9/00; C07D 209/04; C07D 271/113; C07D 401/12

(52) U.S. Cl. 514/255.04; 514/290; 514/311; 514/317; 514/323; 514/364; 514/419; 546/201; 548/144; 548/491

(57) ABSTRACT

The present invention is directed to a compound of Formula (XVI): (please replace Formula (I) with Formula (XVI) as shown below) 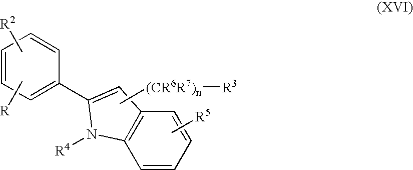
wherein R, R², R³, R⁴, R⁵, R⁶, R⁷ and n are as defined herein, or a pharmaceutically acceptable salt, hydrate, or solvate thereof, a pharmaceutically acceptable prodrug thereof, or a pharmaceutically acceptable salt, hydrate or solvate of the prodrug, a pharmaceutical composition comprising a pharmaceutically effective amount of one or more compounds according to Formula (XVI) in admixture with a pharmaceutically acceptable carrier, a method of treating a patient suffering from a PGD2-mediated disorder including, but not limited to, allergic disease (such as allergic rhinitis, allergic conjunctivitis, atopic dermatitis, bronchial asthma and food allergy), systemic mastocytosis, disorders accompanied by systemic mast cell activation, anaphylaxis shock, bronchoconstriction, bronchitis, urticaria, eczema, diseases accompanied by itch (such as atopic dermatitis and urticaria), diseases (such as cataract, retinal detachment, inflammation, infection and sleeping disorders) which are generated secondarily as a result of behavior accompanied by itch (such as scratching and beating), inflammation, chronic obstructive pulmonary diseases, ischemic reperfusion injury, cerebrovascular accident, chronic rheumatoid arthritis, pleurisy, ulcerative colitis and the like by administering to said patient a pharmaceutically effective amount of a compound according to Formula (XVI).

This application is a continuation of International Application No. PCT/US2006/002736, filed Jan. 25, 2006, which claims the benefit of Provisional Application No. 60/647,307, filed Jan. 26, 2005.

### FIELD OF THE INVENTION

The present invention is directed to 2-phenyl-indole compounds, their preparation, pharmaceutical compositions containing these compounds, and their pharmaceutical use in the treatment of disease states capable of being modulated by the inhibition of the prostaglandin D2 receptor.

### BACKGROUND OF THE INVENTION

Local allergen challenge in patients with allergic rhinitis, bronchial asthma, allergic conjunctivitis and atopic dermatitis has been shown to result in rapid elevation of prostaglandin D2 “(PGD2)” levels in nasal and bronchial lavage fluids, tears and skin chamber fluids. PGD2 has many inflammatory actions, such as increasing vascular permeability in the conjunctiva and skin, increasing nasal airway resistance, airway narrowing and eosinophil infiltration into the conjunctiva and trachea.

PGD2 is the major cyclooxygenase product of arachidonic acid produced from mast cells on immunological challenge [Lewis, R A, Soter N A, Diamond P T, Austen K F, Oates J A, Roberts L J II, prostaglandin D2 generation after activation of rat and human mast cells with anti-IgE, J. Immunol. 129, 1627-1631, 1982]. Activated mast cells, a major source of PGD2, are one of the key players in driving the allergic response in conditions such as asthma, allergic rhinitis, allergic conjunctivitis, allergic dermatitis and other diseases [Brightling C E, Bradding P, Pavord I D, Wardlaw A J, New Insights into the role of the mast cell in asthma, Clin Exp Allergy 33, 550-556, 2003].

Many of the actions of PGD2 are mediated through its action on the D-type prostaglandin (“DP”) receptor, a G protein-coupled receptor expressed on epithelium and smooth muscle.

In asthma, the respiratory epithelium has long been recognized as a key source of inflammatory cytokines and chemokines that drive the progression of the disease [Holgate S, Lackie P, Wilson S, Roche W, Davies D, Bronchial Epithelium as a Key Regulator of Airway Allergen Sensitization and Remodeling in Asthma, Am J Respir Crit Care Med. 162, 113-117, 2000]. In an experimental murine model of asthma, the DP receptor is dramatically up-regulated on airway epithelium on antigen challenge [Matsuoka T, Hirata M, Tanaka H, Takahashi Y, Murata T, Kabashima K, Sugimoto Y, Kobayashi T, Ushikubi F, Aze Y, Eguchi N, Urade Y, Yoshida N, Kimura K, Mizoguchi A, Honda Y, Nagai H, Narumiya S, prostaglandin D2 as a mediator of allergic asthma, Science 287, 2013-2017, 2000]. In knockout mice, lacking the DP receptor, there is a marked reduction in airway hypereactivity and chronic inflammation [Matsuoka T, Hirata M, Tanaka H, Takahashi Y, Murata T, Kabashima K, Sugimoto Y, Kobayashi T, Ushikubi F, Aze Y, Eguchi N, Urade Y, Yoshida N, Kimura K, Mizoguchi A, Honda Y, Nagai H, Narumiya S, Prostaglandin D2 as a mediator of allergic asthma, Science 287, 2013-2017, 2000]; two of the cardinal features of human asthma.

The DP receptor is also thought to be involved in human allergic rhinitis, a frequent allergic disease that is characterized by the symptoms of sneezing, itching, rhinorea and nasal congestion. Local administration of PGD2 to the nose causes a dose dependent increase in nasal congestion [Doyle W J, Boehm S, Skoner D P, Physiologic responses to intranasal dose-response challenges with histamine, methacholine, bradykinin, and prostaglandin in adult volunteers with and without nasal allergy, J Allergy Clin Immunol. 86(6 Pt 1), 924-35, 1990].

DP receptor antagonists have been shown to reduce airway inflammation in a guinea pig experimental asthma model [Arimura A, Yasui K, Kishino J, Asanuma F, Hasegawa H, Kakudo S, Ohtani M, Arita H (2001), Prevention of allergic inflammation by a novel prostaglandin receptor antagonist, S-5751, J Pharmacol Exp Ther. 298(2), 411-9, 2001]. PGD2, therefore, appears to act on the DP receptor and plays an important role in elicitation of certain key features of allergic asthma.

DP antagonists have been shown to be effective at alleviating the symptoms of allergic rhinitis in multiple species, and more specifically have been shown to inhibit the antigen-induced nasal congestion, the most manifest symptom of allergic rhinitis [Jones, T. R., Savoie, C., Robichaud, A., Sturino, C., Scheigetz, J., Lachance, N., Roy, B., Boyd, M., Abraham, W., Studies with a DP receptor antagonist in sheep and guinea pig models of allergic rhinitis, Am. J. Resp. Crit. Care Med. 167, A218, 2003; and Arimura A, Yasui K, Kishino J, Asanuma F, Hasegawa H, Kakudo S, Ohtani M, Arita H Prevention of allergic inflammation by a novel prostaglandin receptor antagonist, S-5751. J Pharmacol Exp Ther. 298(2), 411-9, 2001].

DP antagonists are also effective in experimental models of allergic conjunctivitis and allergic dermatitis [Arimura A, Yasui K, Kishino J, Asanuma F, Hasegawa H, Kakudo S, Ohtani M, Arita H, Prevention of allergic inflammation by a novel prostaglandin receptor antagonist, S-5751. J Pharmacol Exp Ther. 298(2), 411-9, 2001; and Torisu K, Kobayashi K, Iwahashi M, Nakai Y, Onoda T, Nagase T, Sugimoto I, Okada Y, Matsumoto R, Nanbu F, Ohuchida S, Nakai H, Toda M, Discovery of a new class of potent, selective, and orally active prostaglandin D₂ receptor antagonists, Bioorg. & Med. Chem. 12, 5361-5378, 2004].

### SUMMARY OF THE INVENTION

The present invention is directed to a compound of Formula (XVI): 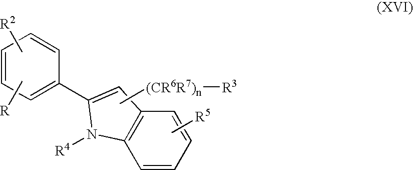
wherein:
R is R¹SO₂—, R¹SO—, R¹S—, R¹CO—, R⁸—C(═O)—NH—, or R⁸—SO₂—NH—;
R¹ is alkyl, alkenyl, or alkynyl, each of which is optionally substituted by one or more aliphatic group substituents,
   cycloalkyl, cycloalkenyl, aryl, heteroaryl, heterocyclyl, heterocyclenyl, or multicyclic alkaryl, each of which is optionally substituted by one or more ring group substituents, or
   —NR′R″ when R is R¹SO₂— or R¹CO—;
   R′ is hydrogen,
      aryl, heteroaryl, cycloalkyl, cycloalkenyl, heterocyclyl, heterocyclenyl, or multicyclic alkaryl, each of which is optionally substituted by one or more ring group substituents, or
      alkyl, alkenyl or alkynyl, each of which is optionally substituted by one or more aliphatic group substituents;
   R″ is hydrogen, alkyl, alkenyl or alkynyl;
R² is hydrogen, halo, alkyl, alkenyl, alkynyl, haloalkyl, haloalkenyl, haloalkynyl, alkoxy, alkenyloxy or alkynyloxy;
R³ is acyl, cyano, carboxy, acid bioisostere, —C(O)—NY¹Y²,
   aroyl or heteroaroyl, each of which is optionally substituted by one or more ring group substituents,
   alkyl, alkenyl or alkynyl, each of which is optionally substituted by one or more aliphatic group substituents, or
   alkoxy, alkenyloxy or alkynyloxy, each of which is optionally substituted by one or more aliphatic group substituents;
   Y¹ and Y² are each independently hydrogen, alkylsulfonyl, arylsulfonyl, arylamino, heteroarylsulfonyl, heteroarylamino, or
      alkyl, alkenyl or alkynyl, each of which is optionally substituted by one or more aliphatic substituent groups;
R⁴ is hydrogen, acyl, aroyl, heteroaryl, alkylsulfonyl, arylsulfonyl, arylalkylsulfonyl, heteroarylsulfonyl, heteroarylalkylsulfonyl, —C(O)—NY⁴Y⁵, —C(O)—O—Y⁶,
   alkyl, alkenyl or alkynyl, each of which is optionally substituted by aryl, heteroaryl, carboxy, alkoxycarbonyl, aminocarbonyl, alkylaminocarbonyl, dialkylaminocarbonyl, aroyl, heteroaroyl or acyl, or
   (C₂-C₆)-alkyl, alkenyl or alkynyl, each of which is substituted by halo, hydroxy, alkoxy, amino, alkylamino or dialkylamino;
   Y⁴ and Y⁵ are each independently hydrogen, alkyl, alkenyl or alkynyl;
   Y⁶ is alkyl, alkenyl or alkynyl;
R⁵ is hydrogen, halo, carboxy, cyano, nitro, hydroxy, alkyl, alkenyl, alkynyl, haloalkyl, haloalkenyl, haloalkynyl, alkoxy, alkenyloxy, alkynyloxy, haloalkoxy, haloalkenyloxy or haloalkynyloxy;
R⁶ and R⁷ are each independently, hydrogen, alkyl, alkenyl or alkynyl;
R⁸ is alkyl, alkenyl, or alkynyl, each of which is optionally substituted by one or more aliphatic group substituents, or
   aryl, heteroaryl, cycloalkyl, cycloalkenyl, heterocyclyl, heterocyclenyl, or multicyclic alkaryl, each of which is optionally substituted by one or more ring group substituents; and
n is 1 to 6, or 0 when R³ is carboxy, acid bioisostere, or —C(O)—NY¹Y²;
   provided that when R¹ is amino, then R⁴ is hydrogen and n is 1 to 6;
      or a pharmaceutically acceptable salt, hydrate, or solvate thereof, a pharmaceutically acceptable prodrug thereof, or a pharmaceutically acceptable salt, hydrate or solvate of the prodrug.

Another aspect of the present invention is a pharmaceutical composition comprising, a pharmaceutically effective amount of one or more compounds according to Formula (XVI), or a pharmaceutically acceptable salt, hydrate, or solvate thereof, a pharmaceutically acceptable prodrug thereof, or a pharmaceutically acceptable salt, hydrate or solvate of the prodrug, in admixture with a pharmaceutically acceptable carrier.

Another aspect of the present invention is a method of treating a patient suffering from a PGD2-mediated disorder including, but not limited to, allergic disease (such as allergic rhinitis, allergic conjunctivitis, atopic dermatitis, bronchial asthma and food allergy), systemic mastocytosis, disorders accompanied by systemic mast cell activation, anaphylaxis shock, bronchoconstriction, bronchitis, urticaria, eczema, diseases accompanied by itch (such as atopic dermatitis and urticaria), diseases (such as cataract, retinal detachment, inflammation, infection and sleeping disorders) which are generated secondarily as a result of behavior accompanied by itch (such as scratching and beating), inflammation, chronic obstructive pulmonary diseases, ischemic reperfusion injury, cerebrovascular accident, chronic rheumatoid arthritis, pleurisy, ulcerative colitis and the like by administering to said patient a pharmaceutically effective amount of a compound according to Formula (XVI), or a pharmaceutically acceptable salt, hydrate, or solvate thereof, a pharmaceutically acceptable prodrug thereof, or a pharmaceutically acceptable salt, hydrate or solvate of the prodrug.

### DETAILED DESCRIPTION OF THE INVENTION

### Definition of the Terms

As used above, and throughout the description of the invention, the following terms, unless otherwise indicated, shall be understood to have the following meanings:

“Acid bioisostere” means a group which has chemical and physical similarities producing broadly similar biological properties to a carboxy group (see Lipinski, Annual Reports in Medicinal Chemistry, “Bioisosterism In Drug Design” 21, 283 (1986); Yun, Hwahak Sekye, “Application of Bioisosterism to New Drug Design” 33, 576-579, (1933); Zhao, Huaxue Tongbao, “Bioisosteric Replacement And Development Of Lead Compounds In Drug Design” 34-38, (1995); Graham, Theochem, “Theoretical Studies Applied To Drug Design ab initio Electronic Distributions In Bioisosteres” 343, 105-109, (1995)). Exemplary acid bioisosteres include —C(O)—NHOH, —C(O)—CH₂OH, —C(O)—CH₂SH, —C(O)—NH—CN, sulfo, phosphono, alkylsulfonylcarbamoyl, tetrazolyl, arylsulfonylcarbamoyl, N-methoxycarbamoyl, heteroarylsulfonylcarbamoyl, 3-hydroxy-3-cyclobutene-1,2-dione, 3,5-dioxo-1,2,4-oxadiazolidinyl, 5-oxo-4,5-dihydro-1,3,4-oxadiazol-2-yl, or hydroxyheteroaryl such as 3-hydroxyisoxazolyl, 3-hydroxy-1-methylpyrazolyl, and the like.

“Acyl” means H—CO— or (aliphatic or cyclyl)-CO—. Particular acyl includes lower alkanoyl that contains a lower alkyl. Exemplary acyl includes formyl, acetyl, propanoyl, 2-methylpropanoyl, butanoyl, palmitoyl, acryloyl, propynoyl, and cyclohexylcarbonyl.

“Aliphatic” means alkyl, alkenyl or alkynyl.

“Aliphatic group substituent(s)” include acyl, halo, nitro, cyano, hydroxy, alkoxy, alkenyloxy, alkynyloxy, haloalkoxy, haloalkenyloxy, haloalkynyloxy, aryloxy, heteroaryloxy, amino, alkylamino, dialkylamino, arylamino, heteroarylamino, carboxy, alkoxycarbonyl, aryloxycarbonyl, heteroaryloxycarbonyl, arylalkyloxycarbonyl, heteroarylalkyloxycarbonyl, aminocarbonyl, alkylaminocarbonyl, dialkylaminocarbonyl, aroyl, heteroaroyl, cycloalkyl, cycloalkenyl, aryl, heteroaryl, heterocyclyl, heterocyclenyl, or multicyclic alkaryl, wherein the aryloxy, heteroaryloxy, aryloxycarbonyl, heteroaryloxycarbonyl, arylalkyloxycarbonyl, heteroarylalkyloxycarbonyl, arylamino, heteroarylamino, aroyl, heteroaroyl, cycloalkyl, cycloalkenyl, aryl, heteroaryl, heterocyclyl, heterocyclenyl, or multicyclic alkaryl is independently optionally substituted by one or more ring group substituents.

“Alkenyl” means a straight or branched aliphatic hydrocarbon group containing a carbon-carbon double bond and having 2 to about 15 carbon atoms. Particular alkenyl has 2 to about 12 carbon atoms. More particular alkenyl has 2 to about 4 carbon atoms. Branched means that one or more lower alkyl groups such as methyl, ethyl or propyl are attached to a linear alkenyl chain. “Lower alkenyl” means about 2 to about 4 carbon atoms in the chain that may be straight or branched. Exemplary alkenyl includes ethenyl, propenyl, n-butenyl, i-butenyl, 3-methylbut-2-enyl, n-pentenyl, heptenyl, octenyl, cyclohexylbutenyl, and decenyl.

“Alkenyloxy” means an alkenyl-O— group wherein the alkenyl group is as herein described. Exemplary alkenyloxy groups include allyloxy, 3-butenyloxy, and the like.

“Alkoxy” means allyl-O—. Exemplary alkoxy includes methoxy, ethoxy, n-propoxy, i-propoxy, n-butoxy, and heptoxy.

“Alkoxycarbonyl” means alkyl-O—CO—. Exemplary alkoxycarbonyl includes methoxycarbonyl, ethoxycarbonyl, and t-butyloxycarbonyl.

“Alkyl” means straight or branched aliphatic hydrocarbon having 1 to about 20 carbon atoms. Particular alkyl has 1 to about 12 carbon atoms. More particular alkyl is lower alkyl. Branched means that one or more lower alkyl groups such as methyl, ethyl or propyl are attached to a linear alkyl chain.

“Lower alkyl” means 1 to about 4 carbon atoms in a linear alkyl chain that may be straight or branched.

“Alkylamino” means alkyl-NH—. Particular alkylamino is (C₁-C₆)-alkylamino. Exemplary alkylamino includes methylamino and ethylamino.

“Alkylene” means a straight or branched bivalent hydrocarbon having from 1 to about 15 carbon atoms. Particular alkylene is the lower alkylene having from 1 to about 6 carbon atoms. Exemplary alkenylene includes methylene, ethylene, propylene, and butylenes.

“Alkylsulfonyl” means alkyl-SO₂—. Particular alkylsulfonyl is (C₁-C₆)-alkylsulfonyl. Exemplary alkylsulfonyl includes CH₃—SO₂—, and CH₃CH₂—SO₂—.

“Alkylthio” means an alkyl-S—. Exemplary alkylthio includes CH₃—S—.

“Alkynyl” means straight or branched aliphatic hydrocarbon containing a carbon-carbon triple bond and having 2 to about 15 carbon atoms. Particular alkynyl has 2 to about 12 carbon atoms. More particular alkynyl has 2 to about 6 carbon atoms. Branched means that one or more lower alkyl such as methyl, ethyl or propyl are attached to a linear alkynyl chain. “Lower alkynyl” means 2 to about 4 carbon atoms in a linear alkynyl chain that may be straight or branched. Exemplary alkynyl includes ethynyl, propynyl, n-butynyl, 2-butynyl, 3-methylbutynyl, n-pentynyl, heptynyl, octynyl, and decynyl.

“Alkynyloxy” means an alkynyl-O— group wherein the alkenyl group is as herein described. Exemplary alkynyloxy groups include 2-propynyloxy, 3-butynyloxy, and the like.

“Aroyl” means aryl-CO—. Exemplary aroyl includes benzoyl, and 1- and 2-naphthoyl.

“Aryl” means an aromatic monocyclic or multicyclic ring system of about 6 to about 14 carbon atoms. Particular aryl include about 6 to about 10 carbon atoms. Exemplary aryl include phenyl and naphthyl.

“Arylalkoxy” means arylalkyl-O—. Exemplary arylalkoxy includes benzyloxy and 1- or 2-naphthylenemethoxy.

“Arylalkoxycarbonyl” means arylalkyl-O—CO—. Exemplary arylalkoxycarbonyl includes phenoxycarbonyl and naphthoxycarbonyl.

“Arylalkyl” means aryl-alkyl—. Particular arylalkyl contains a (C₁-C₆)-alkyl moiety. Exemplary arylalkyl includes benzyl, 2-phenethyl and naphthylenemethyl.

“Arylalkylsulfonyl” means aryl-alkyl-SO₂—. Particular arylalkylsulfonyl contains a (C₁-C₆)-alkyl moiety. Exemplary arylalkylsulfonyl includes benzylsulfonyl.

“Arylalkylthio” means arylalkyl-S—. Exemplary arylalkylthio includes benzylthio.

“Arylamino” means aryl-NH—. Exemplary arylamino includes phenylamino.

“Arylcycloalkenyl” means a fused aryl and cycloalkenyl. Particular arylcycloalkenyl is one wherein the aryl thereof is phenyl and the cycloalkenyl consists of about 5 to about 7 ring atoms. An arylcycloalkenyl is bonded through any atom of the cycloalkenyl moiety thereof capable of such bonding. Exemplary arylcycloalkenyl includes 1,2-dihydronaphthylene and indene.

“Arylcycloalkyl” means a fused aryl and cycloalkyl. Particular arylcycloalkyl is one wherein the aryl thereof is phenyl and the cycloalkyl consists of about 5 to about 6 ring atoms. An arylcycloalkyl is bonded through any atom of the cycloalkyl moiety thereof capable of such bonding. Exemplary arylcycloalkyl includes 1,2,3,4-tetrahydro-naphthylene.

“Arylheterocyclenyl” means a fused aryl and heterocyclenyl. Particular arylheterocyclenyl is one wherein the aryl thereof is phenyl and the heterocyclenyl consists of about 5 to about 6 ring atoms. An arylheterocyclenyl is bonded through any atom of the heterocyclenyl thereof capable of such bonding. The designation of the aza, oxa or thio as a prefix before the heterocyclenyl portion of the arylheterocyclenyl defines that at least a nitrogen, oxygen or sulfur atom is present, respectively, as a ring atom. The nitrogen atom of an arylheterocyclenyl may be a basic nitrogen atom. The nitrogen or sulfur atom of the heterocyclenyl portion of the arylheterocyclenyl may also be optionally oxidized to the corresponding N-oxide, S-oxide or S,S-dioxide. Exemplary arylheterocyclenyl includes 3H-indolinyl, 1H-2-oxoquinolyl, 2H-1-oxoisoquinolyl, 1,2-di-hydroquinolinyl, 3,4-dihydroquinolinyl, 1,2-dihydroisoquinolinyl, and 3,4-dihydroisoquinolinyl.

“Arylheterocyclyl” means a fused aryl and heterocyclyl. Particular heterocyclylaryl is one wherein the aryl thereof is phenyl and the heterocyclyl consists of about 5 to about 6 ring atoms. An arylheterocyclyl is bonded through any atom of the heterocyclyl moiety thereof capable of such bonding. The designation of the aza, oxa or thio as a prefix before heterocyclyl portion of the arylheterocyclyl defines that at least a nitrogen, oxygen or sulfur atom is present, respectively, as a ring atom. The nitrogen atom of an arylheterocyclyl may be a basic nitrogen atom. The nitrogen or sulfur atom of the heterocyclyl portion of the arylheterocyclyl may also be optionally oxidized to the corresponding N-oxide, S-oxide or S,S-dioxide. Exemplary arylheterocyclyl includes indolinyl, 1,2,3,4-tetrahydroisoquinoline, 1,2,3,4-tetrahydroquinoline, 1H-2,3-dihydroisoindol-2-yl, 2,3-dihydrobenz[f]isoindol-2-yl, and 1,2,3,4-tetrahydrobenz[g]-isoquinolin-2-yl.

“Aryloxy” means an aryl-O—. Exemplary aryloxy includes phenoxy and naphthoxy.

“Aryloxycarbonyl” means aryl-O—CO—. Exemplary aryloxycarbonyl includes phenoxycarbonyl and naphthoxycarbonyl.

“Arylsulfonyl” means aryl-SO₂—. Exemplary arylsulfonyl includes phenylsulfonyl and naphthylsulfonyl.

“Arylthio” means aryl-S—. Exemplary arylthio includes phenylthio and naphthylthio.

“Compounds of the present invention”, and equivalent expressions, are meant to embrace compounds of Formula (XVI) as hereinbefore described, which expression includes the prodrugs, the pharmaceutically acceptable salts, and the solvates, e.g., hydrates, where the context so permits. Similarly, reference to intermediates, whether or not they themselves are claimed, is meant to embrace their salts, and solvates, where the context so permits.

“Cycloalkenyl” means a non-aromatic mono- or multicyclic ring system of about 3 to about 10 carbon atoms, preferably of about 5 to about 10 carbon atoms, and which contains at least one carbon-carbon double bond. Particular rings of the ring system include about 5 to about 6 ring atoms; and such particular ring sizes are also referred to as “lower”. Exemplary monocyclic cycloalkenyl includes cyclopentenyl, cyclohexenyl, and cycloheptenyl. An exemplary multicyclic cycloalkenyl is norbornylenyl.

“Cycloalkenylaryl” means a fused aryl and cycloalkenyl. Particular cycloalkenylaryl is one wherein the aryl thereof is phenyl and the cycloalkenyl consists of about 5 to about 6 ring atoms. A cycloalkenylaryl is bonded through any atom of the aryl moiety thereof capable of such bonding. Exemplary cycloalkenylaryl includes 1,2-dihydronaphthylene and indene.

“Cycloalkenylheteroaryl” means a fused heteroaryl and cycloalkenyl. Particular cycloalkenylheteroaryl is one wherein the heteroaryl thereof consists of about 5 to about 6 ring atoms and the cycloalkenyl consists of about 5 to about 6 ring atoms. A cycloalkenylheteroaryl is bonded through any atom of the heteroaryl thereof capable of such bonding. The designation of the aza, oxa or thio as a prefix before heteroaryl portion of the cycloalkenylheteroaryl defines that at least a nitrogen, oxygen or sulfur atom is present, respectively, as a ring atom. The nitrogen atom of a cycloalkenylheteroaryl may be a basic nitrogen atom. The nitrogen atom of the heteroaryl portion of the cycloalkenylheteroaryl may also be optionally oxidized to the corresponding N-oxide. Exemplary cycloalkenylheteroaryl includes 5,6-dihydroquinolyl, 5,6-dihydroisoquinolyl, 5,6-dihydroquinoxalinyl, 5,6-dihydroquinazolinyl, 4,5-dihydro-1H-benzimidazolyl, and 4,5-di-hydrobenzoxazolyl.

“Cycloalkyl” means a non-aromatic mono- or multicyclic saturated ring system of about 3 to about 10 carbon atoms, preferably of about 5 to about 10 carbon atoms. Particular ring systems include about 5 to about 7 ring atoms; and such particular ring systems are also referred to as “lower”. Exemplary monocyclic cycloalkyl includes cyclopentyl, cyclohexyl, and cycloheptyl. Exemplary multicyclic cycloalkyl includes 1-decalin, norbornyl, and adamant-(1- or 2-)yl.

“Cycloalkylaryl” means a fused aryl and cycloalkyl. Particular cycloalkylaryl is one wherein the aryl thereof is phenyl and the cycloalkyl consists of about 5 to about 6 ring atoms. A cycloalkylaryl is bonded through any atom of the cycloalkyl moiety thereof capable of such bonding. Exemplary cycloalkylaryl includes 1,2,3,4-tetrahydro-naphthylene.

“Cycloalkylene” means a bivalent cycloalkyl group having about 4 to about 8 carbon atoms. Particular cycloalkylene includes about 5 to about 7 ring atoms; and such particular ring systems are also referred to as “lower”. The points of binding on the cycloalkylene group include 1,1-, 1,2-, 1,3-, or 1,4-binding patterns, and where applicable the stereochemical relationship of the points of binding is either cis or trans. Exemplary monocyclic cycloalkylene includes (1,1-, 1,2-, or 1,3-)cyclohexylene and (1,1- or 1,2-)cyclopentylene.

“Cycloalkylheteroaryl” means a fused heteroaryl and cycloalkyl. Particular cycloalkylheteroaryl is one wherein the heteroaryl thereof consists of about 5 to about 6 ring atoms and the cycloalkyl consists of about 5 to about 6 ring atoms. A cycloalkylheteroaryl is bonded through any atom of the heteroaryl thereof capable of such bonding. The designation of the aza, oxa or thio as a prefix before heteroaryl portion of the fused cycloalkylheteroaryl defines that at least a nitrogen, oxygen or sulfur atom is present, respectively, as a ring atom. The nitrogen atom of a cycloalkylheteroaryl may be a basic nitrogen atom. The nitrogen atom of the heteroaryl portion of the cycloalkylheteroaryl may also be optionally oxidized to the corresponding N-oxide. Exemplary cycloalkylheteroaryl includes 5,6,7,8-tetrahydroquinolinyl, 5,6,7,8-tetra-hydroisoquinolyl, 5,6,7,8-tetrahydroquinoxalinyl, 5,6,7,8-tetrahydroquinazolyl, 4,5,6,7-tetrahydro-1H-benzimidazolyl, and 4,5,6,7-tetrahydrobenzoxazolyl.

“Cyclyl” means cycloalkyl, cycloalkenyl, heterocyclyl or heterocyclenyl.

“Dialkylamino” means (alkyl)₂-N—. Particular dialkylamino is (C₁-C₆alkyl)₂-N—. Exemplary dialkylamino groups include dimethylamino, diethylamino and methylethylamino.

“Halo” or “halogen” means fluoro, chloro, bromo, or iodo. Particular halo or halogen are fluoro or chloro.

“Haloalkoxy” means alkoxy substituted by one to three halo groups. Particular haloalkoxy are loweralkoxy substituted by one to three halogens. Most particular haloalkoxy are loweralkoxy substituted by one halogen.

“Haloalkenyloxy” means alkenyloxy substituted by one to three halo groups. Particular haloalkenyloxy are loweralkenyloxy substituted by one to three halogens. Most particular haloalkoxy are loweralkenyloxy substituted by one halogen.

“Haloalkynyloxy” means alkynyloxy substituted by one to three halo groups. Particular haloalkynyloxy are loweralkynyloxy substituted by one to three halogens. Most particular haloalkynyloxy are loweralkynyloxy substituted by one halogen.

“Haloalkenyl” means alkenyl substituted by one to three halo groups. Particular haloalkenyl are loweralkenyl substituted by one to three halogens. Most particular haloalkyl are loweralkyl substituted by one halogen.

“Haloalkyl” means alkyl substituted by one to three halo groups. Particular haloalkyl are loweralkyl substituted by one to three halogens. Most particular haloalkyl are loweralkyl substituted by one halogen.

“Haloalkynyl” means alkynyl substituted by one to three halo groups. Particular haloalkynyl are loweralkynyl substituted by one to three halogens. Most particular haloalkynyl are loweralkynyl substituted by one halogen.

“Heteroaroyl” means heteroaryl-CO—. Exemplary heteroaroyl includes thiophenoyl, nicotinoyl, pyrrol-2-ylcarbonyl, and pyridinoyl.

“Heteroaryl” means an aromatic monocyclic or multicyclic ring system of about 5 to about 14 carbon atoms, in which one or more of the carbon atoms in the ring system is/are hetero element(s) other than carbon, for example nitrogen, oxygen or sulfur. Preferably aromatic ring systems include about 5 to about 10 carbon atoms, and include 1 to 3 heteroatoms. Most preferred ring sizes of rings of the ring system include about 5 to about 6 ring atoms. The designation of the aza, oxa or thio as a prefix before heteroaryl defines that at least a nitrogen, oxygen or sulfur atom is present, respectively, as a ring atom. A nitrogen atom of a heteroaryl may be a basic nitrogen atom and may also be optionally oxidized to the corresponding N-oxide. When a heteroaryl is substituted by a hydroxy group, it also includes its corresponding tautomer. Exemplary heteroaryl includes pyrazinyl, thienyl, isothiazolyl, oxazolyl, pyrazolyl, furanyl, pyrrolyl, 1,2,4-thiadiazolyl, pyridazinyl, quinoxalinyl, phthalazinyl, imidazo[1,2-a]pyridine, imidazo[2,1-b]thiazolyl, benzofuranyl, azaindolyl, benzimidazolyl, benzothienyl, thienopyridyl, thienopyrimidyl, pyrrolopyridyl, imidazopyridyl, benzoazaindolyl, 1,2,4-triazinyl, benzothiazolyl, imidazolyl, indolyl, indolizinyl, isoxazolyl, isoquinolinyl, isothiazolyl, oxadiazolyl, pyrazinyl, pyridazinyl, pyrazolyl, pyridyl, pyrimidinyl, pyrrolyl, quinazolinyl, quinolinyl, 1,3,4-thiadiazolyl, thiazolyl, thienyl, and triazolyl.

“Heteroarylalkyl” means heteroaryl-alkyl-. Particular heteroarylalkyl contains a (C₁-C₄)-alkyl moiety. Exemplary heteroarylalkyl includes tetrazol-5-ylmethyl.

“Heteroarylalkoxy” means heteroaryl-alkyl-O—.

“Heteroarylalkoxycarbonyl” means heteroarylalkyl-O—CO—.

“Heteroarylalkylsulfonyl” means heteroaryl-alkyl-SO₂—. Particular heteroarylalkylsulfonyl contains a (C₁-C₆)-alkyl moiety.

“Heteroarylalkylthio” means heteroarylalkyl-S—. Particular heteroarylalkylthio contains a (C₁-C₆)-alkyl moiety.

“Heteroarylamino” means heteroaryl-NH—.

“Heteroarylcycloalkenyl” means a fused heteroaryl and cycloalkenyl. Particular heteroarylcycloalkenyl is one wherein the heteroaryl thereof consists of about 5 to about 6 ring atoms and the cycloalkenyl consists of about 5 to about 6 ring atoms. A heteroarylcycloalkenyl is bonded through any atom of the cycloalkenyl thereof capable of such bonding. The designation of the aza, oxa or thio as a prefix before heteroaryl portion of the heteroarylcycloalkenyl defines that at least a nitrogen, oxygen or sulfur atom is present, respectively, as a ring atom. The nitrogen atom of a heteroarylcycloalkenyl may be a basic nitrogen atom. The nitrogen atom of the heteroaryl portion of the heteroarylcycloalkenyl may also be optionally oxidized to the corresponding N-oxide. Exemplary heteroarylcycloalkenyl includes 5,6-dihydroquinolyl, 5,6-dihydroisoquinolyl, 5,6-dihydroquinoxalinyl, 5,6-dihydroquinazolinyl, 4,5-dihydro-1H-benzimidazolyl, and 4,5-di-hydrobenzoxazolyl.

“Heteroarylcycloalkyl” means a fused heteroaryl and cycloalkyl. Particular heteroarylcycloalkyl is one wherein the heteroaryl thereof consists of about 5 to about 6 ring atoms and the cycloalkyl consists of about 5 to about 6 ring atoms. A heteroarylcycloalkyl is bonded through any atom of the cycloalkyl thereof capable of such bonding. The designation of the aza, oxa or thio as a prefix before heteroaryl portion of the fused heteroarylcycloalkyl defines that at least a nitrogen, oxygen or sulfur atom is present, respectively, as a ring atom. The nitrogen atom of a heteroarylcycloalkyl may be a basic nitrogen atom. The nitrogen atom of the heteroaryl portion of the heteroarylcycloalkyl may also be optionally oxidized to the corresponding N-oxide. Exemplary heteroarylcycloalkyl includes 5,6,7,8-tetrahydroquinolinyl, 5,6,7,8-tetra-hydroisoquinolyl, 5,6,7,8-tetrahydroquinoxalinyl, 5,6,7,8-tetrahydroquinazolyl, 4,5,6,7-tetrahydro-1H-benzimidazolyl, and 4,5,6,7-tetrahydrobenzoxazolyl

“Heteroarylheterocyclenyl” means a fused heteroaryl and heterocyclenyl. Particular heteroarylheterocyclenyl is one wherein the heteroaryl thereof consists of about 5 to about 6 ring atoms and the heterocyclenyl consists of about 5 to about 6 ring atoms. A heteroarylheterocyclenyl is bonded through any atom of the heterocyclenyl thereof capable of such bonding. The designation of the aza, oxa or thio as a prefix before the heteroaryl or heterocyclenyl portion of the heteroarylheterocyclenyl defines that at least a nitrogen, oxygen or sulfur atom is present, respectively, as a ring atom. The nitrogen atom of a heteroarylazaheterocyclenyl may be a basic nitrogen atom. The nitrogen or sulfur atom of the heteroaryl portion of the heteroarylheterocyclyl may also be optionally oxidized to the corresponding N-oxide. The nitrogen or sulfur atom of the heteroaryl or heterocyclyl portion of the heteroarylheterocyclyl may also be optionally oxidized to the corresponding N-oxide, S— oxide or S,S-dioxide. Exemplary heteroarylheterocyclenyl includes 7,8-dihydro[1,7]naphthyridinyl, 1,2-dihydro[2,7]-naphthyridinyl, 6,7-dihydro-3H-imidazo[4,5-c]pyridyl, 1,2-dihydro-1,5-naphthyridinyl, 1,2-dihydro-1,6-naphthyridinyl, 1,2-dihydro-1,7-naphthyridinyl, 1,2-dihydro-1,8-naphthyridinyl, and 1,2-dihydro-2,6-naphthyridinyl.

“Heteroarylheterocyclyl” means a fused heteroaryl and heterocyclyl. Particular heteroarylheterocyclyl is one wherein the heteroaryl thereof consists of about 5 to about 6 ring atoms and the heterocyclyl consists of about 5 to about 6 ring atoms. A heteroarylheterocyclyl is bonded through any atom of the heterocyclyl thereof capable of such bonding. The designation of the aza, oxa or thio as a prefix before the heteroaryl or heterocyclyl portion of the fused heteroarylheterocyclyl defines that at least a nitrogen, oxygen or sulfur atom is present, respectively, as a ring atom. The nitrogen atom of a fused heteroarylheterocyclyl may be a basic nitrogen atom. The nitrogen or sulfur atom of the heteroaryl portion of the heteroarylheterocyclyl may also be optionally oxidized to the corresponding N-oxide. The nitrogen or sulfur atom of the heteroaryl or heterocyclyl portion of the heteroarylheterocyclyl may also be optionally oxidized to the corresponding N-oxide, S-oxide or S,S-dioxide. Exemplary heteroarylheterocyclyl includes 2,3-dihydro-1H-pyrrol[3,4-b]quinolin-2-yl, 1,2,3,4-tetrahydrobenz[b][1,7]naphthyridin-2-yl, 1,2,3,4-tetrahydrobenz[b][1,6]naphthyridin-2-yl, 1,2,3,4-tetra-hydro-9H-pyrido[3,4-b]indol-2-yl, 1,2,3,4-tetrahydro-9H-pyrido[4,3-b]indol-2-yl, 2,3-dihydro-1H-pyrrolo[3,4-b ]indol-2-yl, 1H-2,3,4,5-tetrahydroazepino[3,4-b]indol-2-yl, 1H-2,3,4,5-tetra-hydroazepino[4,3-b]indol-3-yl, 1H-2,3,4,5-tetrahydroazepino[4,5-b]indol-2 yl, 5,6,7,8-tetra-hydro[1,7]naphthyridyl, 1,2,3,4-tetrhydro[2,7]naphthyridyl, 2,3-dihydro[1,4]dioxino[2,3-b]pyridyl, 2,3-dihydro-[1,4]dioxino[2,3-b]pyridyl, 3,4-dihydro-2H-1-oxa[4,6]diazanaphthalenyl, 4,5,6,7-tetrahydro-3H-imidazo[4,5-c]pyridyl, 6,7-dihydro[5,8]diazanaphthalenyl, 1,2,3,4-tetrahydro[1,5]-naphthyridinyl, 1,2,3,4-tetrahydro[1,6]naphthyridinyl, 1,2,3,4-tetrahydro[1,7]naphthyridinyl, 1,2,3,4-tetrahydro[1,8]naphthyridinyl, and 1,2,3,4-tetra-hydro[2,6]naphthyridinyl.

“Heteroaryloxy” means heteroaryl-O—. Exemplary heteroaryloxy includes pyridyloxy.

“Heterocyclenyl” means a non-aromatic monocyclic or multicyclic hydrocarbon ring system of about 3 to about 10 carbon atoms, in which one or more of the carbon atoms in the ring system is/are hetero element(s) other than carbon, for example nitrogen, oxygen or sulfur atoms, and which contains at least one carbon-carbon double bond or carbon-nitrogen double bond. Preferably, the non-aromatic ring system includes about 5 to about 10 carbon atoms, and 1 to 3 heteroatoms. Most preferred ring sizes of rings of the ring system include about 5 to about 6 ring atoms; and such particular ring sizes are also referred to as “lower”. The designation of the aza, oxa or thio as a prefix before heterocyclenyl defines that at least a nitrogen, oxygen or sulfur atom is present, respectively, as a ring atom. The nitrogen atom of a heterocyclenyl may be a basic nitrogen atom. The nitrogen or sulfur atom of the heterocyclenyl may also be optionally oxidized to the corresponding N-oxide, S-oxide or S,S-dioxide. Exemplary monocyclic azaheterocyclenyl includes 1,2,3,4-tetrahydrohydropyridine, 1,2-dihydropyridyl, 1,4-dihydropyridyl, 1,2,3,6-tetra-hydropyridine, 1,4,5,6-tetrahydro-pyrimidine, 2-pyrrolinyl, 3-pyrrolinyl, 2-imidazolinyl, and 2-pyrazolinyl. Exemplary oxaheterocyclenyl includes 3,4-dihydro-2H-pyran, dihydrofuranyl, and fluorodihydro-furanyl. An exemplary multicyclic oxaheterocyclenyl is 7-oxabicyclo[2.2.1]heptenyl. Exemplary monocyclic thioheterocyclenyl includes dihydrothiophenyl and dihydrothiopyranyl.

“Heterocyclenylaryl” means a fused aryl and heterocyclenyl. Particular heterocyclenylaryl is one wherein the aryl thereof is phenyl and the heterocyclenyl consists of about 5 to about 6 ring atoms. A heterocyclenylaryl is bonded through any atom of the aryl thereof capable of such bonding. The designation of the aza, oxa or thio as a prefix before heterocyclenyl portion of the fused heterocyclenylaryl defines that at least a nitrogen, oxygen or sulfur atom is present, respectively, as a ring atom. The nitrogen atom of a heterocyclenylaryl may be a basic nitrogen atom. The nitrogen or sulfur atom of the heterocyclenyl portion of the heterocyclenylaryl may also be optionally oxidized to the corresponding N-oxide, S-oxide or S,S-dioxide. Exemplary heterocyclenylaryl include 3H-indolinyl, IH-2-oxoquinolyl, 2H-1-oxoisoquinolyl, 1,2-di-hydroquinolinyl, 3,4-dihydroquinolinyl, 1,2-dihydroisoquinolinyl, and 3,4-dihydroisoquinolinyl.

“Heterocyclenylheteroaryl” means a fused heteroaryl and heterocyclenyl. Particular heterocyclenylheteroaryl is one wherein the heteroaryl thereof consists of about 5 to about 6 ring atoms and the heterocyclenyl consists of about 5 to about 6 ring atoms. A heterocyclenylheteroaryl is bonded through any atom of the heteroaryl thereof capable of such bonding. The designation of the aza, oxa or thio as a prefix before the heteroaryl or heterocyclenyl portion of the heterocyclenylheteroaryl define that at least a nitrogen, oxygen or sulfur atom is present, respectively, as a ring atom. The nitrogen atom of an azaheterocyclenylheteroaryl may be a basic nitrogen atom. The nitrogen or sulfur atom of the heteroaryl portion of the heterocyclenylheteroaryl may also be optionally oxidized to the corresponding N-oxide. The nitrogen or sulfur atom of the heteroaryl or heterocyclyl portion of the heterocyclenylheteroaryl may also be optionally oxidized to the corresponding N-oxide, S-oxide or S,S-dioxide. Exemplary heterocyclenylheteroaryl includes 7,8-dihydro[1,7]naphthyridinyl, 1,2-dihydro[2,7]-naphthyridinyl, 6,7-dihydro-3H-imidazo[4,5-c]pyridyl, 1,2-dihydro-1,5-naphthyridinyl, 1,2-dihydro-1,6-naphthyridinyl, 1,2-dihydro-1,7-naphthyridinyl, 1,2-dihydro-1,8-naphthyridinyl and 1,2-dihydro-2,6-naphthyridinyl.

“Heterocyclyl” means a non-aromatic saturated monocyclic or multicyclic ring system of about 3 to about 10 carbon atoms, in which one or more of the atoms in the ring system is/are hetero element(s) other than carbon, for example nitrogen, oxygen or sulfur. Preferably, the ring system contains about to about 10 carbon atoms, and from 1 to 3 heteroatoms. Particular ring sizes of rings of the ring system include about 5 to about 6 ring atoms; and such particular ring sizes are also referred to as “lower”. The designation of the aza, oxa or thio as a prefix before heterocyclyl define that at least a nitrogen, oxygen or sulfur atom is present respectively as a ring atom. The nitrogen atom of a heterocyclyl may be a basic nitrogen atom. The nitrogen or sulfur atom of the heterocyclyl may also be optionally oxidized to 20 the corresponding N-oxide, S-oxide or S,S-dioxide. Exemplary monocyclic heterocyclyl includes piperidyl, pyrrolidinyl, piperazinyl, morpholinyl, thiomorpholinyl, thiazolidinyl, 1,3-dioxolanyl, 1,4-dioxanyl, THFyl, tetrahydrothiophenyl, and tetrahydrothiopyranyl.

“Heterocyclylaryl” means a fused aryl and heterocyclyl. Particular heterocyclylaryl is one wherein the aryl thereof is phenyl and the heterocyclyl consists of about 5 to about 6 ring atoms. A heterocyclylaryl is bonded through any atom of the aryl moiety thereof capable of such bonding. The designation of the aza, oxa or thio as a prefix before heterocyclyl portion of the heterocyclylaryl defines that at least a nitrogen, oxygen or sulfur atom is present, respectively, as a ring atom. The nitrogen atom of a heterocyclylaryl may be a basic nitrogen atom. The nitrogen or sulfur atom of the heterocyclyl portion of the heterocyclylaryl may also be optionally oxidized to the corresponding N-oxide, S-oxide or S,S-dioxide. Exemplary heterocyclylaryl includes indolinyl, 1,2,3,4-tetrahydroisoquinoline, 1,2,3,4-tetrahydroquinoline, 1H-2,3-dihydroisoindol-2-yl, and 2,3-dihydrobenz[f]isoindol-2-yl, and 1,2,3,4-tetrahydrobenz[g]-isoquinolin-2-yl.

“Heterocyclylheteroaryl” means a fused heteroaryl and heterocyclyl. Particular heterocyclylheteroaryl is one wherein the heteoraryl thereof consists of about 5 to about 6 ring atoms and the heterocyclyl consists of about 5 to about 6 ring atoms. A heterocyclylheteroaryl is bonded through any atom of the heterocyclyl thereof capable of such bonding. The designation of the aza, oxa or thio as a prefix before the heteroaryl or heterocyclyl portion of the heterocyclylheteroaryl defines that at least a nitrogen, oxygen or sulfur atom is present, respectively, as a ring atom. The nitrogen atom of a heterocyclylheteroaryl may be a basic nitrogen atom. The nitrogen or sulfur atom of the heteroaryl portion of the heterocyclylheteroaryl may also be optionally oxidized to the corresponding N-oxide. The nitrogen or sulfur atom of the heteroaryl or heterocyclyl portion of the heterocyclylheteroaryl may also be optionally oxidized to the corresponding N-oxide, S-oxide or S,S-dioxide. Exemplary heterocyclylheteroaryl includes 2,3-dihydro-1H-pyrrol[3,4-b]quinolin-2-yl, 1,2,3,4-tetrahydrobenz[b][1,7]naphthyridin-2-yl, 1,2,3,4-tetrahydrobenz[b][1,6]naphthyridin-2-yl, 1,2,3,4-tetra-hydro-9H-pyrido[3,4-b]indol-2-yl, 1,2,3,4-tetrahydro-9H-pyrido[4,3-b]indol-2-yl, 2,3-dihydro-1H-pyrrolo[3,4-b ]indol-2-yl, 1H-2,3,4,5-tetrahydroazepino[3,4-b]indol-2-yl, 1H-2,3,4,5-tetra-hydroazepino[4,3-b]indol-3-yl, 1H-2,3,4,5-tetrahydroazepino[4,5-b]indol-2-yl, 5,6,7,8-tetra-hydro[1,7]naphthyridyl, 1,2,3,4-tetrhydro[2,7]naphthyridyl, 2,3-dihydro[1,4]dioxino[2,3-b]pyridyl, 2,3-dihydro-[1,4]dioxino[2,3-b]pyridyl, 3,4-dihydro-2H-1-oxa[4,6]diazanaphthalenyl, 4,5,6,7-tetrahydro-3H-imidazo[4,5-c]pyridyl, 6,7-dihydro[5,8]diazanaphthalenyl, 1,2,3,4-tetrahydro[1,5]-naphthyridinyl, 1,2,3,4-tetrahydro[1,6]naphthyridinyl, 1,2,3,4-tetrahydro[1,7]naphthyridinyl, 1,2,3,4-tetrahydro[1,8]naphthyridinyl, and 1,2,3,4-tetra-hydro[2,6]naphthyridinyl.

“Multicyclic alkaryl” means a multicyclic ring system including at least one aromatic ring fused to at least one non-aromatic ring that may be saturated or unsaturated, and may also contain in the ring system one or more heteroatoms, such as nitrogen, oxygen or sulfur. Exemplary multicyclic alkaryl includes arylcycloalkenyl, arylcycloalkyl, arylheterocyclenyl, arylheterocyclyl, cycloalkenylaryl, cycloalkylaryl, cycloalkenylheteroaryl, cycloalkylheteroaryl, heteroarylcycloalkenyl, heteroarylcycloalkyl, heteroarylheterocyclenyl, heteroarylheterocyclyl, heterocyclenylaryl, heterocyclenylheteroaryl, heterocyclylaryl, and heterocyclylheteroaryl. Particular multicyclic alkaryl groups are bicyclic rings that include one aromatic ring fused to one non-aromatic ring and that also may contain in the ring system one or more heteroatoms, such as nitrogen, oxygen or sulfur.

“Patient” includes human and other mammals.

“Pharmaceutically acceptable prodrugs” as used herein refers to those prodrugs of the compounds of the present invention which are, within the scope of sound medical judgment, suitable for use in contact with the tissues of patients with undue toxicity, irritation, allergic response commensurate with a reasonable benefit/risk ratio, and effective for their intended use of the compounds of the invention. The term “prodrug” means a compound that is transformed in vivo to yield a compound of Formula (XVI) or a pharmaceutically acceptable salt, hydrate or solvate of the compound. The transformation may occur by various mechanisms, such as through hydrolysis in blood. The compounds bearing metabolically cleavable groups have the advantage that they may exhibit improved bioavailability as a result of enhanced solubility and/or rate of absorption conferred upon the parent compound by virtue of the presence of the metabolically cleavable group, thus, such compounds act as pro-drugs. A thorough discussion is provided in Design of Prodrugs, H. Bundgaard, ed., Elsevier (1985); Methods in Enzymology; K. Widder et al, Ed., Academic Press, 42, 309-396 (1985); A Textbook of Drug Design and Development, Krogsgaard-Larsen and H. Bandaged, ed., Chapter 5; “Design and Applications of Prodrugs” 113-191 (1991); Advanced Drug Delivery Reviews, H. Bundgard, 8, 1-38, (1992); J. Pharm. Sci., 77, 285 (1988); Chem. Pharm. Bull., N. Nakeya et al, 32, 692 (1984); Pro-drugs as Novel Delivery Systems, T. Higuchi and V. Stella, 14 A.C.S. Symposium Series, and Bioreversible Carriers in Drug Design, E. B. Roche, ed., American Pharmaceutical Association and Pergamon Press, 1987; J. Med. Chem., Vol. 47, No. 10, 1-12 (2004), which are incorporated herein by reference.

An example of the prodrugs of a compound of the present invention is an ester prodrug. “Ester prodrug” means a compound that is convertible in vivo by metabolic means (e.g., by hydrolysis) to a compound of Formula (XVI). For example, an ester prodrug of a compound of Formula (XVI) containing a carboxy group may be convertible by hydrolysis in vivo to the corresponding compound of Formula (XVI), such as methyl ester prodrug, ethyl ester prodrug or 2-dimethylamino-ethyl ester prodrug. Exemplary ester prodrugs are: 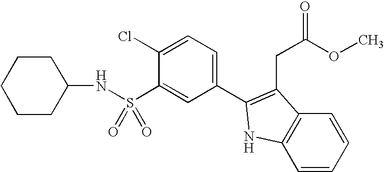
[2-(4-Chloro-3-cyclohexylsulfamoyl-phenyl)-1H-indol-3-yl]-acetic acid methyl ester; 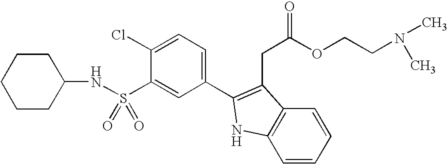
[2-(4-Chloro-3-cyclohexylsulfamoyl-phenyl)-1H-indol-3-yl]-acetic acid 2-dimethylamino-ethyl ester; 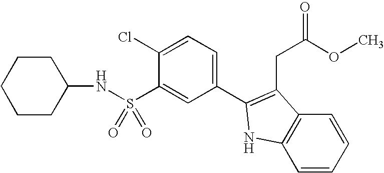
[2-(4-Chloro-3-cyclohexylsulfamoyl-phenyl)-1H-indol-3-yl]-acetic acid methyl ester; and 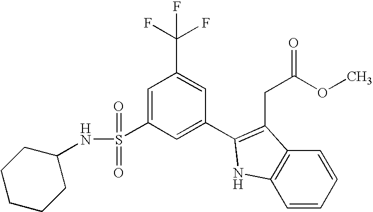
[2-(3-cyclohexylsulfamoyl-5-trifluoromethyl-phenyl)-1H-indol-3-yl]-acetic acid methyl ester.

“Pharmaceutically acceptable salts” refers to the non-toxic, inorganic and organic acid addition salts, and base addition salts, of compounds of the present invention. These salts can be prepared in situ during the final isolation and purification of the compounds.

“Pharmaceutically effective amount” means an amount of compound or compounds according to the present invention effective that produces the desired therapeutic effect described herein, such as allergy relieving, or inflammatory relieving effect.

“Ring group substituent(s)” include alkyl, alkenyl, alkynyl, haloalkyl, haloalkenyl, haloalkynyl, arylalkyl, heteroarylalkyl, acyl, halo, nitro, cyano, hydroxy, alkoxy, alkenyloxy, alkynyloxy, haloalkoxy, haloalkenyloxy, haloalkynyloxy, aryloxy, heteroaryloxy, amino, alkylamino, dialkylamino, arylamino, heteroarylamino, carboxy, alkoxycarbonyl, aryloxycarbonyl, heteroaryloxycarbonyl, arylalkyloxycarbonyl, heteroarylalkyloxycarbonyl, aminocarbonyl, alkylaminocarbonyl, dialkylaminocarbonyl, aroyl, heteroaroyl, cycloalkyl, cycloalkenyl, aryl, heteroaryl, heterocyclyl, heterocyclenyl, or multicyclic alkaryl.

“Solvate” means a physical association of a compound of this invention with one or more solvent molecules. This physical association includes hydrogen bonding. In certain instances the solvate will be capable of isolation, for example when one or more solvent molecules are incorporated in the crystal lattice of the crystalline solid. “Solvate” encompasses both solution-phase and isolable solvates. Representative solvates include hydrates, ethanolates and methanolates.

Some of the compounds of the present invention are basic, and such compounds are useful in the form of the free base, or in the form of a pharmaceutically acceptable acid addition salt thereof.

Acid addition salts are a more convenient form for use; and in practice, use of the salt form inherently amounts to use of the free base form. The acids which can be used to prepare the acid addition salts include preferably those which produce, when combined with the free base, pharmaceutically acceptable salts, that is, salts whose anions are non-toxic to the patient in pharmaceutical doses of the salts, so that the beneficial inhibitory effects inherent in the free base are not vitiated by side effects ascribable to the anions. Although pharmaceutically acceptable salts of said basic compounds are preferred, all acid addition salts are useful as sources of the free base form even if the particular salt, per se, is desired only as an intermediate product as, for example, when the salt is formed only for purposes of purification, and identification, or when it is used as intermediate in preparing a pharmaceutically acceptable salt by ion exchange procedures. In particular, acid addition salts can be prepared by separately reacting the purified compound in its free base form with a suitable organic or inorganic acid and isolating the salt thus formed. Pharmaceutically acceptable salts within the scope of the invention include those derived from mineral acids and organic acids. Exemplary acid addition salts include the hydrobromide, hydrochloride, sulfate, bisulfate, phosphate, nitrate, acetate, oxalate, valerate, oleate, palmitate, quinates, stearate, laurate, borate, benzoate, lactate, phosphate, tosylate, citrate, maleate, fumarate, succinate, tartrate, naphthylate, mesylate, glucoheptonate, lactiobionate, sulfamates, malonates, salicylates, propionates, methylene-bis-β-hydroxynaphthoates, gentisates, isethionates, di-para-toluoyltartrates, ethanesulfonates, benzenesulfonates, cyclohexylsulfamates and laurylsulfonate salts. See, for example S. M. Berge, et al., “Pharmaceutical Salts,” J. Pharm. Sci., 66, 1-19 (1977), which is incorporated herein by reference.

Where the compound of the invention is substituted with an acidic moiety, base addition salts may be formed and are simply a more convenient form for use; and in practice, use of the salt form inherently amounts to use of the free acid form. The bases which can be used to prepare the base addition salts include preferably those which produce, when combined with the free acid, pharmaceutically acceptable salts, that is, salts whose cations are non-toxic to the patient in pharmaceutical doses of the salts, so that the beneficial inhibitory effects inherent in the free base are not vitiated by side effects ascribable to the cations. Base addition salts can also be prepared by separately reacting the purified compound in its acid form with a suitable organic or inorganic base derived from alkali and alkaline earth metal salts and isolating the salt thus formed. Base addition salts include pharmaceutically acceptable metal and amine salts. Suitable metal salts include the sodium, potassium, calcium, barium, zinc, magnesium, and aluminum salts. Particular salts are the sodium and potassium salts. Suitable inorganic base addition salts are prepared from metal bases which include sodium hydride, sodium hydroxide, potassium hydroxide, calcium hydroxide, aluminum hydroxide, lithium hydroxide, magnesium hydroxide, zinc hydroxide and the like. Suitable amine base addition salts are prepared from amines which have sufficient basicity to form a stable salt, and preferably include those amines which are frequently used in medicinal chemistry because of their low toxicity and acceptability for medical use. Ammonia, ethylenediamine, N-methyl-glucamine, lysine, arginine, ornithine, choline, N,N′-dibenzylethylenediamine, chloroprocaine, diethanolamine, procaine, N-benzylphenethylalmine, diethylamine, piperazine, tris(hydroxymethyl)-aminomethane, tetramethylammonium hydroxide, triethylamine, dibenzylainine, ephenamine, dehydroabietylamine, N-ethylpiperidine, benzylamine, tetramethylammonium, tetraethylammonium, methylamine, dimethylamine, trimethylamine, ethylamine, basic amino acids, e.g., lysine and arginine, and dicyclohexylamine.

As well as being useful in themselves as active compounds, salts of compounds of the invention are useful for the purposes of purification of the compounds, for example by exploitation of the solubility differences between the salts and the parent compounds, side products and/or starting materials by techniques well known to those skilled in the art.

It will be appreciated that compounds of the present invention may contain asymmetric centers. These asymmetric centers may independently be in either the R or S configuration. It will be apparent to those skilled in the art that certain compounds of the invention may also exhibit geometrical isomerism. It is to be understood that the present invention includes individual geometrical isomers and stereoisomers and mixtures thereof, including racemic mixtures, of compounds of Formula (XVI) hereinabove. Such isomers can be separated from their mixtures, by the application or adaptation of known methods, for example chromatographic techniques and recrystallization techniques, or they are separately prepared from the appropriate isomers of their intermediates. Additionally, in situations where tautomers of the compounds of Formula (XVI) are possible, the present invention is intended to include all tautomeric forms of the compounds.

### PARTICULAR EMBODIMENTS OF THE INVENTION

One particular embodiment of the invention is a compound of Formula (XVI) wherein n is 1 to 3, or 0 when R³ is carboxy, acid bioisostere, or —C(O)—NY¹Y², or a pharmaceutically acceptable salt, hydrate, or solvate thereof, a pharmaceutically acceptable prodrug thereof, or a pharmaceutically acceptable salt, hydrate or solvate of the prodrug.

One particular embodiment of the invention is a compound of Formula (XVI) wherein n is 1, or a pharmaceutically acceptable salt, hydrate, or solvate thereof, a pharmaceutically acceptable prodrug thereof, or a pharmaceutically acceptable salt, hydrate or solvate of the prodrug.

Another particular embodiment of the invention is a compound of Formula (XVI) wherein the compound is of Formula (I): 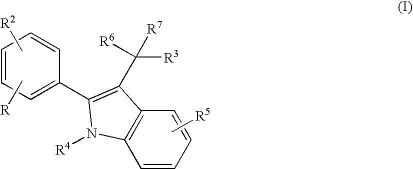
or a pharmaceutically acceptable salt, hydrate, or solvate thereof, a pharmaceutically acceptable prodrug thereof, or a pharmaceutically acceptable salt, hydrate or solvate of the prodrug.

Another particular embodiment of the invention is a compound of Formula (I) wherein:
R is R¹SO₂—, R¹SO—, R¹S—, R⁸—C(═O)—NH— or R⁸—SO₂—NH—;
R¹is alkyl, alkenyl or alkynyl, each of which is optionally substituted by one or more aliphatic group substituents,
   aryl, heteroaryl, or heterocyclyl, each of which is optionally substituted by one or more ring group substituents, or
   —NR′R″ when R is R¹SO₂—;
   R′ is hydrogen,
      aryl, heteroaryl, cycloalkyl, heterocyclyl, arylcycloalkyl, or cycloalkylaryl, each of which is optionally substituted by one or more ring group substituents, or
      alkyl, alkenyl or alknyl, each of which is optionally substituted by one or more aliphatic group substituents;
   R″ is hydrogen, alkyl;
R² is hydrogen, halo, alkyl, alkenyl, alkynyl, haloalkyl, or alkoxy;
R³ is acyl, cyano, carboxy, acid bioisostere, —C(O)—NY¹Y²,
   alkyl, which is optionally substituted by one or more aliphatic group substituents, or alkoxy, which is optionally substituted by one or more aliphatic group substituents,
   Y¹ and Y² are each independently hydrogen, alkylsulfonyl, arylsulfonyl, arylamino, heteroarylsulfonyl, heteroarylamino, or
      alkyl, which is optionally substituted by one or more aliphatic substituent groups;
R⁴ is hydrogen, acyl, alkylsulfonyl, arylsulfonyl, arylalkylsulfonyl, heteroarylsulfonyl, heteroarylalkylsulfonyl, —C(O)—NY⁴Y⁵, —C(O)—O—Y⁶,
   alkyl, alkenyl or alkynyl, each of which is optionally substituted by carboxy, alkoxycarbonyl or acyl, or
   (C₂-C₆)-alkyl, alkenyl or alkynyl, each of which is substituted by hydroxy, alkoxy, amino, alkylamino or dialkylamino;
   Y⁴ and Y⁵ are each independently hydrogen, or alkyl;
   Y⁶ is alkyl;
R⁵ is hydrogen, halo, carboxy, cyano, nitro, hydroxy, alkyl, alkenyl, alkynyl, haloalkyl, haloalkenyl, haloalkynyl, alkoxy, alkenyloxy, alkynyloxy, haloalkoxy, haloalkenyloxy or haloalkynyloxy;
R⁶ and R⁷ are each independently, hydrogen, or alkyl; and
R⁸ is alkyl, which is optionally substituted by one or more aliphatic group substituents, or
   aryl, heteroaryl, cycloalkyl, heterocyclyl, arylcycloalkyl, cycloalkylaryl, heteroarylcycloalkyl, or cycloalkylheteroaryl, each of which is optionally substituted by one or more ring group substituents;
   provided that when R¹ is amino, then R⁴ is hydrogen;
      or a pharmaceutically acceptable salt, hydrate, or solvate thereof, a pharmaceutically acceptable prodrug thereof, or a pharmaceutically acceptable salt, hydrate or solvate of the prodrug.

Another particular embodiment of the invention is a compound of Formula (I) wherein:
R is R¹SO₂—, R⁸—C(═O)—NH— or R⁸—SO₂—NH—;
R¹ is alkyl, aryl, arylalkyl, heteroaryl, heteroarylalkyl, heterocyclyl, or —NR′R″;
   R′ is hydrogen, cycloalkyl, heterocyclyl, arylcycloalkyl, cycloalkylaryl, heteroarylcycloalkyl, cycloalkylheteroaryl,
      aryl or heteroaryl, each of which is optionally substituted by alkyl, halo or haloalkyl, or
      alkyl, which is optionally substituted by cycloalkyl, aryl, or heteroaryl, wherein the cycloalkyl, aryl or heteroaryl is optionally substituted by alkyl, halo or haloalkyl;
   R″ is hydrogen or alkyl;
R² is hydrogen, halo, alkyl, haloalkyl or alkoxy;
R³ is acyl, cyano, carboxy, acid bioisostere, —C(O)—NY¹Y²,
   alkyl, which optionally substituted by hydroxy, alkoxy, amino, alkylamino or dialkylamino, or alkoxy, which is optionally substituted by hydroxy, alkoxy, amino, alkylamino or dialkylamino,
   Y¹ and Y² are each independently hydrogen, alkylsulfonyl, arylsulfonyl, heteroarylsulfonyl, arylamino, heteroarylamino, or
      alkyl, which is optionally substituted by carboxy or alkoxycarbonyl;
R⁴ is hydrogen, acyl, alkylsulfonyl, arylsulfonyl, arylalkylsulfonyl, heteroarylalkyl, heteroarylsulfonyl, heteroarylalkylsulfonyl, arylalkyl, —C(O)—NY⁴Y⁵, —C(O)—O—Y⁶, alkyl, which is optionally substituted by carboxy, alkoxycarbonyl or acyl, or
   (C₂-C₆)-alkyl, which is substituted by hydroxy, alkoxy, amino, alkylamino or dialkylamino;
   Y⁴ and Y⁵ are each independently hydrogen or alkyl;
   Y⁶ is alkyl;
R⁵ is hydrogen, halo, carboxy, cyano, nitro, hydroxy, alkyl, haloalkyl, alkoxy or haloalkoxy;
R⁶ and R⁷ are each independently, hydrogen or alkyl; and
R⁸ is aryl, heteroaryl, cycloalkyl, heterocyclyl, arylcycloalkyl, or cycloalkylaryl;
   provided that when R¹ is amino, then R⁴ is hydrogen;
      or a pharmaceutically acceptable salt, hydrate, or solvate thereof, a pharmaceutically acceptable prodrug thereof, or a pharmaceutically acceptable salt, hydrate or solvate of the prodrug.

Another particular embodiment of the invention is a compound of Formula (I) wherein:
R is R¹SO₂—, R⁸—C(═O)—NH— or R⁸—SO₂—NH—;
R¹ is alkyl, aryl, arylalkyl, heterocyclyl, or —NR′R″;
   R′ is hydrogen, cycloalkyl, heterocyclyl, arylcycloalkyl, cycloalkylaryl,
      aryl, which is optionally substituted by alkyl, halo or haloalkyl, or
      alkyl, which is optionally substituted by cycloalkyl or aryl, wherein the aryl is optionally substituted by alkyl, halo or haloalkyl;
   R″ is hydrogen or alkyl;
R² is hydrogen, halo, alkyl, haloalkyl or alkoxy;
R³ is acyl, cyano, carboxy, acid bioisostere, —C(O)—NY¹Y²,
   alkyl, which optionally substituted by hydroxy, alkoxy, amino, alkylamino or dialkylamino, or alkoxy, which is optionally substituted by hydroxy, alkoxy, amino, alkylamino or dialkylamino,
   Y¹ and Y² are each independently hydrogen, alkylsulfonyl, arylsulfonyl, arylamino, or alkyl, which is optionally substituted by carboxy or alkoxycarbonyl;
R⁴ is hydrogen, acyl, alkylsulfonyl, arylsulfonyl, arylalkylsulfonyl, arylalkyl, —C(O)—NY⁴Y⁵, —C(O)—O—Y⁶
   alkyl, which is optionally substituted by carboxy, alkoxycarbonyl or acyl, or
   (C₂-C₆)-alkyl, which is substituted by hydroxy, alkoxy, amino, alkylamino or dialkylamino;
   Y⁴ and Y⁵ are each independently hydrogen or alkyl;
   Y⁶ is alkyl;
R⁵ is hydrogen, halo, carboxy, cyano, nitro, hydroxy, alkyl, haloalkyl, alkoxy or haloalkoxy;
R⁶ and R⁷ are each independently, hydrogen or alkyl; and
R⁸ is alkyl, aryl, cycloalkyl, heterocyclyl, arylcycloalkyl, or cycloalkylaryl;
   provided that when R¹ is amino, then R⁴ is hydrogen;
      or a pharmaceutically acceptable salt, hydrate, or solvate thereof, a pharmaceutically acceptable prodrug thereof, or a pharmaceutically acceptable salt, hydrate or solvate of the prodrug.

Another particular embodiment of the invention is a compound of Formula (I) wherein R is R¹SO₂—, or a pharmaceutically acceptable salt, hydrate, or solvate thereof, a pharmaceutically acceptable prodrug thereof, or a pharmaceutically acceptable salt, hydrate or solvate of the prodrug.

Another particular embodiment of the invention is a compound of Formula (I) wherein R is R¹SO₂—, and R¹ is —NR′R″, or a pharmaceutically acceptable salt, hydrate, or solvate thereof, a pharmaceutically acceptable prodrug thereof, or a pharmaceutically acceptable salt, hydrate or solvate of the prodrug.

Another particular embodiment of the invention is a compound of Formula (I) wherein:
R is R¹SO₂—;
R¹ is —NR′R″;
R′ is cycloalkyl, heterocyclyl, arylcycloalkyl or cycloalkylaryl; and
R″ is hydrogen or alkyl;
   or a pharmaceutically acceptable salt, hydrate, or solvate thereof, a pharmaceutically acceptable prodrug thereof, or a pharmaceutically acceptable salt, hydrate or solvate of the prodrug.

Another particular embodiment of the invention is a compound of Formula (I) wherein:

R is R¹SO₂—, R¹ is —NR′R″, R′ is cycloalkyl, and R″ is hydrogen or alkyl, or a pharmaceutically acceptable salt, hydrate, or solvate thereof, a pharmaceutically acceptable prodrug thereof, or a pharmaceutically acceptable salt, hydrate or solvate of the prodrug.

Another particular embodiment of the invention is a compound of Formula (I) wherein R is R⁸—SO₂—NH—, or a pharmaceutically acceptable salt, hydrate, or solvate thereof, a pharmaceutically acceptable prodrug thereof, or a pharmaceutically acceptable salt, hydrate or solvate of the prodrug.

Another particular embodiment of the invention is a compound of Formula (I) wherein R² is halo, or a pharmaceutically acceptable salt, hydrate, or solvate thereof, a pharmaceutically acceptable prodrug thereof, or a pharmaceutically acceptable salt, hydrate or solvate of the prodrug.

Another particular embodiment of the invention is a compound of Formula (I) wherein R² is chloro, or a pharmaceutically acceptable salt, hydrate, or solvate thereof, a pharmaceutically acceptable prodrug thereof, or a pharmaceutically acceptable salt, hydrate or solvate of the prodrug.

Another particular embodiment of the invention is a compound of Formula (I) wherein R² is alkyl, alkoxy or haloalkyl, or a pharmaceutically acceptable salt, hydrate, or solvate thereof, a pharmaceutically acceptable prodrug thereof, or a pharmaceutically acceptable salt, hydrate or solvate of the prodrug.

Another particular embodiment of the invention is a compound of Formula (I) wherein R² is methyl, methoxy or —CF₃, or a pharmaceutically acceptable salt, hydrate, or solvate thereof, a pharmaceutically acceptable prodrug thereof, or a pharmaceutically acceptable salt, hydrate or solvate of the prodrug.

Another particular embodiment of the invention is a compound of Formula (I) wherein R³ is —C(O)—NY¹Y², carboxy, acid bioisostere; or alkyl substituted by hydroxy; or a pharmaceutically acceptable salt, hydrate, or solvate thereof, a pharmaceutically acceptable prodrug thereof, or a pharmaceutically acceptable salt, hydrate or solvate of the prodrug.

Another particular embodiment of the invention is a compound of Formula (I) wherein R³ is —COOH, or a pharmaceutically acceptable salt, hydrate, or solvate thereof, a pharmaceutically acceptable prodrug thereof, or a pharmaceutically acceptable salt, hydrate or solvate of the prodrug.

Another particular embodiment of the invention is a compound of Formula (I) wherein R⁴ is hydrogen, alkyl or arylalkyl, or a pharmaceutically acceptable salt, hydrate, or solvate thereof, a pharmaceutically acceptable prodrug thereof, or a pharmaceutically acceptable salt, hydrate or solvate of the prodrug.

Another particular embodiment of the invention is a compound of Formula (I) wherein R⁴ is hydrogen, or a pharmaceutically acceptable salt, hydrate, or solvate thereof, a pharmaceutically acceptable prodrug thereof, or a pharmaceutically acceptable salt, hydrate or solvate of the prodrug.

Another particular embodiment of the invention is a compound of Formula (I) wherein R⁵ is hydrogen, alkyl, alkoxy, hydroxy, halo or haloalkoxy, or a pharmaceutically acceptable salt, hydrate, or solvate thereof, a pharmaceutically acceptable prodrug thereof, or a pharmaceutically acceptable salt, hydrate or solvate of the prodrug.

Another particular embodiment of the invention is a compound of Formula (I) wherein R⁶ and R⁷ are both hydrogen, or a pharmaceutically acceptable salt, hydrate, or solvate thereof, a pharmaceutically acceptable prodrug thereof, or a pharmaceutically acceptable salt, hydrate or solvate of the prodrug.

Another particular embodiment of the invention is a compound of Formula (I) wherein:
R is R¹SO₂—;
R¹ is —NR′R″;
R² is halo;
R³ is —C(O)—NY¹Y², carboxy, acid bioisostere; or alkyl substituted by hydroxy;
R⁴ is hydrogen, alkyl or arylalkyl;
R⁵ is hydrogen, alkyl, alkoxy, hydroxy, halo or haloalkoxy; and
R⁶ and R⁷ are both hydrogen;
   or a pharmaceutically acceptable salt, hydrate, or solvate thereof, a pharmaceutically acceptable prodrug thereof, or a pharmaceutically acceptable salt, hydrate or solvate of the prodrug.

Another particular embodiment of the invention is a compound of Formula (I) wherein:
R is R¹SO₂—;
R¹ is —NR′R″;
   R′ is cycloalkyl, heterocyclyl, arylcycloalkyl, cycloalkylaryl, or alkyl, which is optionally substituted by cycloalkyl or aryl, wherein the aryl is optionally substituted by haloalkyl;
   R″ is hydrogen or alkyl;
R² is halo;
R³ is —C(O)—NY¹Y², carboxy, or acid bioisostere;
   Y¹ and Y² are each independently hydrogen, alkylsulfonyl, arylsulfonyl, or alkyl substituted by carboxy or alkoxycarbonyl;
R⁴ is hydrogen, alkyl or arylalkyl;
R⁵ is hydrogen, alkyl, alkoxy, hydroxy, halo or haloalkoxy; and
R⁶ and R⁷ are both hydrogen;
   or a pharmaceutically acceptable salt, hydrate, or solvate thereof, a pharmaceutically acceptable prodrug thereof, or a pharmaceutically acceptable salt, hydrate or solvate of the prodrug.

Another particular embodiment of the invention is a compound of Formula (I) wherein:
R is R¹SO₂—;
R¹ is piperidinyl, or —NR′R″;
   R′ is hydrogen, cycloheptane, cycloheptane-methylene, cyclohexane, cyclohexane-methylene, cyclohexane-ethylene, cyclopentane, bicyclo[2.2.1]heptane, indanyl, phenyl, tetrahydropyranyl, tricyclo[3.3.1.13.7]decane-methylene, methyl, isopropyl, isopentyl, n-hexanyl, benzyl, or 4-trifluoromethyl-benzyl;
   R″ is hydrogen or methyl;
R² is chloro;
R³ is carboxy, —CH₂—OH, —C(O)—NH₂, —C(═O)—NH—SO₂—CH₃, 5-oxo-4,5-dihydro-1,3,4-oxadiazol-2-yl, 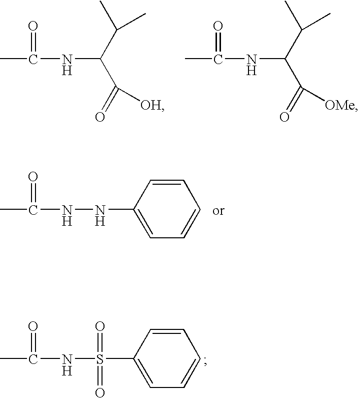
R⁴ is hydrogen, methyl or benzyl;
R⁵ is hydrogen, chloro, hydroxy, methyl, isopropyl, t-butyl, methoxy or trifluoromethoxy; and
R⁶ and R⁷ are both hydrogen;
   or a pharmaceutically acceptable salt, hydrate, or solvate thereof, a pharmaceutically acceptable prodrug thereof, or a pharmaceutically acceptable salt, hydrate or solvate of the prodrug.

Another particular embodiment of the invention is a compound of Formula (I) wherein:
R is R¹SO₂—;
R¹ is —NR′R″;
   R′ is cycloheptane, cycloheptane-methylene, cyclohexane, cyclohexane-methylene, cyclohexane-ethylene, cyclopentane, bicyclo[2.2.1]heptane, indanyl, tetrahydropyranyl,
      tricyclo[3.3.1.13.7]decane-methylene, isopropyl, isopentyl, n-hexanyl, benzyl, or 4-trifluoromethyl-benzyl;
   R″ is hydrogen or methyl;
R² is chloro;
R³ is carboxy, —C(O)—NH₂, 5-oxo-4,5-dihydro-1,3,4-oxadiazol-2-yl, 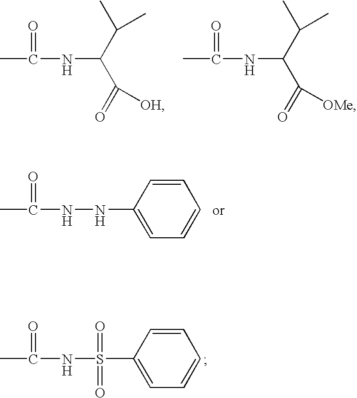
R⁴ is hydrogen, methyl or benzyl;
R⁵ is hydrogen, chloro, hydroxy, methyl, isopropyl, t-butyl, methoxy or trifluoromethoxy; and
R⁶ and R⁷ are both hydrogen;
   or a pharmaceutically acceptable salt, hydrate, or solvate thereof, a pharmaceutically acceptable prodrug thereof, or a pharmaceutically acceptable salt, hydrate or solvate of the prodrug.

Another particular embodiment of the invention is a compound of Formula (I) wherein the compound is of Formula (II): 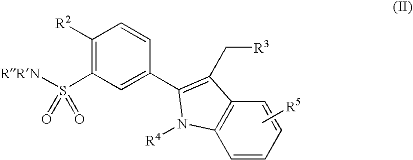
or a pharmaceutically acceptable salt, hydrate, or solvate thereof, a pharmaceutically acceptable prodrug thereof, or a pharmaceutically acceptable salt, hydrate or solvate of the prodrug.

Another particular embodiment of the invention is a compound of Formula (II) wherein R′ is cycloalkyl, heterocyclyl, arylcycloalkyl or cycloalkylaryl, and R″ is hydrogen or alkyl; or a pharmaceutically acceptable salt, hydrate, or solvate thereof, a pharmaceutically acceptable prodrug thereof, or a pharmaceutically acceptable salt, hydrate or solvate of the prodrug.

Another particular embodiment of the invention is a compound of Formula (II) wherein R′ is cycloalkyl, and R″ is hydrogen or alkyl; or a pharmaceutically acceptable salt, hydrate, or solvate thereof, a pharmaceutically acceptable prodrug thereof, or a pharmaceutically acceptable salt, hydrate or solvate of the prodrug.

Another particular embodiment of the invention is a compound of Formula (II) wherein R² is halo, or a pharmaceutically acceptable salt, hydrate, or solvate thereof, a pharmaceutically acceptable prodrug thereof, or a pharmaceutically acceptable salt, hydrate or solvate of the prodrug.

Another particular embodiment of the invention is a compound of Formula (II) wherein R² is chloro, or a pharmaceutically acceptable salt, hydrate, or solvate thereof, a pharmaceutically acceptable prodrug thereof, or a pharmaceutically acceptable salt, hydrate or solvate of the prodrug.

Another particular embodiment of the invention is a compound of Formula (II) wherein R² is alkyl, alkoxy or haloalkyl, or a pharmaceutically acceptable salt, hydrate, or solvate thereof, a pharmaceutically acceptable prodrug thereof, or a pharmaceutically acceptable salt, hydrate or solvate of the prodrug.

Another particular embodiment of the invention is a compound of Formula (II) wherein R² is methyl, methoxy or —CF₃, or a pharmaceutically acceptable salt, hydrate, or solvate thereof, a pharmaceutically acceptable prodrug thereof, or a pharmaceutically acceptable salt, hydrate or solvate of the prodrug.

Another particular embodiment of the invention is a compound of Formula (II) wherein R³ is —C(O)—NY¹Y², carboxy, acid bioisostere; or alkyl substituted by hydroxy; or a pharmaceutically acceptable salt, hydrate, or solvate thereof, a pharmaceutically acceptable prodrug thereof, or a pharmaceutically acceptable salt, hydrate or solvate of the prodrug.

Another particular embodiment of the invention is a compound of Formula (II) wherein R³ is —COOH, or a pharmaceutically acceptable salt, hydrate, or solvate thereof, a pharmaceutically acceptable prodrug thereof, or a pharmaceutically acceptable salt, hydrate or solvate of the prodrug.

Another particular embodiment of the invention is a compound of Formula (II) wherein R⁴ is hydrogen, alkyl or arylalkyl, or a pharmaceutically acceptable salt, hydrate, or solvate thereof, a pharmaceutically acceptable prodrug thereof, or a pharmaceutically acceptable salt, hydrate or solvate of the prodrug.

Another particular embodiment of the invention is a compound of Formula (II) wherein R⁴ is hydrogen, or a pharmaceutically acceptable salt, hydrate, or solvate thereof, a pharmaceutically acceptable prodrug thereof, or a pharmaceutically acceptable salt, hydrate or solvate of the prodrug.

Another particular embodiment of the invention is a compound of Formula (II) wherein R⁵ is hydrogen, alkyl, alkoxy, hydroxy, halo or haloalkoxy, or a pharmaceutically acceptable salt, hydrate, or solvate thereof, a pharmaceutically acceptable prodrug thereof, or a pharmaceutically acceptable salt, hydrate or solvate of the prodrug.

Another particular embodiment of the invention is a compound of Formula (II) wherein:
R′ is cycloalkyl, heterocyclyl, arylcycloalkyl, cycloalkylaryl, or
   alkyl, optionally substituted by cycloalkyl or aryl, wherein the aryl is optionally substituted by haloalkyl;
R″ is hydrogen or alkyl;
R² is halo;
R³ is —C(O)—NY¹Y², carboxy, acid bioisostere; or alkyl substituted by hydroxy;
R⁴ is hydrogen, alkyl or arylalkyl; and
R⁵ is hydrogen, alkyl, alkoxy, hydroxy, halo or haloalkoxy,
   or a pharmaceutically acceptable salt, hydrate, or solvate thereof, a pharmaceutically acceptable prodrug thereof, or a pharmaceutically acceptable salt, hydrate or solvate of the prodrug.

Another particular embodiment of the invention is a compound of Formula (II) wherein:
R′ is cycloalkyl, heterocyclyl, arylcycloalkyl or cycloalkylaryl, or alkyl or alkyl substituted by cycloalkyl;
R″ is hydrogen or alkyl;
R² is halo;
R³ is —C(O)—NY¹Y², carboxy, or acid bioisostere;
   Y¹ and Y² are each independently hydrogen, alkylsulfonyl, arylsulfonyl, or alkyl substituted by carboxy or alkoxycarbonyl;
R⁴ is hydrogen, alkyl or arylalkyl; and
R⁵ is hydrogen, alkyl, alkoxy, hydroxy, halo or haloalkoxy,
   or a pharmaceutically acceptable salt, hydrate, or solvate thereof, a pharmaceutically acceptable prodrug thereof, or a pharmaceutically acceptable salt, hydrate or solvate of the prodrug.

Another particular embodiment of the invention is a compound of Formula (II) wherein:
R′ is hydrogen, cycloheptane, cycloheptane-methylene, cyclohexane, cyclohexane-methylene, cyclohexane-ethylene, cyclopentane, bicyclo[2.2.1]heptane, indanyl, phenyl, tetrahydropyranyl, tricyclo[3.3.1.13.7]decane-methylene, methyl, isopropyl, isopentyl, n-hexanyl, benzyl or 4-trifluoromethyl-benzyl;
R″ is hydrogen or methyl;
R² is chloro;
R³ is carboxy, —CH₂—OH, —C(O)—NH₂, —C(═O)—NH—SO₂—CH₃, 5-oxo-4,5-dihydro-1,3,4-oxadiazol-2-yl, 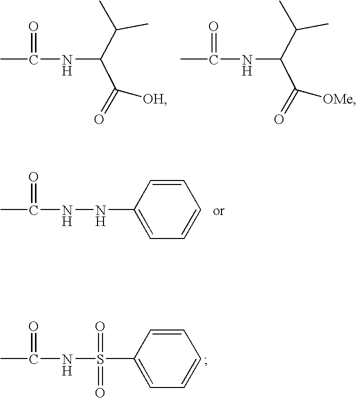
R⁴ is hydrogen, methyl or benzyl; and
R⁵ is hydrogen, chloro, hydroxy, methyl, isopropyl, t-butyl, methoxy or trifluoromethoxy,
   or a pharmaceutically acceptable salt, hydrate, or solvate thereof, a pharmaceutically acceptable prodrug thereof, or a pharmaceutically acceptable salt, hydrate or solvate of the prodrug.

Another particular embodiment of the invention is a compound of Formula (II) wherein:
R′ is cycloheptane, cycloheptane-methylene, cyclohexane, cyclohexane-methylene, cyclohexane-ethylene, cyclopentane, bicyclo[2.2.1]heptane, indanyl, tetrahydropyranyl, tricyclo[3.3.1.13.7]decane-methylene, isopropyl, isopentyl, n-hexanyl, benzyl or 4-trifluoromethyl-benzyl;
R″ is hydrogen or methyl;
R² is chloro;
R³ is carboxy, —C(O)—NH₂, 5-oxo-4,5-dihydro-1,3,4-oxadiazol-2-yl, 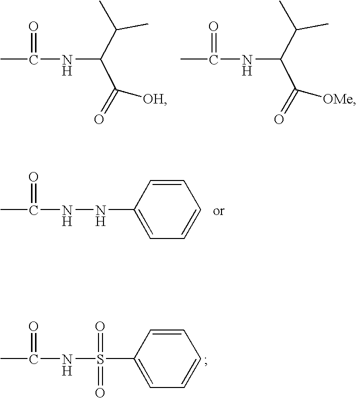
R⁴ is hydrogen, methyl or benzyl; and
R⁵ is hydrogen, chloro, hydroxy, methyl, isopropyl, t-butyl, methoxy or trifluoromethoxy,
   or a pharmaceutically acceptable salt, hydrate, or solvate thereof, a pharmaceutically acceptable prodrug thereof, or a pharmaceutically acceptable salt, hydrate or solvate of the prodrug.

Another particular embodiment of the invention is a compound of Formula (XVI) or a pharmaceutically acceptable ester prodrug thereof, which is
[2-(4-Chloro-3-cyclohexylsulfamoyl-phenyl)-1H-indol-3-yl]-acetic acid, Example 1(a);
{2-[3-(Bicyclo[2.2.1]hept-2-ylsulfamoyl)-4-chloro-phenyl]-1H-indol-3-yl}-acetic acid, Example 1(b);
[2-(4-Chloro-3-hexylsulfamoyl-phenyl)-1H-indol-3-yl]-acetic acid, Example 1(c);
{2-[4-Chloro-3-(indan-2-ylsulfamoyl)-phenyl]-1H-indol-3-yl}-acetic acid, Example 1(d);
[2-(4-Chloro-3-cyclopentylsulfamoyl-phenyl)-1H-indol-3-yl]-acetic acid, Example 1 (e);
{2-[4-Chloro-3-(2,2-dimethyl-propylsulfamoyl)-phenyl]-1H-indol-3-yl}-acetic acid, Example (f);
[2-(4-Chloro-3-isopropylsulfamoyl-phenyl)-1H-indol-3-yl]-acetic acid, Example 1 (g);
{2-[4-Chloro-3-(2-cyclohexyl-ethylsulfamoyl)-phenyl]-1H-indol-3-yl}-acetic acid, Example 1(h);
[2-(4-Chloro-3-phenylsulfamoyl-phenyl)-1H-indol-3-yl]-acetic acid, Example 1(i);
{2-[4-Chloro-3-(cyclohexylmethyl-sulfamoyl)-phenyl]-1H-indol-3-yl}-acetic acid, Example 1(j);
{2-[4-Chloro-3-(1-ethyl-propylsulfamoyl)-phenyl]-1H-indol-3-yl}-acetic acid, Example 1(k);
{2-[4-Chloro-3-(cycloheptylmethyl-sulfamoyl)-phenyl]-1H-indol-3-yl}-acetic acid, Example 1(l);
(2-{4-Chloro-3-[(tricyclo[3.3.1.13.7]decan-1-ylmethyl)-sulfamoyl]-phenyl}-1H-indol-3-yl)-acetic acid, Example 1(m);
[2-(4-Chloro-3-cycloheptylsulfamoyl-phenyl)-1H-indol-3-yl]-acetic acid, Example 1(n);
{2-[4-Chloro-3-(tetrahydro-pyran-4-ylsulfamoyl)-phenyl]-1H-indol-3-yl}-acetic acid, Example 1(o);
{2-[4-Chloro-3-(piperidine-1-sulfonyl)-phenyl]-1H-indol-3-yl}-acetic acid, Example 1(p);
[2-(4-Chloro-3-methylsulfamoyl-phenyl)-1H-indol-3-yl]-acetic acid, Example 1(q);
[2-(4-Chloro-3-sulfamoyl-phenyl)-1H-indol-3-yl]-acetic acid, Example 1(r);
[5-tert-Butyl-2-(4-chloro-3-cyclohexylsulfamoyl-phenyl)-1H-indol-3-yl]-acetic acid, Example 1(s);
[2-(4-Chloro-3-cyclohexylsulfamoyl-phenyl)-5-methyl-1H-indol-3-yl]-acetic acid, Example 1(t);
[2-(4-Chloro-3-cyclohexylsulfamoyl-phenyl)-5-isopropyl-1H-indol-3-yl]-acetic acid, Example 1(u);
[2-(4-Chloro-3-cyclohexylsulfamoyl-phenyl)-5-trifluoromethoxy-1H-indol-3-yl]-acetic acid, Example 1(v);
[2-(3-Benzylsulfamoyl-4-chloro-phenyl)-1H-indol-3-yl]-acetic acid, Example 1(w);
{2-[4-Chloro-3-(cyclohexyl-methyl-sulfamoyl)-phenyl]-1H-indol-3-yl}-acetic acid, Example 1(x);
{2-[4-Chloro-3-(4-trifluoromethyl-benzylsulfamoyl)-phenyl]-1H-indol-3-yl}-acetic acid, Example 1(y);
[2-(4-Chloro-3-cyclohexylsulfamoyl-phenyl)-1-methyl-1H-indol-3-yl]-acetic acid, Example 2(a);
[1-Benzyl-2-(4-chloro-3-cyclohexylsulfamoyl-phenyl)-1H-indol-3-yl]-acetic acid, Example 2(b);
{2-[4-Chloro-3-(piperidine-1-sulfonyl)-phenyl]-1-methyl-1H-indol-3-yl}-acetic acid, Example 2(c);
(S)-2-{2-[2-(4-Chloro-3-cyclohexylsulfamoyl-phenyl)-1H-indol-3-yl]-acetylamino}-3-methyl-butyric acid, Example 3(a);
(S)-2-{2-[2-(4-Chloro-3-cyclohexylsulfamoyl-phenyl)-1H-indol-3-yl]-acetylamino}-3-methyl-butyric acid, Example 3(b);
[2-(4-Chloro-3-cyclohexylsulfamoyl-phenyl)-1H-indol-3-yl]-acetic acid 2-dimethylamino-ethyl ester, Example 4;
2-Chloro-N-cyclohexyl-5-[3-(5-oxo-4,5-dihydro-1,3,4-oxadiazol-2-ylmethyl)-1H-indol-2-yl]-benzenesulfonamide, Example 5;
5-[3-(2-Benzenesulfonylamino-2-oxo-ethyl)-1H-indol-2-yl]-2-chloro-N-cyclohexyl benzenesulfonamide, Example 6;
2-[2-(4-Chloro-3-cyclohexylsulfamoyl-phenyl)-1H-indol-3-yl]-acetamide, Example 7(a);
2-[2-(4-Chloro-3-cyclohexylsulfamoyl-phenyl)-1-methyl-1H-indol-3-yl]-acetamide, Example 7(b);
[2-(4-Chloro-3-cyclohexylsulfamoyl-phenyl)-1H-indol-3-yl]-acetic acid methyl ester, Example 8;
2-Chloro-N-cyclohexyl-5-[3-(2-hydroxy-ethyl)-1-methyl-1H-indol-2-yl]-benzenesulfonamide, Example 9;
[2-(4-Chloro-3-cyclohexylsulfamoyl-phenyl)-5-methoxy-1H-indol-3-yl]-acetic acid, Example 10(a);
[5-Chloro-2-(4-chloro-3-cyclohexylsulfamoyl-phenyl)-1H-indol-3-yl]-acetic acid, Example 10(b);
[2-(4-Chloro-3-cyclohexylsulfamoyl-phenyl)-5-hydroxy-1H-indol-3-yl]-acetic acid, Example 10(c);
[6-Chloro-2-(4-chloro-3-cyclohexylsulfamoyl-phenyl)-1H-indol-3-yl]-acetic acid, Example 10(d);
{2-[3-(Cyclohexyl-methyl-sulfamoyl)-phenyl]-1H-indol-3-yl}-acetic acid, Example 10(e);
[2-(3-Cyclohexylsulfamoyl-phenyl)-1H-indol-3-yl]-acetic acid, Example 10(f);
2-[2-(3-Cyclohexylsulfamoyl-phenyl)-1H-indol-3-yl]-propionic acid, Example 10(g);
[2-(4-Cyclohexylsulfamoyl-phenyl)-1H-indol-3-yl]-acetic acid, Example 10(h);
[2-(3-Cyclohexylsulfamoyl-4-methoxy-phenyl)-1H-indol-3-yl]-acetic acid, Example 10(i);
[2-(3-Chloro-4-cyclohexylsulfamoyl-phenyl)-1H-indol-3-yl]-acetic acid, Example 10(j);
[2-(3-Cyclohexylsulfamoyl-4-methyl-phenyl)-1H-indol-3-yl]-acetic acid, Example 10(k);
[2-(3-Cyclohexylsulfamoyl-5-trifluoromethyl-phenyl)-1H-indol-3-yl]-acetic acid methyl ester, Example 11;
[2-(3-Cyclohexylsulfamoyl-5-trifluoromethyl-phenyl)-1H-indol-3-yl]-acetic acid, Example 12;
[2-(3-Benzenesulfonylamino-4-chlorophenyl)-1H-indol-3-yl]-acetic acid, Example 13;
{2-[4-Chloro-3-(cyclohexanecarbonyl-amino)-phenyl]-1H-indol-3-yl}-acetic acid, Example 14;
2-(4-Chloro-3-cyclohexylsulfamoyl-phenyl)-1H-indole-3-carboxylic acid, Example 15; or
2-(4-Chloro-3-cyclohexylsulfamoyl-phenyl)-1H-indole-6-carboxylic acid, Example 16;
   or a pharmaceutically acceptable ester prodrug thereof.

It is to be understood that this invention covers all appropriate combinations of the particular embodiments referred thereto.

The compounds of present invention and the intermediates and starting materials used in their preparation are named in accordance with IUPAC rules of nomenclature in which the characteristic groups have decreasing priority for citation as the principle group as follows: acids, esters, amides, etc. However, it is understood that, for a particular compound referred to by both a structural Formula and a nomenclature name, if the structural Formula and the nomenclature name are inconsistent with each other, the structural Formula takes the precedence over the nomenclature name.

The compounds of the invention exhibit prostaglandin D2 receptor antagonist activity and are useful as pharmacological acting agents. Accordingly, they are incorporated into pharmaceutical compositions and used in the treatment of patients suffering from certain medical disorders.

Compounds within the scope of the present invention are antagonists of the prostaglandin D2 receptor, according to tests described in the literature and described in pharmacological testing section hereinafter, and which tests results are believed to correlate to pharmacological activity in humans and other mammals. Thus, in a further embodiment, the present invention provides compounds of the invention and compositions containing compounds of the invention for use in the treatment of a patient suffering from, or subject to, conditions, which can be ameliorated by the administration of a PGD2 antagonist. For example, compounds of the present invention could therefore be useful in the treatment of a variety of PGD2-mediated disorders including, but not limited to, allergic disease (such as allergic rhinitis, allergic conjunctivitis, atopic dermatitis, bronchial asthma and food allergy), systemic mastocytosis, disorders accompanied by systemic mast cell activation, anaphylaxis shock, bronchoconstriction, bronchitis, urticaria, eczema, diseases accompanied by itch (such as atopic dermatitis and urticaria), diseases (such as cataract, retinal detachment, inflammation, infection and sleeping disorders) which are generated secondarily as a result of behavior accompanied by itch (such as scratching and beating), inflammation, chronic obstructive pulmonary diseases, ischemic reperfusion injury, cerebrovascular accident, chronic rheumatoid arthritis, pleurisy, ulcerative colitis and the like.

Compounds of the present invention are further useful in treatments involving a combination therapy with:

(i) antihistamines, such as fexofenadine, loratadine and citirizine, for the treatment of allergic rhinitis;

(ii) leukotriene antagonists, such as montelukast and zafirlukast, for the treatment of allergic rhinitis, COPD, allergic dermatitis, allergic conjunctivitis, etc—please specifically refer to the claims in WO 01/78697 A2;

(iii) beta agonists, such as albuterol, salbuterol and terbutaline, for the treatment of asthma, COPD, allergic dermatitis, allergic conjunctivitis, etc;

(iv) antihistamines, such as fexofenadine, loratadine and citirizine, for the treatment of asthma, COPD, allergic dermatitis, allergic conjunctivitis, etc;

(v) PDE4 (Phosphodiesterase 4) inhibitors, such as roflumilast and cilomilast, for the treatment of asthma, COPD, allergic dermatitis, allergic conjunctivitis, etc; or

(vi) with TP (Thromboxane A2 receptor) or CrTh2 (chemoattractant receptor-homologous molecule expressed on Th2 cells) antagonists, such as Ramatrobran (BAY-u3405), for the treatment of COPD, allergic dermatitis, allergic conjunctivitis, etc.

A special embodiment of the therapeutic methods of the present invention is the treating of allergic rhinitis.

Another special embodiment of the therapeutic methods of the present invention is the treating of bronchial asthma.

According to a further feature of the invention there is provided a method for the treatment of a human, or animal patient suffering from, or subject to, conditions which can be ameliorated by the administration of a prostaglandin D2 receptor antagonist, for example conditions as hereinbefore described, which comprises the administration to the patient of an effective amount of compound of the invention or a composition containing a compound of the invention. “Effective amount” is meant to describe an amount of compound of the present invention effective as a prostaglandin D2 receptor antagonist and thus producing the desired therapeutic effect.

References herein to treatment should be understood to include prophylactic therapy as well as treatment of established conditions.

The present invention also includes within its scope pharmaceutical compositions comprising at least one of the compounds of the invention in admixture with a pharmaceutically acceptable carrier.

In practice, the compound of the present invention may be administered in pharmaceutically acceptable dosage form to humans and other animals by topical or systemic administration, including oral, inhalational, rectal, nasal, buccal, sublingual, vaginal, colonic, parenteral (including subcutaneous, intramuscular, intravenous, intradermal, intrathecal and epidural), intracisternal and intraperitoneal. It will be appreciated that the preferred route may vary with for example the condition of the recipient.

“Pharmaceutically acceptable dosage forms” refers to dosage forms of the compound of the invention, and includes, for example, tablets, dragées, powders, elixirs, syrups, liquid preparations, including suspensions, sprays, inhalants tablets, lozenges, emulsions, solutions, granules, capsules and suppositories, as well as liquid preparations for injections, including liposome preparations. Techniques and formulations generally may be found in Remington's Pharmaceutical Sciences, Mack Publishing Co., Easton, Pa., latest edition.

A particular aspect of the invention provides for a compound according to the present invention to be administered in the form of a pharmaceutical composition. Pharmaceutical compositions, according to the present invention, comprise compounds of the present invention and pharmaceutically acceptable carriers.

Pharmaceutically acceptable carriers include at least one component selected from the group comprising pharmaceutically acceptable carriers, diluents, coatings, adjuvants, excipients, or vehicles, such as preserving agents, fillers, disintegrating agents, wetting agents, emulsifying agents, emulsion stabilizing agents, suspending agents, isotonic agents, sweetening agents, flavoring agents, perfuming agents, coloring agents, antibacterial agents, antifungal agents, other therapeutic agents, lubricating agents, adsorption delaying or promoting agents, and dispensing agents, depending on the nature of the mode of administration and dosage forms.

Exemplary suspending agents include ethoxylated isostearyl alcohols, polyoxyethylene sorbitol and sorbitan esters, microcrystalline cellulose, aluminum metahydroxide, bentonite, agar-agar and tragacanth, or mixtures of these substances.

Exemplary antibacterial and antifungal agents for the prevention of the action of microorganisms include parabens, chlorobutanol, phenol, sorbic acid, and the like.

Exemplary isotonic agents include sugars, sodium chloride, and the like.

Exemplary adsorption delaying agents to prolong absorption include aluminum monostearate and gelatin.

Exemplary adsorption promoting agents to enhance absorption include dimethyl sulfoxide and related analogs.

Exemplary diluents, solvents, vehicles, solubilizing agents, emulsifiers and emulsion stabilizers, include water, chloroform, sucrose, ethanol, isopropyl alcohol, ethyl carbonate, ethyl acetate, benzyl alcohol, tetrahydrofurfuryl alcohol, benzyl benzoate, polyols, propylene glycol, 1,3-butylene glycol, glycerol, polyethylene glycols, dimethylformamide, Tween®60, Span® 60, cetostearyl alcohol, myristyl alcohol, glyceryl mono-stearate and sodium lauryl sulfate, fatty acid esters of sorbitan, vegetable oils (such as cottonseed oil, groundnut oil, com germ oil, olive oil, castor oil and sesame oil) and injectable organic esters such as ethyl oleate, and the like, or suitable mixtures of these substances.

Exemplary excipients include lactose, milk sugar, sodium citrate, calcium carbonate and dicalcium phosphate.

Exemplary disintegrating agents include starch, alginic acids and certain complex silicates.

Exemplary lubricants include magnesium stearate, sodium lauryl sulfate, talc, as well as high molecular weight polyethylene glycols.

The choice of pharmaceutical acceptable carrier is generally determined in accordance with the chemical properties of the active compound such as solubility, the particular mode of administration and the provisions to be observed in pharmaceutical practice.

Pharmaceutical compositions of the present invention suitable for oral administration may be presented as discrete units such as a solid dosage form, such as capsules, cachets or tablets each containing a predetermined amount of the active ingredient, or as a powder or granules; as a liquid dosage form such as a solution or a suspension in an aqueous liquid or a non-aqueous liquid, or as an oil-in-water liquid emulsion or a water-in-oil liquid emulsion. The active ingredient may also be presented as a bolus, electuary or paste.

“Solid dosage form” means the dosage form of the compound of the invention is solid form, for example capsules, tablets, pills, powders, dragées or granules. In such solid dosage forms, the compound of the invention is admixed with at least one inert customary excipient (or carrier) such as sodium citrate or dicalcium phosphate or (a) fillers or extenders, as for example, starches, lactose, sucrose, glucose, mannitol and silicic acid, (b) binders, as for example, carboxymethylcellulose, alginates, gelatin, polyvinylpyrrolidone, sucrose and acacia, (c) humectants, as for example, glycerol, (d) disintegrating agents, as for example, agar-agar, calcium carbonate, potato or tapioca starch, alginic acid, certain complex silicates and Na₂CO₃, (e) solution retarders, as for example paraffin, (f) absorption accelerators, as for example, quaternary ammonium compounds, (g) wetting agents, as for example, cetyl alcohol and glycerol monostearate, (h) adsorbents, as for example, kaolin and bentonite, (i) lubricants, as for example, talc, calcium stearate, magnesium stearate, solid polyethylene glycols, sodium lauryl sulfate, (j) opacifying agents, (k) buffering agents, and agents which release the compound(s) of the invention in a certain part of the intestinal tract in a delayed manner.

A tablet may be made by compression or molding, optionally with one or more accessory ingredients. Compressed tables may be prepared by compressing in a suitable machine the active ingredient in a free-flowing form such as a powder or granules, optionally mixed with a binder, lubricant, inert diluent, preservative, surface active or dispersing agent. Excipients such as lactose, sodium citrate, calcium carbonate, dicalcium phosphate and disintegrating agents such as starch, alginic acids and certain complex silicates combined with lubricants such as magnesium stearate, sodium lauryl sulfate and talc may be used. A mixture of the powdered compounds moistened with an inert liquid diluent may be molded in a suitable machine to make molded tablets. The tablets may optionally be coated or scored and may be formulated so as to provide slow or controlled release of the active ingredient therein.

Solid compositions may also be employed as fillers in soft and hard-filled gelatin capsules using such excipients as lactose or milk sugar as well as high molecular weight polyethylene glycols, and the like.

If desired, and for more effective distribution, the compounds can be microencapsulated in, or attached to, a slow release or targeted delivery systems such as a biocompatible, biodegradable polymer matrices (e.g., poly(d,l-lactide co-glycolide)), liposomes, and microspheres and subcutaneously or intramuscularly injected by a technique called subcutaneous or intramuscular depot to provide continuous slow release of the compound(s) for a period of 2 weeks or longer. The compounds may be sterilized, for example, by filtration through a bacteria-retaining filter, or by incorporating sterilizing agents in the form of sterile solid compositions that can be dissolved in sterile water, or some other sterile injectable medium immediately before use.

“Liquid dosage form” means the dose of the active compound to be administered to the patient is in liquid form, for, example, pharmaceutically acceptable emulsions, solutions, suspensions, syrups and elixirs. In addition to the active compounds, the liquid dosage forms may contain inert diluents commonly used in the art, such solvents, solubilizing agents and emulsifiers.

When aqueous suspensions are used they can contain emulsifying agents or agents which facilitate suspension.

Pharmaceutical compositions suitable for topical administration means formulations that are in a form suitable to be administered topically to a patient. The formulation may be presented as a topical ointment, salves, powders, sprays and inhalants, gels (water or alcohol based), creams, as is generally known in the art, or incorporated into a matrix base for application in a patch, which would allow a controlled release of compound through the transdermal barrier. When formulated in an ointment, the active ingredients may be employed with either a paraffinic or a water-miscible ointment base. Alternatively, the active ingredients may be formulated in a cream with an oil-in-water cream base. Formulations suitable for topical administration in the eye include eye drops wherein the active ingredient is dissolved or suspended in a suitable carrier, especially an aqueous solvent for the active ingredient. Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

The oily phase of the emulsion pharmaceutical composition may be constituted from known ingredients in a known manner. While the phase may comprise merely an emulsifier (otherwise known as an emulgent), it desirably comprises a mixture of at least one emulsifier with a fat or an oil or with both a fat and an oil. In a particular embodiment, a hydrophilic emulsifier is included together with a lipophilic emulsifier that acts as a stabilizer. Together, the emulsifier(s) with or without stabilizer(s) make up the emulsifying wax, and the way together with the oil and fat make up the emulsifying ointment base which forms the oily dispersed phase of the cream formulations.

If desired, the aqueous phase of the cream base may include, for example, a least 30% w/w of a polyhydric alcohol, i.e. an alcohol having two or more hydroxy groups such as propylene glycol, butane 1,3-diol, mannitol, sorbitol, glycerol and polyethylene glycol (including PEG 400) and mixtures thereof. The topical formulations may desirably include a compound that enhances absorption or penetration of the active ingredient through the skin or other affected areas.

The choice of suitable oils or fats for a composition is based on achieving the desired properties. Thus a cream should preferably be a non-greasy, non-staining and washable product with suitable consistency to avoid leakage from tubes or other containers. Straight or branched chain, mono- or dibasic alkyl esters such as di-isopropyl myristate, decyl oleate, isopropyl palmitate, butyl stearate, 2-ethylhexyl palmitate or a blend of branched chain esters known as Crodamol CAP may be used. These may be used alone or in combination depending on the properties required. Alternatively, high melting point lipids such as white soft paraffin and/or liquid paraffin or other mineral oils can be used.

Pharmaceutical compositions suitable for rectal or vaginal administrations means formulations that are in a form suitable to be administered rectally or vaginally to a patient and containing at least one compound of the invention. Suppositories are a particular form for such formulations that can be prepared by mixing the compounds of this invention with suitable non-irritating excipients or carriers such as cocoa butter, polyethylene glycol or a suppository wax, which are solid at ordinary temperatures but liquid at body temperature and therefore, melt in the rectum or vaginal cavity and release the active component.

Pharmaceutical composition administered by injection may be by transmuscular, intravenous, intraperitoneal, and/or subcutaneous injection. The compositions of the present invention are formulated in liquid solutions, in particular in physiologically compatible buffers such as Hank's solution or Ringer's solution. In addition, the compositions may be formulated in solid form and redissolved or suspended immediately prior to use. Lyophilized forms are also included. The formulations are sterile and include emulsions, suspensions, aqueous and non-aqueous injection solutions, which may contain suspending agents and thickening agents and anti-oxidants, buffers, bacteriostats and solutes which render the formulation isotonic, and have a suitably adjusted pH, with the blood of the intended recipient.

Pharmaceutical composition of the present invention suitable for nasal or inhalational administration means compositions that are in a form suitable to be administered nasally or by inhalation to a patient. The composition may contain a carrier, in a powder form, having a particle size for example in the range 1 to 500 microns (including particle sizes in a range between 20 and 500 microns in increments of 5 microns such as 30 microns, 35 microns, etc.). Suitable compositions wherein the carrier is a liquid, for administration as for example a nasal spray or as nasal drops, include aqueous or oily solutions of the active ingredient. Compositions suitable for aerosol administration may be prepared according to conventional methods and may be delivered with other therapeutic agents. Metered dose inhalers are useful for administering compositions according to the invention for an inhalational therapy.

Actual dosage levels of active ingredient(s) in the compositions of the invention may be varied so as to obtain an amount of active ingredient(s) that is (are) effective to obtain a desired therapeutic response for a particular composition and method of administration for a patient. A selected dosage level for any particular patient therefore depends upon a variety of factors including the desired therapeutic effect, on the route of administration, on the desired duration of treatment, the etiology and severity of the disease, the patient's condition, weight, sex, diet and age, the type and potency of each active ingredient, rates of absorption, metabolism and/or excretion and other factors.

Total daily dose of the compounds of this invention administered to a patient in single or divided doses may be in amounts, for example, of from about 0.001 to about 100 mg/kg body weight daily and preferably 0.01 to 10 mg/kg/day. For example, in an adult, the doses are generally from about 0.01 to about 100, preferably about 0.01 to about 10, mg/kg body weight per day by inhalation, from about 0.01 to about 100, preferably 0.1 to 70, more especially 0.5 to 10, mg/kg body weight per day by oral administration, and from about 0.01 to about 50, preferably 0.01 to 10, mg/kg body weight per day by intravenous administration. The percentage of active ingredient in a composition may be varied, though it should constitute a proportion such that a suitable dosage shall be obtained. Dosage unit compositions may contain such amounts of such submultiples thereof as may be used to make up the daily dose. Obviously, several unit dosage forms may be administered at about the same time. A dosage may be administered as frequently as necessary in order to obtain the desired therapeutic effect. Some patients may respond rapidly to a higher or lower dose and may find much weaker maintenance doses adequate. For other patients, it may be necessary to have long-term treatments at the rate of 1 to 4 doses per day, in accordance with the physiological requirements of each particular patient. It goes without saying that, for other patients, it will be necessary to prescribe not more than one or two doses per day.

The formulations can be prepared in unit dosage form by any of the methods well known in the art of pharmacy. Such methods include the step of bringing into association the active ingredient with the carrier that constitutes one or more accessory ingredients. In general the formulations are prepared by uniformly and intimately bringing into association the active ingredient with liquid carriers or finely divided solid carriers or both, and then, if necessary, shaping the product.

The formulations may be presented in unit-dose or multi-dose containers, for example sealed ampoules and vials with elastomeric stoppers, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

Compounds of the invention may be prepared by the application or adaptation of known methods, by which is meant methods used heretofore or described in the literature, for example those described by R. C. Larock in Comprehensive Organic Transformations, VCH publishers, 1989.

In the reactions described hereinafter it may be necessary to protect reactive functional groups, for example hydroxy, amino, imino, thio or carboxy groups, where these are desired in the final product, to avoid their unwanted participation in the reactions. Conventional protecting groups may be used in accordance with standard practice, for examples see T. W. Greene and P. G. M. Wuts, Protecting Groups in Organic Synthesis, 3rd edition, John Wiley & Sons, Inc., 1999. Suitable amine protecting groups include sulfonyl (e.g., tosyl), acyl (e.g., benzyloxycarbonyl or t-butoxycarbonyl) and arylalkyl (e.g., benzyl), which may be removed by hydrolysis or hydrogenolysis as appropriate. Other suitable amine protecting groups include trifluoroacetyl [—C(═O)CF₃] which may be removed by base catalyzed hydrolysis, or a solid phase resin bound benzyl group, such as a Merrifield resin bound 2,6-dimethoxybenzyl group (Ellman linker) or a 2,6-dimethoxy-4-[2-(polystyrylmethoxy)ethoxy]benzyl, which may be removed by acid catalyzed hydrolysis, for example with TFA.

A compound of Formula (XVI), wherein R¹, R², R³, R⁴, R⁵, R⁶, R⁷ and n are as defined herein, may be prepared by a Suzuki coupling reaction of a corresponding compound of Formula (X), wherein X¹ is bromo or chloro, particularly bromo, with a corresponding boronic acid of Formula (XVII) to provide a corresponding compound of Formula (XVI).

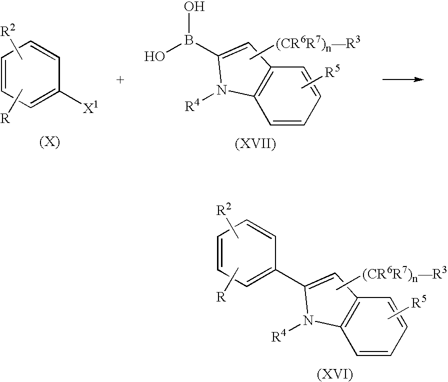

The Suzuki coupling reaction, may conveniently be carried out for example in the presence of PdCl₂(dppf)₂, and CsF, in an inert solvent, such as a mixture of dioxane and water (10:1), at a temperature about 80° C.

A compound of Formula (I), wherein R¹, R², R³, R⁴, R⁵, R⁶ and R⁷ are as defined herein, may be prepared by Fischer indole reaction of a corresponding compound of Formula (III) coupled with a corresponding compound of Formula (IV):

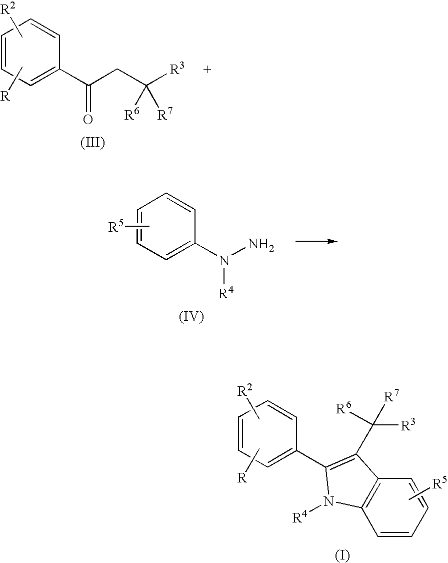

The coupling reaction may conveniently be carried out for example in the presence of p-toluene sulfonic acid and zinc chloride, in an inert solvent, such as glacial acetic acid, in a microwave oven at about 150° C. to about 180° C. The coupling reaction may also conveniently be carried out for example in the presence of potassium hydroxide or sodium hydroxide, in an inert solvent, such as water and glacial acetic acid, at a temperature at about 100° C. The coupling reaction may also conveniently be carried out for example by treating the compound of Formula (III) with HMBA-AM resin from Nova Biochem in the presence of N-hydroxybenzotriazole monohydrate. 1,3-diisopropylcarbodiimide and 4-dimethylaminopyridine, in an inert solvent, such as DCM and DMF, at about room temperature, followed by treating the loaded HMBA-AM resin with the compound of Formula (IV) in the presence of zinc chloride, in an inert solvent, such as glacial acetic acid, at a temperature about 80° C.

A compound of Formula (II), wherein R¹, R², R³, R⁴, R⁵, R⁶ and R⁷ are as defined herein, may be prepared as shown in scheme I, by (1) reacting a corresponding compound of Formula (V) with nitric acid to provide a corresponding compound of Formula (VI), (2) reducing the compound of Formula (VI) to provide a corresponding compound of Formula (VII), (3) converting the compound of Formula (VII) to a corresponding compound of Formula (VIII) by Meerwein reaction, (4) reacting the compound of Formula (VIII) with R¹H (wherein R¹ is —NR′R″) or R¹MgX (wherein R¹ is alkyl, aryl or arylalkyl, and X is halo, particularly chloro or bromo) to provide a corresponding compound of Formula (IX), and (5) coupling the compound of Formula (IX) with a corresponding compound of Formula (IV).

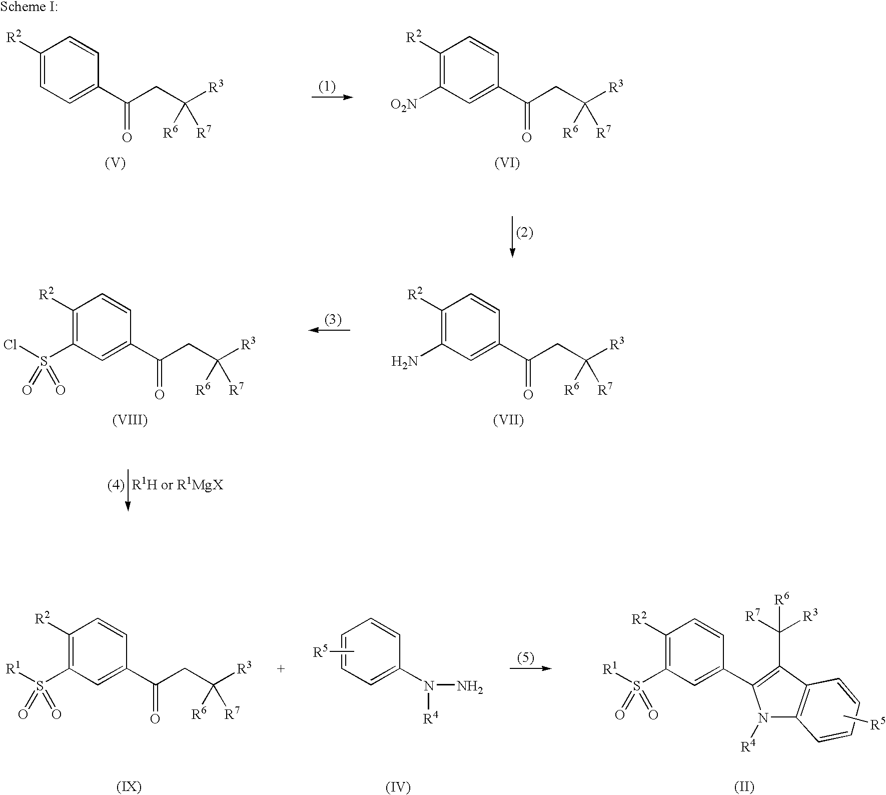

The first step reaction may conveniently be carried out for example at a temperature about −7° C. to 0° C. The second step reaction may conveniently be carried out for example in the presence of sodium bisulfite and hydrochloric acid, in an inert solvent, such as water, at a temperature about 100° C.-105° C. The third step reaction conveniently be carried out for example by first reacting the compound of formula (IX) with sodium nitrite or potassium nitrite in the presence of hydrochloride in an inert solvent, such as THF or DMF, at a temperature about −10° C.-0° C., and then adding Copper (II) chloride and glacial acetic acid saturated with sulfur dioxide to the reaction mixture at a temperature about 0° C. to room temperature. The fourth step reaction may conveniently be carried out for example in an inert solvent, such as THF and ether (when R¹MgX is used), or MeOH and DCM (when R¹H is used), at a temperature about 0° C. to room temperature. The fifth step reaction may be conveniently carried out under the conditions as described above for preparing a compound of Formula (I).

A compound of Formula (I), wherein R, R² and R⁵ are as defined herein, R³ is carboxy, and R⁴, R⁶ and R⁷ are all hydrogen, may be prepared as shown in Scheme II, by (1) a Suzuki coupling reaction of a corresponding compound of Formula (X), wherein X¹ is bromo or chloro, particularly bromo, with a corresponding boronic acid of Formula (XI) to provide a corresponding compound of Formula (XII), (2) deprotecting the compound of Formula (XII) to provide a corresponding compound of Formula (XIII), (3) reacting the compound of Formula (XIII), first with oxalyl chloride, then with MeOH to provide a corresponding compound of Formula (XIV), (4) reducing the compound of Formula (XIV) to provide a corresponding compound of Formula (XV), and (5) hydrolyzing the compound of Formula (XV) to provide a compound of Formula (I) wherein R³ is —COOH. 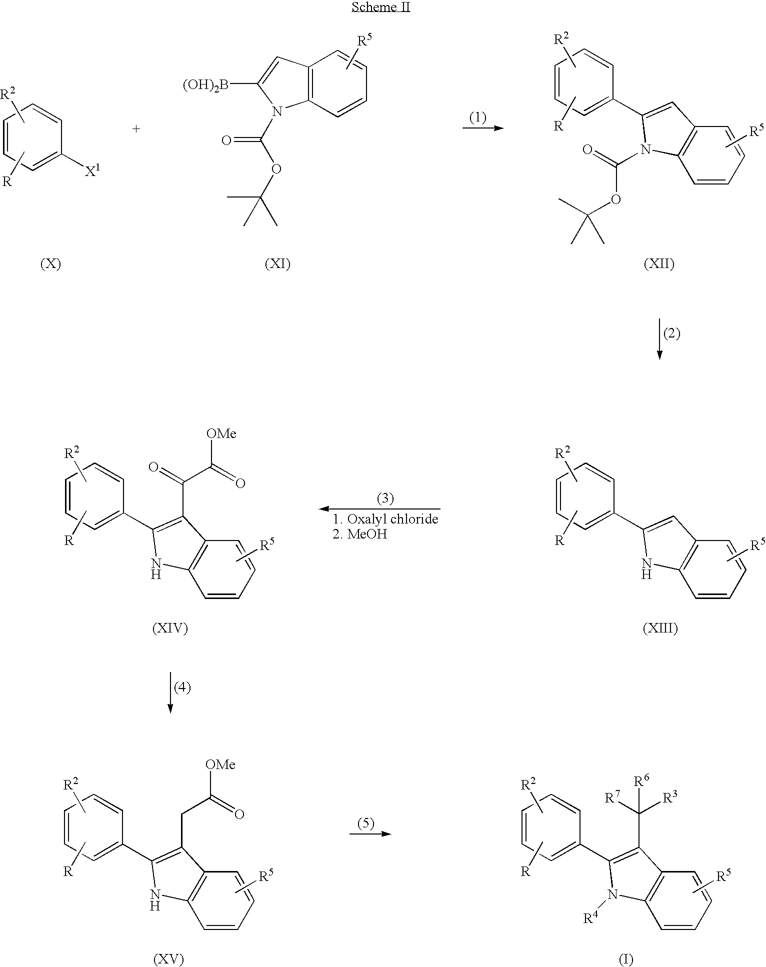
wherein R³ is —COOH, and R⁴, R⁶ and R⁷ is hydrogen

The first step, a Suzuki coupling reaction, may conveniently be carried out for example in the presence of PdCl₂(dppf)₂, and CsF, in an inert solvent, such as a mixture of dioxane and water (10:1), at a temperature about 80° C. The second step of deprotection may conveniently be carried out for example by treating the compound of Formula (XII) with TFA, in an inert solvent, such as DCM, at room temperature. The third step reaction may conveniently be carried out for example, in an inert solvent, such as DCM, at room temperature. The fourth step reduction may conveniently be carried out for example, by reacting the compound of Formula (XIV) with triethylsilane in TFA. The fifth step hydrolysis may conveniently be carried out for example, by alkaline hydrolysis using a base, such as an alkali metal hydroxide, e.g. lithium hydroxide, or an alkali metal carbonate, e.g. potassium carbonate, in the presence of an aqueous/organic solvent mixture, using organic solvents such as dioxane, THF or MeOH, at a temperature from about ambient to about reflux. The hydrolysis of the esters may also be carried out by acid hydrolysis using an inorganic acid, such as hydrochloric acid, in the presence of an aqueous/inert organic solvent mixture, using organic solvents such as dioxane or THF, at a temperature from about 50° C. to about 80° C.

Compounds of the invention may also be prepared by interconversion of other compounds of the invention.

Thus, for example, compounds of Formula (I) wherein R³ is —C(O)—NY¹Y² may be prepared by coupling compounds of Formula (I), in which R³ is carboxy, with an amine of Formula NHY¹Y², to give an amide bond using standard peptide coupling procedures. Examples include (i) coupling in the presence of HBTU and DIEA in DCM at room temperature.

As another example of the interconversion process, an ester prodrugs of the compounds of Formula (XVI) may be prepared by coupling compounds of Formula (XVI), in which R³ is carboxy, with an alcohol of Formula Y³OH (wherein Y³ is alkyl or alkyl substituted by amino, alkylamino or dialkylamino), to give an ester bond using standard coupling procedures. Examples include (i) coupling in the presence of HBTU, and optionally in the presence of DIEA, in DCM at room temperature.

As another example of the interconversion process, compounds of Formula (XVI) wherein R³ is —CH₂OH may be prepared by the reduction of corresponding compounds of Formula (XVI) in which R³ is carboxy. The reduction may conveniently be carried out by means of reaction with lithium aluminum hydride, in an inert solvent, such as THF, and at a temperature from about 0° C. to about reflux temperature.

As another example of the interconversion process, compounds of Formula (XVI), wherein R³ is 5-oxo-4,5-dihydro-1,3,4-oxadiazol-2-yl, may be prepared by reaction of the corresponding compounds of Formula (XVI), wherein R³ is carboxy with hydrazine in the presence of HBTU and DIEA, in an inert solvent, such as DCM, and at a temperature at about room temperature followed by treatment of the resulting hydrazone with CDI in the presence of in an inert solvent, such as 1,4-dioxane, and at refluxing temperature.

According to a further feature of the invention, acid addition salts of the compounds of this invention may be prepared by reaction of the free base with the appropriate acid, by the application or adaptation of known methods. For example, the acid addition salts of the compounds of this invention may be prepared either by dissolving the free base in water or aqueous alcohol solution or other suitable solvents containing the appropriate acid and isolating the salt by evaporating the solution, or by reacting the free base and acid in an organic solvent, in which case the salt separates directly or can be obtained by concentration of the solution.

The acid addition salts of the compounds of this invention can be regenerated from the salts by the application or adaptation of known methods. For example, parent compounds of the invention can be regenerated from their acid addition salts by treatment with an alkali, e.g. aqueous sodium bicarbonate solution or aqueous ammonia solution.

Compounds of this invention can be regenerated from their base addition salts by the application or adaptation of known methods. For example, parent compounds of the invention can be regenerated from their base addition salts by treatment with an acid, e.g. hydrochloric acid.

Compounds of the present invention may be conveniently prepared, or formed during the process of the invention, as solvates (e.g. hydrates). Hydrates of compounds of the present invention may be conveniently prepared by recrystallization from an aqueous/organic solvent mixture, using organic solvents such as dioxane, THF or MeOH.

According to a further feature of the invention, base addition salts of the compounds of this invention may be prepared by reaction of the free acid with the appropriate base, by the application or adaptation of known methods. For example, the base addition salts of the compounds of this invention may be prepared either by dissolving the free acid in water or aqueous alcohol solution or other suitable solvents containing the appropriate base and isolating the salt by evaporating the solution, or by reacting the free acid and base in an organic solvent, in which case the salt separates directly or can be obtained by concentration of the solution.

The starting materials and intermediates may be prepared by the methods described in the present application or adaptation of known methods.

The compounds of the invention, their methods or preparation and their biological activity will appear more clearly from the examination of the following examples that are presented as an illustration only and are not to be considered as limiting the invention in its scope. Compounds of the invention are identified, for example, by the following analytical methods.

High Pressure Liquid Chromatography—Mass Spectrometry (LCMS) experiments to determine retention times (R_{T}) and associated mass ions are performed using one of the following methods.

Mass Spectra (MS) are recorded using a Micromass LCT mass spectrometer. The method is positive electrospray ionization, scanning mass m/z from 100 to 1000. Liquid chromatography is performed on a Hewlett Packard 1100 Series Binary Pump & Degasser; stationary phase: Phenomenex Synergi 2μ Hydro-RP 20×4.0 mm column, mobile phase: A=0.1% formic acid (FA) in water, B=0.1% FA in acetonitrile. Injection volume of 5 μL by CTC Analytical PAL System. Flow is 1 mL/minute. Gradient is 10% B to 90% B in 3 minutes and 90% B to 100% B in 2 minutes. Auxiliary detectors are: Hewlett Packard 1100 Series UV detector, wavelength=220 nm and Sedere SEDEX 75 Evaporative Light Scattering (ELS) detector temperature=46° C., N₂ pressure=4 bar.

300 MHz ¹H nuclear magnetic resonance spectra (NMR) are recorded at ambient temperature using a Varian Mercury (300 MHz) spectrometer with an ASW 5 mm probe. In the NMR chemical shifts (δ) are indicated in parts per million (ppm) with reference to tetramethylsilane (TMS) as the internal standard.

As used in the examples and preparations that follow, as well as the rest of the application, the terms used therein shall have the meanings indicated: “kg” refers to kilograms, “g” refers to grams, “mg” refers to milligrams, “μg” refers to micrograms, “mol” refers to moles, “mmol” refers to millimoles, “M” refers to molar, “mM” refers to millimolar, “μM” refers to micromolar, “nM” refers to nanomolar, “L” refers to liters, “mL” or “ml” refers to milliliters, “μL” refers to microliters, “° C.” refers to degrees Celsius, “mp” or “m.p.” refers to melting point, “bp” or “b.p.” refers to boiling point, “mm of Hg” refers to pressure in millimeters of mercury, “cm” refers to centimeters, “nm” refers to nanometers, “abs.” refers to absolute, “conc.” refers to concentrated, “c” refers to concentration in g/mL, “rt” refers to room temperature, “TLC” refers to thin layer chromatography, “HPLC” refers to high performance liquid chromatography, “i.p.” refers to intraperitoneally, “i.v.” refers to intravenously, “s”=singlet, “d”=doublet; “t”=triplet; “q”=quartet; “m”=multiplet, “dd”=doublet of doublets; “br”=broad, “LC”=liquid chromatograph, “MS”=mass spectrograph, “ESI/MS”=electrospray ionization/mass spectrograph, “R_{T}”=retention time, “M”=molecular ion, “PSI”=pounds per square inch, “DMSO”=dimethyl sulfoxide, “DMF”=N,N-dimethylformamide, “CDI”=1,1′-carbonyldiimidazole, “DCM” or “CH₂Cl₂”=dichloromethane, “HCl”=hydrochloric acid, “SPA”=Scintillation Proximity Assay, “ATTC”=American Type Culture Collection, “FBS”=Foetal Bovine Serum, “MEM”=Minimal Essential Medium, “CPM”=Counts Per Minute, “EtOAc”=ethyl acetate, “PBS”=Phosphate Buffered Saline, “TMD”=transmembrane domain, “IBMX”=3-isobutyl-1-methylxanthine, “cAMP”=cyclic adenosine monophosphate, “IUPAC”=International Union of Pure and Applied Chemistry, “MHz”=megahertz, “PEG”=polyethylene glycol, “MeOH”=methanol, “N”=normality, “THF”=tetrahydrofuran, “h”=hours, “min”=minute(s), “MeNH₂”=methyl amine, “N₂”=nitrogen gas, “iPrOH”=isopropyl alcohol, “O.D.”=outer diameter, “MeCN” or “CH₃CN”=acetonitrile, “Et₂O”=ethyl ether, “TFA”=trifluoroacetic acid, “Prep LC”=preparatory “flash” liquid chromatography, “SPE”=solid phase extraction, “LAH”=lithium aluminum hydride, “pmol”=picomolar, “heptane”=n-heptane, “HMBA-AM” resin=4-hydroxymethylbenzoic acid amino methyl resin, “PdCl₂(dppf)₂”=1,1′-bis(diphenylphosphino)ferrocene-palladium (II) dichloride DCM complex, “HBTU”=2-(1H-benzotriazol-1-yl)-1,1,3,3-tetramethyluronium hexafluorophosphate, “DIEA”=diisopropylethylamine, “CsF”=cesium fluoride, “MeI”=methyl iodide, “˜”=approximately.

### EXAMPLES

### Example 1

### (a) [2-(4-Chloro-3-cyclohexylsulfamoyl-phenyl)-1H-indol-3-yl]-acetic acid

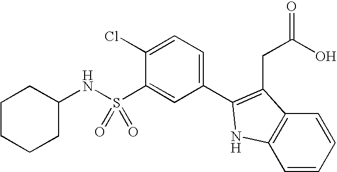 Method A:

Step 1. Fuming nitric acid (1.5 L) is cooled to about −5° C. in an ice/salt bath. Over a period of 30 minutes, 4-(4-chloro-phenyl)-4-oxo-butyric acid (150 g) is added in portions to the mechanically stirred solution, and the reaction mixture is stirred at the temperature between about −5° C. and about −7° C. for 3.5 hours. The reaction mixture is poured onto crushed ice/water (3 L) and stirred overnight at room temperature. The solid material is filtered, washed with water until the washes are neutral, air dried, and finally dried in a vacuum oven at about 85° C. to afford 4-(4-chloro-3-nitro-phenyl)-4-oxo-butyric acid as a solid (159.1 g).

Step 2. To a mechanically stirred suspension of 4-(4-chloro-3-nitro-phenyl)-4-oxo-butyric acid (150 g) in water (900 mL) and concentrated HCl (12 mL) is added sodium bisulfite solution (393 g, in 800 mL of water) over a period of 40 minutes at 100-105° C. After the addition, the mixture is refluxed for 1 hour, and the pH is adjusted to ˜2 by the addition of 4 N HCl (100 mL). The mixture is refluxed for an additional 30 minutes, cooled to room temperature and filtered to afford 4-(3-amino-4-chloro-phenyl)-4-oxo-butyric acid as a solid (79.3 g). LCMS: R_{T}=2.39 minutes, MS: 228 (M+H); ¹H NMR (300 MHz, DMSO-D₆) δ 2.51 (t, J=6 Hz, 2H) 3.11 (t, J=6 Hz, 2H) 5.58 (s, 2H), 7.1 (dd, J=6.2 Hz, J=2 Hz, 1H) 7.29 (d, J=8 Hz, 1H) 7.36 (d, J=2 Hz, 1H) 12.08 (broad s, 1H).

Step 3: 4-(3-Amino-4-chloro-phenyl)-4-oxo-butyric acid (16.2 g) in DMF (20 mL) is added to a mixture of concentrated HCl (35 mL) and ice (150 g). A solution of sodium nitrite (5.25 g) in water (18 mL) is added via pipette below the surface of the solution over 5 minutes at a temperature between −5° C. and −10° C. The reaction mixture is warmed to 0° C. and stirred for 15 min. This solution is slowly added at room temperature to a mixture of copper chloride dihydrate (5.58 g) in glacial acetic acid (175 mL) that has been saturated with sulfur dioxide gas. The resulting solution is stirred 45 minutes at room temperature, water (500 mL) is added and the solution is stirred for 1 hour. The flask is cooled to 10° C. and the solid is filtered and washed with water to afford 4-(4-chloro-3-chlorosulfonyl-phenyl)-4-oxo-butyric acid as a solid [12.94 g, Intermediate (1)]. LCMS: R_{T}=2.68 minutes, MS: 310 (M+H); ¹H NMR (300 MHz, DMSO-D₆) δ ppm 2.56 (t, J=6 Hz, 2H) 3.19 (t, J=6 Hz, 2H) 7.51 (d, J=8 Hz, 1H) 7.87 (dd, J=6 Hz, J=2 Hz, 1H) 8.39 (d, J=2 Hz, 1H) 12.66 (broad s, 1H).

Step 4: 4-(4-Chloro-3-chlorosulfonyl-phenyl)-4-oxo-butyric acid (2 g) is added to a stirred solution of cyclohexylamine (1.56 g) in DCM:MeOH mixture (1:1, 50 mL) at 0° C. The reaction mixture is warmed to room temperature and stirred for 20 hours. The reaction mixture is acidified with 2 N aqueous HCl (pH˜2) and extracted twice with methylene chloride. The combined organic layer is washed with water, dried over sodium sulfate and evaporated in vacuo to afford 4-(4-chloro-3-cyclohexylsulfamoyl-phenyl)-4-oxo-butyric acid as light brown viscous oil (1.7 g). LCMS: R_{T}=2.9 minutes, MS: 374 (M+H); ¹H NMR (300 MHz, DMSO-D₆) δ 0.8-1.8 (m, 10H) 2.61 (t, J=6 Hz, 2H) 3.04 (m, 1H) 3.3 (m, 2H) 7.8 (d, J=8 Hz, 1H) 8.06 (d, J=8 Hz, 1H) 8.2 (d, J=8 Hz, 1H) 8.46 (s, 1H) 12.2 (broad s, 1H).

Step 5: To a mixture of 4-(4-chloro-3-cyclohexylsulfamoyl-phenyl)-4-oxo-butyric acid (0.56 g), zinc chloride (205 mg), p-toluene sulfonic acid monohydrate (285 mg) in glacial acetic acid (8 mL) in a microwave vessel is added phenyl hydrazine (165 mg). The capped vessel is heated in a microwave at 180° C. for 40 minutes. The reaction mixture is diluted with EtOAc, transferred to a conical flask, and aqueous 2 N HCl (˜50 mL) is added. The organic layer is separated and the aqueous layer is extracted with EtOAc. The combined organic layer is washed with water, dried over sodium sulfate and concentrated. The residue is purified by flash column chromatography on silica gel eluting with 30% to 100% EtOAc in heptane. The obtained product is rechromatographed on silica gel column eluting with 0% to 30% MeOH in DCM to afford [2-(4-chloro-3-cyclohexylsulfamoyl-phenyl)-1H-indol-3-yl]-acetic acid as a solid (81 mg). LCMS: R_{T}=3.05 minutes, MS: 447 (M+H); ¹H NMR (300 MHz, DMSO-D₆) δ 0.8-1.7 (m, 10H) 3.05 (m, 1H) 3.74 (s, 2H) 7.05 (t, J=7 Hz, 1H) 7.15 (t, J=7.0 Hz, 1H) 7.41 (d, J=8.2 Hz, 1H) 7.56 (d, J=8 Hz, 1H) 7.8 (d, J=8.3 Hz, 1H) 7.9 (m, 2H) 8.27 (d, J=2 Hz, 1H) 11.56 (s, 1H) 12.39 (broad s, 1H). IC₅₀=2.2 nM

Method B:

Step 1: A mixture of 4-(4-chloro-3-cyclohexylsulfamoyl-phenyl)-4-oxo-butyric acid [2 g, Intermediate (1)], HBTU (2.5 g), and DIEA (1.4 g) in DCM (50 mL) is stirred at room temperature for 16 hours, and anhydrous MeOH (2 mL) is added. The mixture is stirred at room temperature for 24 hours, and diluted with DCM (˜100 mL). The solution is washed with aqueous 2 HCl, water, dried over sodium sulfate, and concentrated in vacuo. The crude is purified by a short silica gel column chromatography eluting with 10% to 40% EtOAc in heptane to afford 4-(4-chloro-3-cyclohexylsulfamoyl-phenyl)-4-oxo-butyric acid methyl ester (1 g) as an oil. MS: 388 (M+H).

Step 2: A mixture of 4-(4-chloro-3-cyclohexylsulfamoyl-phenyl)-4-oxo-butyric acid methyl ester (500 mg), phenylhydrazine hydrochloride (225 mg), p-toluene sulfonic acid monohydrate (250 mg) in glacial acetic acid (3 mL) in a capped microwave vessel is heated in a microwave at 150=C for 20 minutes. Zinc chloride (180 mg) is added and the resulting mixture is heated in microwave at 160° C. for 20 minutes. The reaction mixture is diluted with EtOAc, transferred to a conical flask and aqueous 2 N HCl (˜50 mL) is added. The organic layer is separated. The aqueous layer is extracted with EtOAc. The combined organic layer is washed with water, dried over sodium sulfate and evaporated in vacuo. The residue is purified by a combination of repeated flash column chromatography on silica gel eluting with 30-70% EtOAc in heptane and preparative HPLC separation (mobile phase acetonitrile-water with 0.1% TFA; gradient 10-100% over 10 minutes) to afford [2-(4-chloro-3-cyclohexylsulfamoyl-phenyl)-1H-indol-3-yl]-acetic acid (110 mg).

Method C:

Step 1: HMBA-AM resin from Nova Biochem (5 g, 1 mmol/g) is swelled in a 9:1 mixture of anhydrous DCM-DMF (75 mL) for 10 minutes. A solution of 4-(4-chloro-3-cyclohexylsulfamoyl-phenyl)-4-oxo-butyric acid (5.6 g) in 9:1 mixture of anhydrous DCM-DMF (25 mL) is added followed by the addition of N-hydroxybenzotriazole monohydrate (2.6 g), 1,3-diisopropylcarbodiimide (1.9 g) and 4-dimethylaminopyridine (0.2 g). The mixture is shaken for 20 hours at room temperature. The resin is filtered and washed successively three times each with DMF, 3:1 DMF-water, THF, DCM, MeOH and Et₂O. The resin is dried in vacuo for 20 hours.

Step 2: 4-(4-Chloro-3-cyclohexylsulfamoyl-phenyl)-4-oxo-butyric acid loaded HMBA-AM resin from Step 1 (3 g) is swelled in glacial acetic acid (60 mL) for 10 minutes followed by the addition of phenylhydrazine hydrochloride (1.5 g) and zinc chloride (1.4 g). The mixture is shaken for 20 hours at 80° C. The resin is filtered and washed successively three times each with DMF, 3:1 DMF-water, THF, MeOH and DCM. The resin is dried in vacuo for 1 hour, and treated with 0.5 M solution of sodium methoxide in MeOH (12 mL) for 1 hour. Water (6 mL) is added, and the mixture is agitated for 30 minutes. The mixture is drained, and the resin is washed with MeOH. The combined filtrates are acidified with 2 N aqueous HCl (pH˜2) and extracted with EtOAc. The organic layer is dried over sodium sulfate and concentrated in vacuo. The residue is purified by silica gel flash column chromatography eluting with 30% to 100% EtOAc in heptane to afford [2-(4-chloro-3-cyclohexylsulfamoyl-phenyl)-1H-indol-3-yl]-acetic acid as a solid (50 mg).

### (b) {2-[3-(Bicyclo[2.2.1]hept-2-ylsulfamoyl)-4-chloro-phenyl]-1H-indol-3-yl}-acetic acid

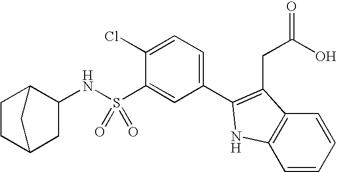

Step 1: By proceeding in a similar manner to Example 1 (a) method A, step 4, but substituting (+/−) endo-2-norbornylamine hydrochloride (1.4 g) and DIEA (3.4 mL) for cyclohexylamine, there is prepared 4-[3-(bicyclo[2.2.1]hept-2-ylsulfamoyl)-4-chloro-phenyl]-4-oxo-butyric acid as a solid (1.9 g). LCMS: R_{T}=2.42 minutes, MS: 386 (M+H); ¹H NMR (300 MHz, DMSO-D₆) δ 0.8-2.1 (m, 10H) 2.61 (t, J=6 Hz, 2H) 3.29 (t, J=6 Hz, 2H) 3.06 (m, 1H) 7.82 (d, J=8 Hz, 1H) 8.18 (m, 2H) 8.43 (d, J=2 Hz, 1H) 12.2 (broad s, 1H).

Step 2: By proceeding in a similar manner to Example 1 (a) method A, step 5, but substituting 4-[3-(bicyclo[2.2.1]hept-2-ylsulfamoyl)-4-chloro-phenyl]-4-oxo-butyric acid (0.58 g) for 4-(4-chloro-3-cyclohexylsulfamoyl-phenyl)-4-oxo-butyric acid, there is prepared {2-[3-(bicyclo[2.2.1]hept-2-ylsulfamoyl)-4-chloro-phenyl]-1H-indol-3-yl}-acetic acid (93 mg). LCMS: R_{T}=3.12 minutes, MS: 459 (M+H); ¹H NMR (300 MHz, CD₃OD) δ 0.8-2.2 (m, 10H) 3.6 (m, 1H) 3.84 (s, 2H) 7.1 (t, J=7.50 Hz, 1H) 7.2 (t, J=7.5 Hz, 1H) 7.42 (d, J=8 Hz, 1H) 7.61 (d, J=7.8 Hz, 1H) 7.71 (d, J=8.2 Hz, 1H) 7.90 (dd, J=6.0, 2.3 Hz, 1H) 8.39 (d, J=2 Hz, 1H). IC₅₀=3 nM

### (c) [2-(4-Chloro-3-hexylsulfamoyl-phenyl)-1H-indol-3-yl]-acetic acid

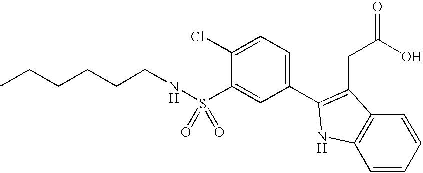

Step 1: By proceeding in a similar manner to Example 1 (a) method A, step 4, but substituting N-hexylamine (1.62 g) for cyclohexylamine, there is prepared 4-(4-chloro-3-hexylsulfamoyl-phenyl)-4-oxo-butyric acid (1.8 g). LCMS: R_{T}=2.57 minutes, MS: 376 (M+H); ¹H NMR (300 MHz, DMSO-D₆) δ 0.8 (t, J=7 Hz, 3H) 1-1.4 (m, 8H) 2.61 (t, J=6 Hz, 2H) 2.85 (m, 2H) 3.29 (t, J=6 Hz, 2H) 7.82 (d, J=8 Hz, 1H) 8.04 (t, J=5 Hz, 1H) 8.2 (dd, J=6 Hz, J=2 Hz, 1H) 8.42 (d, J=2 Hz, 1H) 12.2 (broad s, 1H)

Step 2: By proceeding in a similar manner to Example 1(a) method A, step 5 but substituting 4-(4-chloro-3-hexylsulfamoyl-phenyl)-4-oxo-butyric acid (0.56 g) for 4-(4-chloro-3-cyclohexylsulfamoyl-phenyl)-4-oxo-butyric acid, there is prepared [2-(4-chloro-3-hexylsulfamoyl-phenyl)-1H-indol-3-yl]-acetic acid (54 mg). LCMS: R_{T}=3.21 minutes, MS: 449 (M+H); ¹H NMR (300 MHz, CD₃OD) δ 0.87 (t, J=7 Hz, 3H) 1.3 (m, 6H) 1.48 (m, 2H) 3 (t, J=7 Hz, 2H) 3.82 (s, 2H) 7.06 (t, J=7 Hz, 1H) 7.17 (t, J=7.5 Hz, 1H) 7.42 (d, J=8 Hz, 1H) 7.62 (d, J=8 Hz, 1H) 7.72 (d, J=8 Hz, 1H) 7.93 (dd, J=6, 2 Hz, 1H) 8.39 (d, J=2.3 Hz, 1H). IC₅₀=31 nM

### (d) {2-[4-Chloro-3-(indan-2-ylsulfamoyl)-phenyl]-1H-indol-3-yl}-acetic acid

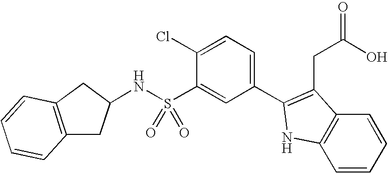

Step 1: By proceeding in a similar manner to Example 1 (a) method A, step 4, but substituting 2-aminoindan (2.14 g) for cyclohexylamine, there is prepared 4-[4-chloro-3-(indan-2-ylsulfamoyl)-phenyl]-4-oxo-butyric acid (2.1 g). LCMS: R_{T}=2.45 minutes, MS: 408 (M+H); ¹H NMR (300 MHz, DMSO-D₆) δ 2.6 (t, J=6 Hz, 2H) 2.84 (m, 2H) 2.95 (m, 2H) 3.3 (m, 2H) 4.03 (m, 1H) 7.1 (m, 4H) 7.86 (d, J=8 Hz, 1H) 8.22 (dd, J=6 Hz, J=2 Hz, 1H) 8.5 (m, 2H) 12.2 (broad s, 1H).

Step 2: By proceeding in a similar manner to Example 1(a) method A, step 5, but substituting 4-[4-chloro-3-(indan-2-ylsulfamoyl)-phenyl]-4-oxo-butyric acid (0.61 g) for 4-(4-chloro-3-cyclohexylsulfamoyl-phenyl)-4-oxo-butyric acid, there is prepared {2-[4-chloro-3-(indan-2-ylsulfamoyl)-phenyl]-1H-indol-3-yl}-acetic acid (66 mg). LCMS: R_{T}=3.11 minutes, MS: 481 (M+H); ¹H NMR (300 MHz, CD₃OD) δ 2.88 (m, 2H), 3.05 (m, 2H) 3.86 (s, 2H) 4.15 (m, 1H) 7-7.22 (m, 6H) 7.41 (d, J=8 Hz, 1H) 7.62 (d, J=8 Hz, 1H) 7.75 (d, J=8 Hz, 1H) 8 (d, J=2 Hz, 1H) 8.44 (d, J=2 Hz, 1H). IC₅₀=9.6 nM

### (e) [2-(4-Chloro-3-cyclopentylsulfamoyl-phenyl)-1H-indol-3-yl]-acetic acid

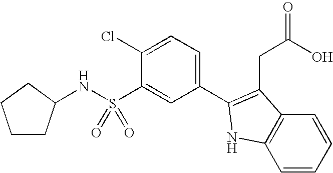

Step 1: By proceeding in a similar manner to Example 1 (a) method A, step 4, but substituting cyclopentylamine (1.37 g) for cyclohexylamine, there is prepared 4-(4-chloro-3-cyclopentylsulfamoyl-phenyl)-4-oxo-butyric acid (1.6 g). LCMS: R_{T}=2.25 minutes, MS: 360 (M+H); ¹H NMR (300 MHz, DMSO-D₆) δ 1.2-1.7 (m, 8H) 2.6 (t, J=6 Hz, 2H) 3.29 (t, J=6 Hz, 2H) 3.5 (m, 1H) 7.82 (d, J=8 Hz, 1H) 8.08 (d, J=8 Hz, 1H) 8.2 (dd, J=6 Hz, J=2 Hz, 1H) 8.46 (d, J=2 Hz, 1H) 12.2 (broad s, 1H).

Step 2: By proceeding in a similar manner to Example 2(a) method A, step 5, but substituting 4-(4-chloro-3-cyclopentylsulfamoyl-phenyl)-4-oxo-butyric acid (0.55 g) for 4-(4-chloro-3-cyclohexylsulfamoyl-phenyl)-4-oxo-butyric acid, there is prepared {2-[4-chloro-3-(cyclopentylsulfamoyl)-phenyl]-1H-indol-3-yl}-acetic acid (48 mg). LCMS: R_{T}=2.96 minutes, MS: 433 (M+H); ¹H NMR (300 MHz, CD₃OD) δ 0.8-1.8 (m, 8H) 3.7 (m, 1H) 3.85 (s, 2H) 7.09 (t, J=8 Hz, 1H) 7.19 (t, J=7 Hz, 1H) 7.42 (d, J=8 Hz, 1H) 7.62 (d, J=8 Hz, 1H) 7.73 (d, J=8 Hz, 1H) 7.93 (dd, J=6 Hz, J=2 Hz, 1H) 8.42 (d, J=2 Hz, 1H).

### (f) {2-[4-Chloro-3-(2,2-dimethyl-propylsulfamoyl)-phenyl]-1H-indol-3-yl}-acetic acid

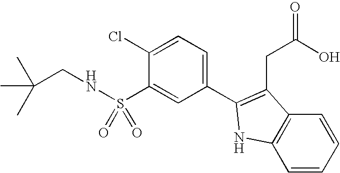

Step 1: By proceeding in a similar manner to Example 1 (a) method A, step 4, but substituting neopentylamine (1.4 g) for cyclohexylamine, there is prepared 4-[4-chloro-3-(2,2-dimethyl-propylsulfamoyl)-phenyl]-4-oxo-butyric acid (1.8 g). LCMS: R_{T}=2.37 minutes, MS: 362 (M+H); ¹H NMR (300 MHz, DMSO-D₆) δ 0.82 (m, 9H) 2.61 (t, J=6 Hz, 2H) 2.69 (d, J=8 Hz, 2H) 3.29 (t, J=6 Hz, 2H) 7.82 (d, J=8 Hz, 1H) 8 (t, J=6 Hz, 1H) 8.2 (dd, J=6 Hz, J=2 Hz, 1H) 8.41 (d, J=2 Hz, 1H) 12.2 (broad s, 1H).

Step 2: By proceeding in a similar manner to Example 2(a) method A, step 5, but substituting 4-[4-chloro-3-(2,2-dimethyl-propylsulfamoyl)-phenyl]-4-oxo-butyric acid (0.55 g) for 4-(4-chloro-3-cyclohexylsulfamoyl-phenyl)-4-oxo-butyric acid, there is prepared {2-[4-chloro-3-(2,2-dimethyl-propylsulfamoyl)-phenyl]-1H-indol-3-yl}-acetic acid (15 mg). LCMS: R_{T}=3.06 minutes, MS: 435 (M+H).

### (g) [2-(4-Chloro-3-isopropylsulfamoyl-phenyl)-1H-indol-3-yl]-acetic acid

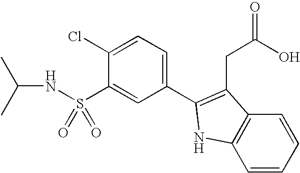

Step 1: By proceeding in a similar manner to Example 1 (a) method A, step 4, but substituting isopropylamine (0.95 g) for cyclohexylamine, there is prepared 4-(4-chloro-3-isopropylsulfamoyl-phenyl)-4-oxo-butyric acid (1.4 g). LCMS: R_{T}=2.02 minutes, MS: 334 (M+H); ¹H NMR (300 MHz, DMSO-D₆) δ 1.01 (d, J=6.4 Hz, 6H) 2.61 (t, J=6 Hz, 2H) 3.29 (t, J=6 Hz, 2H) 3.36 (m, 1H) 7.83 (d, J=8 Hz, 1H) 8.01 (d, J=8 Hz, 1H) 8.2 (dd, J=6 Hz, J=2 Hz, 1H) 8.46 (d, J=2 Hz, 1H) 12.2 (broad s, 1H).

Step 2: By proceeding in a similar manner to Example 1 (a) method A, step 5, but substituting 4-(4-chloro-3-isopropylsulfamoyl-phenyl)-4-oxo-butyric acid (0.5 g) for 4-(4-chloro-3-cyclohexylsulfamoyl-phenyl)-4-oxo-butyric acid, there is prepared [2-(4-chloro-3-isopropylsulfamoyl-phenyl)-1H-indol-3-yl]-acetic acid (64 mg). LCMS: R-T-=2.81 minutes, MS: 407 (M+H); ¹H NMR (300 MHz, CD₃OD) δ 1.12 (d, J=6.5 Hz, 6H) 3.53 (m, 1H) 3.85 (s, 2H) 7.09 (t, J=7.3 Hz, 1H) 7.19 (t, J=7.4 Hz, 1H) 7.43 (d, J=8 Hz, 1H) 7.62 (d, J=8 Hz, 1H) 7.72 (d, J=8.2 Hz, 1H) 7.95 (dd, J=6.2 Hz, J=2 Hz, 1H) 8.41 (d, J=2.3 Hz, 1H). IC₅₀=49 nM

### (h) {2-[4-Chloro-3-(2-cyclohexyl-ethylsulfamoyl)-phenyl]-1H-indol-3-yl}-acetic acid

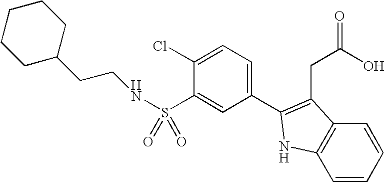

Step 1: By proceeding in a similar manner to Example 1 (a) method A, step 4, but substituting 2-cyclohexyl-ethylamine hydrochloride (0.79 g) and DIEA (1.7 mL) for cyclohexylamine, and using 4-(4-chloro-3-chlorosulfonyl-phenyl)-4-oxo-butyric acid [1 g, Intermediate (1)], there is prepared 4-[4-chloro-3-(2-cyclohexyl-ethylsulfamoyl)-phenyl]-4-oxo-butyric acid (1.1 g). LCMS: R_{T}=2.70 minutes, MS: 402 (M+H); ¹H NMR (300 MHz, DMSO-D₆) δ 0.6-1.8 (m, 13H) 2.5-3.7 (series of m, 6H) 7.85 (d, J=8 Hz, 1H) 8.05 (d, J=6 Hz, 1H) 8.2 (d, J=7 Hz, 1H) 8.42 (d, J=2 Hz, 1H) 12.2 (broad s, 1H).

Step 2: By proceeding in a similar manner to Example 1(a) method A, step 5, but substituting 4-[4-chloro-3-(2-cyclohexyl-ethylsulfamoyl)-phenyl]-4-oxo-butyric acid (0.6 g) for 4-(4-chloro-3-cyclohexylsulfamoyl-phenyl)-4-oxo-butyric acid, there is prepared {2-[4-chloro-3-(2-cyclohexyl-ethylsulfamoyl)-phenyl]-1H-indol-3-yl}-acetic acid (74 mg). LCMS: R_{T}=3.31 minutes, MS: 475 (M+H); ¹H NMR (300 MHz, CD₃OD) δ 0.7-1.8 (m, 13H) 3.04 (m, 2H) 3.87 (s, 2H) 7.08 (t, J=7.2 Hz, 1H) 7.17 (t, J=7.5 Hz, 1H) 7.42 (d, J=8 Hz, 1H) 7.62 (d, J=8 Hz, 1H) 7.73 (d, J=8.2 Hz, 1H) 7.92 (dd, J=6.3 Hz, J=2 Hz, 1H) 8.40 (d, J=2 Hz, 1H). IC₅₀=35 nM

### (i) [2-(4-Chloro-3-phenylsulfamoyl-phenyl) 1H-indol-3-yl]-acetic acid

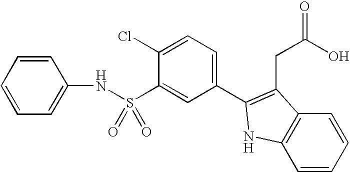

Step 1: By proceeding in a similar manner to Example 1 (a) method A, step 4, but substituting aniline (1.5 g) for cyclohexylamine, there is prepared 4-(4-chloro-3-phenylsulfamoyl-phenyl)-4-oxo-butyric acid (1.8 g). LCMS: R_{T}=2.2 minutes, MS: 368 (M+H); ¹H NMR (300 MHz, DMSO-D₆) δ 2.59 (t, J=6 Hz, 2H) 3.25 (t, J=6 Hz, 2H) 7.01 (t, J=7.4 Hz, 1H) 7.11 (d, J=8.5 Hz, 2H) 7.22 (t, J=8 Hz, 2H) 7.8 (d, J=8 Hz, 1H) 8.18 (dd, J=6 Hz, J=2 Hz I H), 8.47 (d, J=2 Hz, 1H) 10.75 (s, 1H).

Step 2: By proceeding in a similar manner to Example 1 (a) method A, step 5, but substituting 4-(4-chloro-3-phenylsulfamoyl-phenyl)-4-oxo-butyric acid (0.55 g) for 4-(4-chloro-3-cyclohexylsulfamoyl-phenyl)-4-oxo-butyric acid, there is prepared [2-(4-chloro-3-phenylsulfamoyl-phenyl)-1H-indol-3-yl]-acetic acid (155 mg). LCMS: R_{T}=2.9 minutes, MS: 441 (M+H); ¹H NMR (300 MHz, CD₃OD) δ 3.7 (s, 2H) 7.05 (m, 2H) 7.19 (m, 5H) 7.4 (d, J=8.2 Hz, 1H) 7.62 (m, 2H) 7.85 (dd, J=6.2, 2 Hz, 1H) 8.4 (d, J=2 Hz, 1H). IC₅₀=18 mM

### (j) {2-[4-Chloro-3-(cyclohexylmethyl-sulfamoyl)-phenyl]-1H-indol-3-yl}-acetic acid

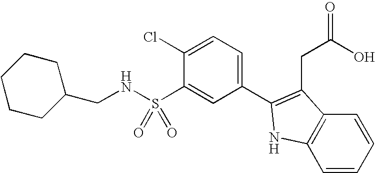

Step 1: By proceeding in a similar manner to Example 2(a) method A, step 4, but substituting aminomethylcyclohexane (1.82 g) for cyclohexylamine, there is prepared 4-[4-chloro-3-(cyclohexylmethyl-sulfamoyl)-phenyl]-4-oxo-butyric acid (1.6 g). MS: 388 (M+H).

Step 2: By proceeding in a similar manner to Example 1 (a) method A, step 5, but substituting 4-[4-chloro-3-(cyclohexylmethyl-sulfamoyl)-phenyl]-4-oxo-butyric acid (0.58 g) for 4-(4-chloro-3-cyclohexylsulfamoyl-phenyl)-4-oxo-butyric acid, there is prepared {2-[4-chloro-3-(cyclohexylmethyl-sulfamoyl)-phenyl]-1H-indol-3-yl}-acetic acid (94 mg). LCMS: R_{T}=3.2 minutes, MS: 461 (M+H); ¹H NMR (300 MHz, CD₃OD) δ 0.8-1.8 (m, 11H) 2.82 (d, J=7 Hz, 2H) 3.85 (s, 2H) 7.09 (t, J=7 Hz, 1H) 7.18 (t, J=7.5 Hz, 1H) 7.42 (d, J=8.3 Hz, 1H) 7.62 (d, J=8 Hz, 1H) 7.72 (d, J=8.2 Hz, 1H) 7.92 (dd, J=6.3, 2 Hz, 1H) 8.37 (d, J=1.7 Hz, 1H).

### (k) {2-[4-Chloro-3-(1-ethyl-propylsulfamoyl)-phenyl]-1H-indol-3-yl}-acetic acid

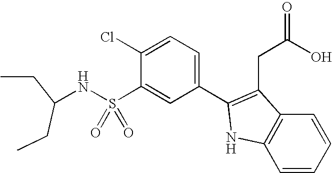

Step 1: By proceeding in a similar manner to Example 1 (a) method A, step 4, but substituting 3-aminopentane (1.4 g) for cyclohexylamine, there is prepared 4-[4-chloro-3-(1-ethyl-propylsulfamoyl)-phenyl]-4-oxo-butyric acid (1.6 g). MS: 362 (M+H).

Step 2: By proceeding in a similar manner to Example 1(a) method A, step 5, but substituting 4-[4-chloro-3-(1-ethyl-propylsulfamoyl)-phenyl]-4-oxo-butyric acid (0.545 g) for 4-(4-chloro-3-cyclohexylsulfamoyl-phenyl)-4-oxo-butyric acid, there is prepared {2-[4-chloro-3-(1-ethyl-propylsulfamoyl)-phenyl]-1H-indol-3-yl}-acetic acid (29 mg). LCMS: R_{T}=3.17 minutes, MS: 435 (M+H); ¹H NMR (300 MHz, DMSO-D₆) δ 0.71 (t, J=7.5 Hz, 6H) 1.34 (m, 4H) 3.02 (m, 1H) 3.6 (s, 2H) 7 (t, J=7.2 Hz, 1H) 7.15 (t, J=7.2 Hz, 1H) 7.39 (d, J=8 Hz, 1H) 7.58 (d, J=8 Hz, 1H) 7.75 (d, J=8.3 Hz, 1H) 7.83 (d, J=8.4 Hz, 1H) 8.03 (d, J=7.2 Hz, 1H) 8.27 (d, J=2 Hz, 1H) 11.48 (s, 1H).

### (l) {2-[4-Chloro-3-(cycloheptylmethyl-sulfamoyl)-phenyl]-1H-indol-3-yl}-acetic acid

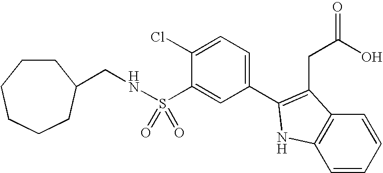

Step 1: By proceeding in a similar manner to Example 1 (a) method A, step 4, but substituting cycloheptylmethyl amine (2 g) for cyclohexylamine, there is prepared 4-[4-chloro-3-(cycloheptyl)methyl-sulfamoyl)-phenyl]-4-oxo-butyric acid (1.9 g). MS: 402 (M+H).

Step 2: By proceeding in a similar manner to Example 1 (a) method A, step 5, but substituting 4-[4-chloro-3-(cycloheptylmethyl-sulfamoyl)-phenyl]-4-oxo-butyric acid (600 mg) for 4-(4-chloro-3-cyclohexylsulfamoyl-phenyl)-4-oxo-butyric acid, there is prepared {2-[4-chloro-3-(cycloheptylmethyl-sulfamoyl)-phenyl]-1H-indol-3-yl}-acetic acid (161 mg). LCMS: R_{T}=3.43 minutes, MS: 475 (M+H); ¹H NMR (300 MHz, CD₃OD) δ 1-1.8 (m, 13H) 2.8 (d, J=7 Hz, 2H) 3.8 (s, 2H) 7.05 (t, J=7 Hz, 1H) 7.15 (t, J=7 Hz, 1H) 7.39 (d, J=8.3 Hz, 1H) 7.58 (d, J=8 Hz, 1H) 7.68 (d, J=8.2 Hz, 1H) 7.88 (dd, J=6.2 Hz, J=2 Hz, 1H) 8.34 (d, J=2.2 Hz, 1H).

### (m) (2-[4-Chloro-3-[(tricyclo[3.3.1.13.7]decan-1-ylmethyl)-sulfamoyl]-phenyl]-1H-indol-3-yl)-acetic acid

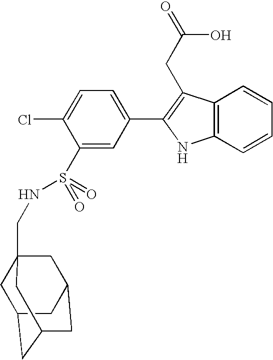

Step 1: By proceeding in a similar manner to Example 1 (a) method A, step 4, but substituting 1-adamantanemethyl amine (1.32 g) for cyclohexylamine, and using 4-(4-chloro-3-chlorosulfonyl-phenyl)-4-oxo-butyric acid [1 g, Intermediate (1)], 1:1 mixture of dichloroethane-ethanol (50 mL) as solvent and the reaction temperature is at 60° C., there is prepared 4-{3-[(adamantan-1-ylmethyl)-sulfamoyl]-4-chloro-phenyl}-4-oxo-butyric acid (0.67 g). MS: 440 (M+H).

Step 2: By proceeding in a similar manner to Example 1(a) method A, step 5, but substituting 4-{3-[(adamantan-1-ylmethyl)-sulfamoyl]-4-chloro-phenyl}-4-oxo-butyric acid (660 mg) for 4-(4-chloro-3-cyclohexylsulfamoyl-phenyl)-4-oxo-butyric acid, there is prepared (2-{4-chloro-3-[(tricyclo[3.3.1.13.7]decan-1-ylmethyl)-sulfamoyl]-phenyl}-1H-indol-3-yl)-acetic acid (68 mg). LCMS: R_{T}=3.64 minutes, MS: 513 (M+H); ¹H NMR (300 MHz, DMSO-D₆) δ 1.2-1.9 (m, 15H) 2.6 (d, J=6.2 Hz, 2H) 3.72 (s, 2H) 7.03 (t, J=8 Hz, 1H) 7.13 (t, J=7 Hz, 1H) 7.4 (d, J=8 Hz, 1H) 7.5 (d, J=7.7 Hz, 1H) 7.8 (m, 2H) 7.9 (d, J=8.2 Hz, 1H) 8.21 (d, J=2 Hz, 1H) 11.5 (s, 1H) 12.4 (broad s, 1H). IC₅₀=8.6 nM

### (n) [2-(4-Chloro-3-cycloheptylsulfamoyl-phenyl)-1H-indol-3-yl]-acetic acid

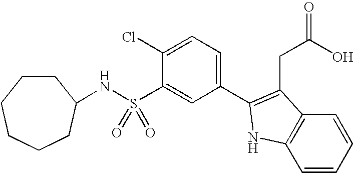

Step 1: By proceeding in a similar manner to Example 1 (a) method A, step 4, but substituting cycloheptylamine (0.45 g) for cyclohexylamine, and using 4-(4-chloro-3-chlorosulfonyl-phenyl)-4-oxo-butyric acid [0.5 g, Intermediate (1)], there is prepared 4-[4-chloro-3-(cycloheptylsulfamoyl)-phenyl]-4-oxo-butyric acid (0.51 g). MS: 388 (M+H).

Step 2: By proceeding in a similar manner to Example 1(a) method A, step 5, but substituting 4-[4-chloro-3-(cycloheptylsulfamoyl)-phenyl]-4-oxo-butyric acid (500 mg) for 4-(4-chloro-3-cyclohexylsulfamoyl-phenyl)-4-oxo-butyric acid, and using zinc chloride (180 mg), p-toluene sulfonic acid monohydrate (250 mg), phenylhydrazine (140 mg) and glacial acetic acid (4 mL), there is prepared [2-(4-chloro-3-cycloheptylsulfamoyl-phenyl)-1H-indol-3-yl]-acetic acid as a solid (94 mg). LCMS: R_{T}=3.27 minutes, MS: 461 (M+H); ¹H NMR (300 MHz, DMSO-D₆) δ 1.1-1.8 (m, 12H) 3.25 (m, 1H) 3.72 (s, 2H) 7.03 (t, J=7 Hz, 1H) 7.15 (t, J=7.0 Hz, 1H) 7.39 (d, J=8 Hz, 1H) 7.54 (d, J=7.8 Hz, 1H) 7.78 (d, J=8.5 Hz, 1H) 7.9 (m, 2H) 8.25 (d, J=2.3 Hz, 1H) 11.54 (s, 1H) 12.38 (broad s, 1H).

### (o) {2-[4-Chloro-3-(tetrahydro-pyran-4-ylsulfamoyl)-phenyl]-1H-indol-3-yl}-acetic acid

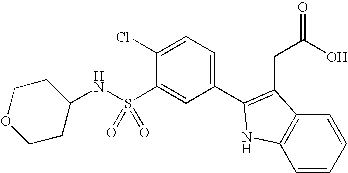

Step 1: By proceeding in a similar manner to Example 1 (a) method A, step 4, but substituting tetrahydro-pyran-4-ylamine (0.4 g) for cyclohexylamine, and using 4-(4-chloro-3-chlorosulfonyl-phenyl)-4-oxo-butyric acid [0.5 g, Intermediate (1)], there is prepared 4-[4-chloro-3-(tetrahydro-pyran-4-ylsulfamoyl)-phenyl]-4-oxo-butyric acid (0.52 g). MS: 376 (M+H).

Step 2: By proceeding in a similar manner to Example 1(a) method A, step 5, but substituting 4-[4-chloro-3-(tetrahydro-pyran-4-ylsulfamoyl)-phenyl]-4-oxo-butyric acid (500 mg) for 4-(4-chloro-3-cyclohexylsulfamoyl-phenyl)-4-oxo-butyric acid, and using zinc chloride (180 mg), p-toluene sulfonic acid monohydrate (250 mg), phenylhydrazine (140 mg) and glacial acetic acid (4 mL), there is prepared 2-[4-chloro-3-(tetrahydro-pyran-4-ylsulfamoyl)-phenyl]-1H-indol-3-yl}-acetic acid as a powder (140 mg). LCMS: R_{T}=2.69 minutes, MS: 449 (M+H); ¹H NMR (300 MHz, DMSO-D₆) δ 1.5 (m, 4H) 3.18 (m, 3H) 3.7 (m, 4H) 7.03 (t, J=7 Hz, 1H) 7.15 (t, J=7 Hz, 1H) 7.39 (d, J=8.3 Hz, 1H) 7.54 (d, J=7.8 Hz, 1H) 7.78 (d, J=8.3 Hz, 1H) 7.9 (dd, J=6.3, 2.3 Hz, 1H) 8.08 (d, J=8 Hz, 1H) 8.26 (d, J=8 Hz, 1H) 11.54 (s, 1H) 12.38 (broad s, 1H). IC₅₀=52 nM (p) {2-[4-Chloro-3-(piperidine-1-sulfonyl)-phenyl]-1H-indol-3-yl}-acetic acid

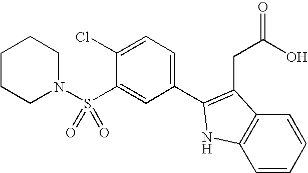

Step 1: By proceeding in a similar manner to Example 1 (a) method A, step 4, but substituting piperidine (2.1 g) for cyclohexylamine, there is prepared 4-[4-chloro-3-(piperidine-1-sulfonyl)-phenyl]-4-oxo-butyric acid as a solid (0.95 g). LCMS: R_{T}=2.73 minutes, MS: 360 (M+H); ¹H NMR (300 MHz, DMSO-D₆) δ 1.5 (m, 6H) 2.6 (t, J=6 Hz, 2H) 3.3 (m, 6H) 7.87 (d, J=8 Hz, 1H) 8.23 (dd, J=6 Hz, J=2 Hz 1H) 8.4 (d, J=2 Hz, 1H) 12.19 (broad s, 1H).

Step 2: By proceeding in a similar manner to Example 1 (a) method A, step 5, but substituting {2-[4-chloro-3-(piperidine-1-sulfonyl)-phenyl]-1H-indol-3-yl}-acetic acid (360 mg) for 4-(4-chloro-3-cyclohexylsulfamoyl-phenyl)-4-oxo-butyric acid, and using zinc chloride (140 mg), p-toluene sulfonic acid monohydrate (190 mg), phenylhydrazine (110 mg) and glacial acetic acid (3 mL), and the reaction temperature is at 160° C., there is prepared {2-[4-chloro-3-(piperidine-1-sulfonyl)-phenyl]-1H-indol-3-yl}-acetic acid (14 mg). LCMS: R_{T}=3.42 minutes, MS: 433 (M+H). IC₅₀=678 nM

### (q) [2-(4-Chloro-3-methylsulfamoyl-phenyl)-1H-indol-3-yl]-acetic acid

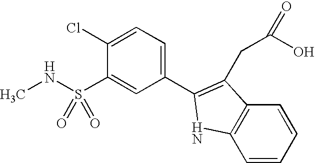

Step 1: By proceeding in a similar manner to Example 1 (a) method A, step 4, but substituting 1 M solution of methylamine in THF (25 mL) for cyclohexylamine, there is prepared 4-(4-chloro-3-methylsulfamoyl-phenyl)-4-oxo-butyric acid (1.4 g). LCMS: R_{T}=2.01 minutes, MS: 306 (M+H); ¹H NMR (300 MHz, DMSO-D₆) δ 2.6 (t, J=6 Hz, 2H) 3.3 (m, 5H) 7.85 (m, 2H) 8.21 (dd, J=6.3 Hz, J=2 Hz, 1H) 8.4 (d, J=2 Hz, 1H) 12.19 (broad s, 1H).

Step 2: By proceeding in a similar manner to Example 1 (a) method A, step 5, but substituting 4-(4-chloro-3-methylsulfamoyl-phenyl)-4-oxo-butyric acid (300 mg) for 4-(4-chloro-3-cyclohexylsulfamoyl-phenyl)-4-oxo-butyric acid, and using zinc chloride (140 mg), p-toluene sulfonic acid monohydrate (190 mg), phenylhydrazine (110 mg) and glacial acetic acid (3 mL), there is prepared [2-(4-chloro-3-methylsulfamoyl-phenyl)-1H-indol-3-yl]-acetic acid (28 mg). LCMS: R_{T}=2.64 minutes, MS: 379 (M+H); ¹H NMR (300 MHz, DMSO-D₆) δ 2.61 (t, J=6 Hz, 2H) 3.28 (t, J=6.5 Hz, 2H) 7.8 (m, 3H) 8.18 (dd, J=6 Hz, J=2 Hz 1H) 8.47 (d, J=2 Hz, 1H) 12.2 (broad s, 1H).

### (r) [2-(4-Chloro-3-sulfamoyl-phenyl)-1H-indol-3-yl]-acetic acid

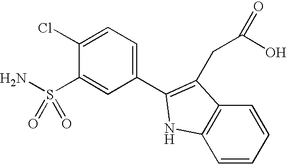

Step 1: 4-(4-Chloro-3-chlorosulfonyl-phenyl)-4-oxo-butyric acid [2 g, Intermediate (1)] is added to a 7N solution of ammonia in MeOH (100 mL) at 0° C. Additional anhydrous MeOH (100 mL) is added and the reaction mixture is stirred at room temperature for 20 hours. The mixture is concentrated in vacuo. The residue is dissolved in EtOAc (˜200 mL), washed with aqueous 2 N HCl (1001 mL) and water, dried over sodium sulfate and concentrated in vacuo to afford 4-(4-chloro-3-sulfamoyl-phenyl)-4-oxo-butyric acid (1.2 g). LCMS: R_{T}=2.48 minutes, MS: 313 (M+Na).

Step 2: By proceeding in a similar manner to Example 2(a) method A, step 5, but substituting 4-(4-chloro-3-sulfamoyl-phenyl)-4-oxo-butyric acid (300 mg) for 4-(4-chloro-3-cyclohexylsulfamoyl-phenyl)-4-oxo-butyric acid, and using zinc chloride (140 mg), p-toluene sulfonic acid monohydrate (190 mg), phenylhydrazine (110 mg) and glacial acetic acid (2 mL), and the reaction temperature is at 160° C., there is prepared [2-(4-chloro-3-sulfamoyl-phenyl)-1H-indol-3-yl]-acetic acid as a solid (8 mg). LCMS: R_{T}=2.47 minutes, MS: 365 (M+H).

### (s) [5-tert-Butyl-2-(4-chloro-3-cyclohexylsulfamoyl-phenyl)-1H-indol-3-yl]-acetic acid

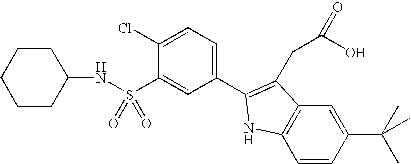

By proceeding in a similar manner to Example 1 (a) method A, step 5, but substituting (4-tert-butyl-phenyl)-hydrazine (0.33 g) for phenylhydrazine, and using 4-(4-chloro-3-cyclohexylsulfamoyl-phenyl)-4-oxo-butyric acid (0.6 g), zinc chloride (0.22 g) and p-toluene sulfonic acid (0.31 g), there is prepared [5-tert-butyl-2-(4-chloro-3-cyclohexylsulfamoyl-phenyl)-1H-indol-3-yl]-acetic acid (107 mg). LCMS: R_{T}=3.6 minutes, MS: 503 (M+H); ¹H NMR (300 MHz, CD₃OD) δ 0.8-1.8 (m, 19H) 3.1 (m, 1H) 3.8 (s, 2H) 7.3 (d, J=8 Hz, 2H) 7.59 (s, 1H) 7.67 d, J=8.3 Hz, 1H) 7.88 (dd, J=6 Hz, J=2.2 Hz, 1H) 8.36 (d, J=2.2 Hz, 1H). IC₅₀=4 nM

### (t) [2-(4-Chloro-3-cyclohexylsulfamoyl-phenyl)-5-methyl-1H-indol-3-yl]-acetic acid

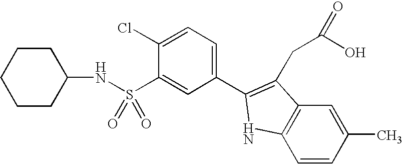

By proceeding in a similar manner to Example 1 (a) method A, step 5, but substituting p-tolyl-hydrazine hydrochloride (0.26 g) for phenylhydrazine, and using 4-(4-chloro-3-cyclohexylsulfamoyl-phenyl)-4-oxo-butyric acid (0.6 g), zinc chloride (0.22 g) and p-toluene sulfonic acid (0.31 g), there is prepared [2-(4-chloro-3-cyclohexylsulfamoyl-phenyl)-5-methyl-1H-indol-3-yl]-acetic acid (36 mg). LCMS: R_{T}=2.82 minutes, MS: 461 (M+H); ¹H NMR (300 MHz, CD₃OD) δ 0.8-1.8 (m, 10H) 2.42 (s, 3H) 3.1 (m, 1H) 3.77 (s, 2H) 6.98 (dd, J=7 Hz, J=1.5 Hz, 1H) 7.27 (d, J=8.5 Hz, 1H) 7.36 (s, 1H) 7.66 (d, J=8.3 Hz, 1H) 7.87 (dd, J=6.3 Hz, J=2.2 Hz, 1H) 8.35 (d, J=2.2 Hz, 1H).

### (u) [2-(4-Chloro-3-cyclohexylsulfamoyl-phenyl)-5-isopropyl-1H-indol-3-yl]-acetic acid

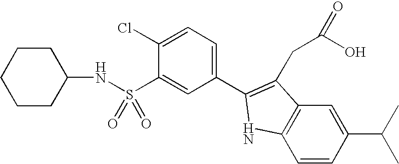

By proceeding in a similar manner to Example 1 (a) method A, step 5, but substituting (4-isopropyl-phenyl)-hydrazine (0.25 g) for phenylhydrazine, and using 4-(4-chloro-3-cyclohexylsulfamoyl-phenyl)-4-oxo-butyric acid (0.6 g), zinc chloride (0.22 g) and p-toluene sulfonic acid (0.31 g), there is prepared [2-(4-chloro-3-cyclohexylsulfamoyl-phenyl)-5-isopropyl-1H-indol-3-yl]-acetic acid (43 mg). LCMS: R_{T}=3.51 minutes, MS: 489 (M+H); ¹H NMR (300 MHz, CD₃OD) δ 0.8-1.8 (m, 16H) 2.97-3.2 (m, 2H) 3.8 (s, 2H) 7.08 (dd, J=7 Hz, J=1.5 Hz, 1H) 7.3 (d, J=8.2 Hz, 1H) 7.42 (s, 1H) 7.66 (d, J=8.2 Hz, 1H) 7.89 (dd, J=6 Hz, J=2.2 Hz, 1H) 8.35 (d, J=2.2 Hz, 1H).

### (v) [2-(4-Chloro-3-cyclohexylsulfamoyl-phenyl)-5-trifluoromethoxy-1H-indol-3-yl]-acetic acid

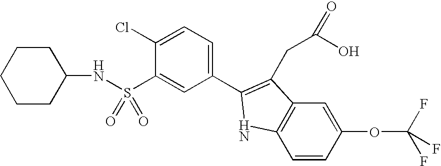

By proceeding in a similar manner to Example 1(a) method A, step 5, but substituting (4-trifluoromethoxy-phenyl)-hydrazine (0.25 g) for phenylhydrazine, and using 4-(4-chloro-3-cyclohexylsulfamoyl-phenyl)-4-oxo-butyric acid (0.5 g), p-toluene sulfonic acid (0.26 g), zinc chloride (0.18 g) and glacial acetic acid (4 mL), there is prepared [2-(4-chloro-3-cyclohexylsulfamoyl-phenyl)-5-trifluoromethoxy-1H-indol-3-yl]-acetic acid as a solid (41 mg). LCMS: R_{T}=3.38 minutes, MS: 531 (M+H); ¹H NMR (300 MHz, DMSO-D₆) δ 0.8-1.7 m, 10H) 3.04 (m, 1H) 3.76 (s, 2H) 7.14 (d, J=7.5 Hz, 1H) 7.49 (d, J=8.7 Hz, 1H) 7.54 (s, 1H) 7.82 (d, J=8.2 Hz, 1H) 7.9 (dd, J=6 Hz, J=2 Hz, 11H) 7.95 (d, J=8 Hz, 1H) 8.26 (d, J=2 Hz, 1H) 11.86 (s, 1H) 12.46 (broad s, 1H). IC₅₀=4.2 nM

### (w) [2-(3-Benzylsulfamoyl-4-chloro-phenyl)-1H-indol-3-yl]-acetic acid

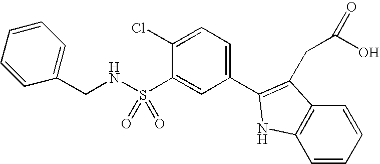

Step 1: By proceeding in a similar manner to Example 1(a) method A, step 4, but substituting benzylamine (1.73 g) for cyclohexylamine, there is prepared 4-(3-benzylsulfamoyl-4-chloro-phenyl)-4-oxo-butyric acid (1.9 g). LCMS: R_{T}=2.14 minutes, MS: 382 (M+H); ¹H NMR (300 MHz, DMSO-D₆) δ 2.6 (t, J=6 Hz, 2H), 3.24 (t, J=6 Hz, 2H), 4.14 (d, J=6 Hz, 2H), 7.15 (m, 5H), 7.71 (d, J=8 Hz, 1H), 8.1 (dd, J=6.0, 2 Hz, 1H), 8.31 (d, J=2 Hz, 1H), 8.62 (t, J=6 Hz, 1H), 12.2 (broad s, 1H).

Step 2: By proceeding in a similar manner to Example 1 (a) method A, step 5, but substituting 4-(3-benzylsulfamoyl-4-chloro-phenyl)-4-oxo-butyric acid (0.5 g) for 4-(4-chloro-3-cyclohexylsulfamoyl-phenyl)-4-oxo-butyric acid and using zinc chloride (180 mg), p-toluene sulfonic acid monohydrate (250 mg), phenylhydrazine (150 mg), glacial acetic acid (8 mL), and purification is by preparative HPLC separation (mobile phase: acetonitrile-water with 0.1% TFA; gradient 10-100% over 10 minutes), there is prepared [2-(3-benzylsulfamoyl-4-chloro-phenyl)-1H-indol-3-yl]-acetic acid (90 mg). LCMS: R_{T}=2.58 minutes, MS: 455 (M+H); ¹H NMR (300 MHz, CD₃OD) δ 3.79 (s, 2H) 4.21 (s, 2H) 7-7.25 (m, 7H) 7.39 (d, J=8 Hz, 1H) 7.58 (apparent m, 2H) 7.8 (dd, J=6 Hz, J=2.2 Hz, 1H) 8.24 (d, J=2.1 Hz, 1H).

### (x) {2-[4-Chloro-3-(cyclohexyl-methyl-sulfamoyl)-phenyl]-1H-indol-3-yl}-acetic acid

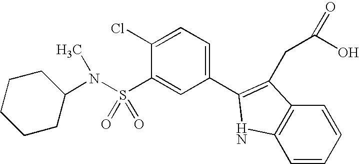

Step 1: By proceeding in a similar manner to Example 1 (a) method A, step 4, but substituting cyclohexyl-methyl-amine (1.8 g) for cyclohexylamine, there is prepared 4-[4-chloro-3-(cyclohexyl-methyl-sulfamoyl)-phenyl]-4-oxo-butyric acid (1.96 g). LCMS: R_{T}=2.57 minutes, MS: 386 (M+H); ¹H NMR (300 MHz, DMSO-D₆) δ 0.9-1.8 (m, 10H), 2.6 (t, J=6 Hz, 2H), 2.8 (s, 3H), 3.3 (t, J=6 Hz, 2H), 3.59 (m, 1H), 7.87 (d, J=8 Hz, 1H), 8.23 (dd, J=6, 2 Hz, 1H), 8.46 (d, J=2 Hz, 1H), 12.2 (broad s, 1H).

Step 2: By proceeding in a similar manner to Example 1 (a) method A, step 5, but substituting 4-[4-chloro-3-(cyclohexyl-methyl-sulfamoyl)-phenyl]-4-oxo-butyric acid (0.5 g) for 4-(4-chloro-3-cyclohexylsulfamoyl-phenyl)-4-oxo-butyric acid and using zinc chloride (180 mg), p-toluene sulfonic acid monohydrate (250 mg), phenylhydrazine (150 mg), glacial acetic acid (8 mL), and purification is by preparative HPLC separation (mobile phase: acetonitrile-water with 0.1% TFA; gradient 10-100% over 10 minutes), there is prepared {2-[4-chloro-3-(cyclohexyl-methyl-sulfamoyl)-phenyl]-1H-indol-3-yl}-acetic acid (95 mg). LCMS: R_{T}=2.9 minutes, MS: 461 (M+H); ¹H NMR (300 MHz, CD₃OD) δ 1-1.8 (m, 10H), 2.9 (s, 3H), 3.7 (m, 1H), 3.82 (s, 2H), 7.08 (t, J=7 Hz, 1H), 7.18 (t, J=8 Hz, 1H), 7.4 (d, J=8 Hz, 1H), 7.6 (d, J=7.9 Hz, 1H), 7.7 (d, J=8.2 Hz, 1H), 7.89 (dd, J=6.0, 2.2 Hz, 1H), 8.4 (d, J=2.2 Hz, 1H). IC₅₀=346 nM

### (y) {2-[4-Chloro-3-(4-trifluoromethyl-benzylsulfamoyl)-phenyl]-1H-indol-3-yl}-acetic acid

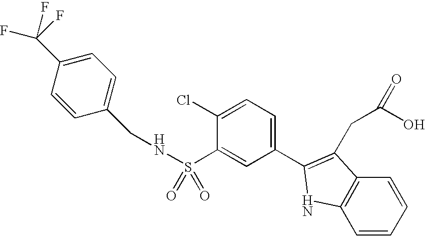

Step 1: By proceeding in a similar manner to Example 1(a) method A, step 4, but substituting 4-trifluoromethyl-benzylamine (2.8 g) for cyclohexylamine, there is prepared 4-[4-chloro-3-(4-trifluoromethyl-benzylsulfamoyl)-phenyl]-4-oxo-butyric acid (2.2 g). LCMS: R_{T}=2.54 minutes, MS: 432 (M+H); ¹H NMR (300 MHz, DMSO-D₆) δ 2.6 (t, J=6 Hz, 2H), 3.23 (t, J=6 Hz, 2H), 4.24 (broad s, 2H), 7.42 (d, J=8 Hz, 2H), 7.55 (d, J=8 Hz, 2H), 7.7 (d, J=8.4 Hz, 1H), 8.1 (dd, J=6.4 Hz, J=2 Hz, 1H), 8.3 (d, J=2 Hz, 1H), 12.2 (broad s, 1H).

Step 2: By proceeding in a similar manner to Example 1 (a) method A, step 5, but substituting 4-[4-chloro-3-(4-trifluoromethyl-benzylsulfamoyl)-phenyl]-4-oxo-butyric acid (0.56 g) for 4-(4-chloro-3-cyclohexylsulfamoyl-phenyl)-4-oxo-butyric acid and using zinc chloride (180 mg), p-toluene sulfonic acid monohydrate (250 mg), phenylhydrazine (150 mg), glacial acetic acid (8 mL), and purification is by preparative HPLC separation (mobile phase: acetonitrile-water with 0.1% TFA; gradient 10-100% over 10 minutes), there is prepared {2-[4-chloro-3-(4-trifluoromethyl-benzylsulfamoyl)-phenyl]-1H-indol-3-yl}-acetic acid (170 mg). LCMS: R_{T}=2.7 minutes, MS: 523 (M+H); ¹H NMR (300 MHz, CD₃OD) δ 3.8 (s, 2H), 4.28 (s, 2H), 7.07 (t, J=7 Hz, 1H), 7.17 (t, J=7 Hz, 1H), 7.44 (m, 5H), 7.58 (t, J=8 Hz, 2H), 7.84 (dd, J=6 Hz, J=2.2 Hz, 1H), 8.32 (d, J=2.2 Hz, 1H). IC₅₀=19 nM

### Example 2

### (a) [2-(4-Chloro-3-cyclohexylsulfamoyl-phenyl)-1-methyl-1H-indol-3-yl]-acetic acid

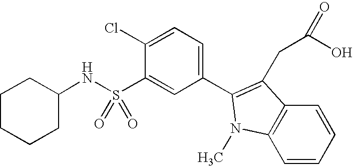

To a mixture of 4-(4-chloro-3-cyclohexylsulfamoyl-phenyl)-4-oxo-butyric acid [1.5 g, see Example 1(a), Method A, step 4], zinc chloride (550 mg), p-toluene sulfonic acid monohydrate (770 mg) in tert-butanol (100 mL) is added 1-methyl-1-phenyl hydrazine (500 mg). The mixture is heated at reflux for 20 hours, cooled to room temperature, poured into aqueous 2 N HCl (˜200 mL) and extracted twice with EtOAc. The combined organic layer is washed twice with water, dried over sodium sulfate and evaporated. The residue is crystallized from MeOH to afford [2-(4-chloro-3-cyclohexylsulfamoyl-phenyl)-1-methyl-1H-indol-3-yl]-acetic acid as a solid (1.4 g). LCMS: R_{T}=3.25 minutes, MS: 461 (M+H); ¹H NMR (300 MHz, DMSO-D₆) δ 0.8-1.7 (m, 10H) 3.04 (m, 1H) 3.51 (s, 2H) 3.61 (s, 3H) 7.1 (t, J=7.4 Hz, 1H) 7.3 (t, J=7 Hz, 1H) 7.49 (d, J=8.3 Hz, 1H) 7.56 (d, J=8 Hz, 1H) 7.74 (dd, J=6 Hz, J=2.2 Hz, 1H) 7.82 (d, J=8 Hz, 1H) 7.95 (d, J=8 Hz, 1H) 8.03 (d, J=2.1 Hz, 1H) 12.28 (broad s, 1H). IC₅₀=52 nM

### (b) [1-Benzyl-2-(4-chloro-3-cyclohexylsulfamoyl-phenyl)-1H-indol-3-yl]-acetic acid

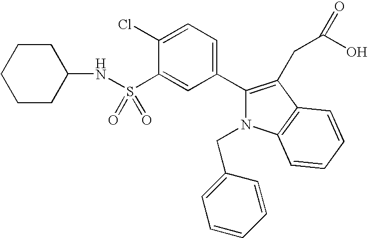 Method A:

By proceeding in a similar manner to Example 2(a), but substituting N-benzyl-N-phenylhydrazine hydrochloride (5.6 g) for 1-methyl-1-phenyl hydrazine, using 4-(4-chloro-3-cyclohexylsulfamoyl-phenyl)-4-oxo-butyric acid (6 g), zinc chloride (3.3 g), p-toluene sulfonic acid monohydrate (4.6 g) and tert-butanol (200 mL), and the purification is by silica gel flash column chromatography eluting with 20-60% EtOAc in heptane instead of recrystallization, there is prepared [1-benzyl-2-(4-chloro-3-cyclohexylsulfamoyl-phenyl)-1H-indol-3-yl]-acetic acid (7 g). LCMS: R_{T}=3.64 minutes, 537 (M+H); ¹H NMR (300 MHz, DMSO-D₆) δ ppm 0.8-1.7 (series of m, 10H) 2.9 (m, 1H) 3.54 (s, 2H) 5.33 (s, 2H) 6.82 (d, J=7 Hz, 2H) 7.16 (m, 6H) 7.43 (d, J=8 Hz, 1H) 7.61 (d, J=8 Hz, 1H) 7.77 (d, J=8 Hz, 1H) 7.93 (d, J=8.1 Hz, 1H) 8 (d, J=2 Hz, 1H) 12.3 (broad s, 1H). IC₅₀=42 nM

Method B:

By proceeding in a similar manner to Example 1 (a) method A, step 5, but substituting N-benzyl-N-phenylhydrazine hydrochloride (0.61 g) for phenyl hydrazine, using 4-(4-chloro-3-cyclohexylsulfamoyl-phenyl)-4-oxo-butyric acid (0.75 g), p-toluene sulfonic acid (0.48 g), zinc chloride (0.34 g) and glacial acetic acid (4 mL), and the reaction temperature is at 160° C., there is prepared [1-benzyl-2-(4-chloro-3-cyclohexylsulfamoyl-phenyl)-1H-indol-3-yl]-acetic acid (410 mg).

### (c) {2-[4-Chloro-3-(piperidine-1-sulfonyl)-phenyl]-1-methyl-1H-indol-3-yl}-acetic acid

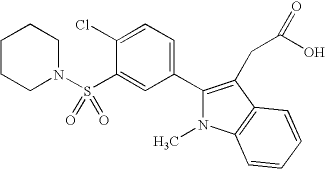

By proceeding in a similar manner to Example 1 (a) method A, step 5, but substituting 4-[4-chloro-3-(piperidine-1-sulfonyl)-phenyl]-4-oxo-butyric acid [0.36 g, see Example 1 (p), step 1] for 4-(4-chloro-3-cyclohexylsulfamoyl-phenyl)-4-oxo-butyric acid, substituting N-methyl-N-phenyl-hydrazine (0.125 g) for phenyl hydrazine, using p-toluene sulfonic acid (0.19 g), zinc chloride (0.14 g) and glacial acetic acid (2 mL), and the reaction temperature is at 160° C., there is prepared {2-[4-chloro-3-(piperidine-1-sulfonyl)-phenyl]-1-methyl-1H-indol-3-yl}-acetic acid (24 mg). LCMS: R_{T}=3.27 minutes, MS: 447 (M+H); ¹H NMR (300 MHz, CDCl₃) δ ppm 1.6 (m, 6H) 3.3 (m, 4H) 3.63 (s, 3H) 3.67 (s, 2H) 7.15-7.4 (m, 3H) 7.63 (m, 3H) 8.13 (s, 1H).

### Example 3

### (a) (S)-2-{2-[2-(4-Chloro-3-cyclohexylsulfamoyl-phenyl)-1H-indol-3-yl]-acetylamino}-3-methyl-butyric acid

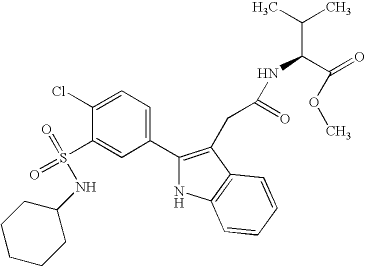

A mixture of [2-(4-chloro-3-cyclohexylsulfamoyl-phenyl)-1H-indol-3-yl]-acetic acid [200 mg, Example 1 (a)], HBTU (200 mg), and DIEA (130 mg) in DCM (20 mL) is stirred at room temperature for 16 hours, followed by the addition of (S)-2-amino-3-methyl-butyric acid methyl ester (168 mg). After stirring for 6 hours at room temperature, the reaction mixture is diluted with DCM, washed with aqueous 2 HCl and water, dried over sodium sulfate, and concentrated in vacuo. The residue is purified by a short silica gel column chromatography eluting with 50-100% EtOAc in heptane and 5% MeOH in EtOAc to afford (S)-2-{2-[2-(4-chloro-3-cyclohexylsulfamoyl-phenyl)-1H-indol-3-yl]-acetylamino }-3-methyl-butyric acid methyl ester (140 mg). LCMS: R_{T}=2.97 minutes, MS: 560 (M+H). IC₅₀=160 nM

### (b) (S)-2-{2-[2-(4-Chloro-3-cyclohexylsulfamoyl-phenyl)-1H-indol-3-yl]-acetylamino}-3-methyl-butyric acid

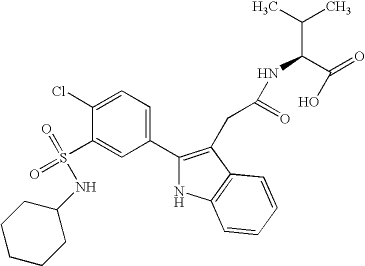

A mixture of (S)-2-{2-[2-(4-chloro-3-cyclohexylsulfamoyl-phenyl)-1H-indol-3-yl]-acetylamino}-3-methyl-butyric acid methyl ester [120 mg, Example 3(a)], lithium hydroxide (100 mg) in MeOH (20 mL) and water (10 mL) is heated at 70° C. for 6 hours, cooled to room temperature, diluted with EtOAc, washed with aqueous 2 N HCl and water, dried over sodium sulfate, and concentrated in vacuo. The residue is purified by a short silica gel column chromatography eluting with 0-20% MeOH in DCM to afford (S)-2-{2-[2-(4-chloro-3-cyclohexylsulfamoyl-phenyl)-1H-indol-3-yl]-acetylamino}-3-methyl-butyric acid as a solid (55 mg). LCMS: R_{T}=2.69 minutes, MS: 546 (M+H); ¹H NMR (300 MHz, DMSO-D₆) δ ppm 0.7-1.7 (m, 16H) 2.06 (m, 1H) 3.04 (m, 1H) 3.64 (d, J=15.5 Hz, 1H) 3.8 (d, J=15.3 Hz, 1H) 4.08 (m, 1H) 7 (t, J=7.7 Hz, 1H) 7.13 (t, J=8 Hz, 1H) 7.38 (d, J=8.2 Hz, 1H) 7.66 (d, J=8 Hz, 1H) 7.73 (d, J=8.3 Hz, 1H) 7.9 (d, J=8 Hz, 1H) 8 (broad s, 1H) 8.14 (dd, J=6.2 Hz, J=2 Hz, 1H) 8.32 (d, J=2 Hz, 1H) 11.49 (s, 1H).

### Example 4

### [2-(4-Chloro-3-cyclohexylsulfamoyl-phenyl)-1H-indol-3-yl]-acetic acid 2-dimethylamino-ethyl ester

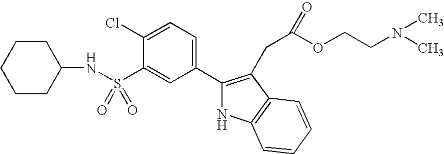

A mixture of [2-(4-chloro-3-cyclohexylsulfamoyl-phenyl)-1H-indol-3-yl]-acetic acid [200 mg, Example 1 (a)], HBTU (200 mg), and DIEA (130 mg) in DCM (20 mL) is stirred at room temperature for 16 hours, followed by the addition of 2-dimethylamino-ethanol (90 mg). After stirring at room temperature for 6 hours, the reaction mixture is diluted with DCM, washed with aqueous 2 N HCl and water, dried over sodium sulfate, and concentrated in vacuo. The residue is purified by a short silica gel column chromatography eluting with 50 to 100% EtOAc in heptane and 5% MeOH in EtOAc to afford [2-(4-chloro-3-cyclohexylsulfamoyl-phenyl)-1H-indol-3-yl]-acetic acid 2-dimethylamino-ethyl ester (140 mg). LCMS: R_{T}=2.24 minutes, MS: 518 (M+H); ¹H NMR (300 MHz, DMSO-D₆) δ 0.8-1.7 (m, 10H) 2.1 (s, 6H) 2.44 (m, 2H) 3.04 (m, 1H) 3.84 (s, 2H) 4.1 (t, J=6 Hz, 2H) 7.04 (t, J=7 Hz, 1H) 7.16 (t, J=7 Hz, 1H) 7.4 (d, J=8 Hz, 1H) 7.56 (d, J=8 Hz, 1H) 7.77 (d, J=8.3 Hz, 1H) 7.87 (dd, J=6 Hz, J=2.3 Hz, 1H) 7.95 (d, J=8.2 Hz, 1H) 8.23 (d, J=2.2 Hz, 1H) 11.59 (s, 1H).

### Example 5

### 2-Chloro-N-cyclohexyl-5-[3-(5-oxo-4,5-dihydro-1,3,4-oxadiazol-2-ylmethyl)-1H-indol-2-yl]-benzenesulfonamide

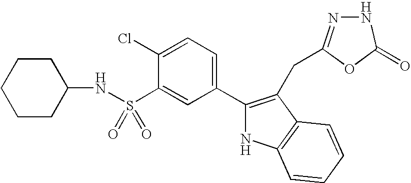

A mixture of [2-(4-chloro-3-cyclohexylsulfamoyl-phenyl)-1H-indol-3-yl]-acetic acid [300 mg, Example 1 (a)], HBTU (300 mg), and DIEA (210 mg) in DCM (30 mL) is stirred at room temperature for 4 hours, followed by the addition of hydrazine hydrate (350 mg). After stirring at room temperature for 20 hours, the reaction mixture is diluted with DCM, washed with aqueous 2 N HCl and water, dried over sodium sulfate, and concentrated in vacuo. The residue is dissolved in 1,4-dioxane and CDI (450 mg) is added. The reaction mixture is heated at reflux for 6 hours, cooled to room temperature and let it stay overnight. The reaction mixture is diluted with EtOAc, washed with aqueous 2 N HCl and water, dried over sodium sulfate, and concentrated in vacuo. The residue is purified by a short silica gel column chromatography eluting with 50-75% EtOAc in heptane to afford 2-chloro-N-cyclohexyl-5-[3-(5-oxo-4,5-dihydro-1,3,4-oxadiazol-2-ylmethyl)-1H-indol-2-yl]-benzenesulfonamide (70 mg). LCMS: R_{T}=3.17 minutes, MS: 487 (M+H); ¹H NMR (300 MHz, DMSO-D₆) δ 0.8-1.7 (m, 10H) 3.04 (m, 1H) 4.13 (s, 2H) 7.08 (t, J=7.2 Hz, 1H) 7.2 (t, J=7.5 Hz, 1H) 7.44 (d, J=8 Hz, 1H) 7.58 (d, J=8 Hz, 1H) 7.81 (d, J=8.3 Hz, 1H) 7.87 (dd, J=6.2 Hz, J=2 Hz, 1H) 7.95 (d, J=8 Hz, 1H) 8.23 (d, J=2.1 Hz, 1H) 11.7 (s, 1H) 12.1 (broad s, 1H). IC₅₀=33 DM

### Example 6

### 5-[3-(2-Benzenesulfonylamino-2-oxo-ethyl)-1H-indol-2-yl]-2-chloro-N-cyclohexyl benzenesulfonamide

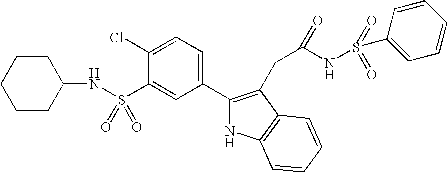

A mixture of [2-(4-chloro-3-cyclohexylsulfamoyl-phenyl)-1H-indol-3-yl]-acetic acid [300 mg, Example 1 (a)], HBTU (256 mg), and DIEA (DIEA, 180 mg) in DCM (30 mL) is stirred at room temperature for 16 hours, and concentrated in vacuo. The residue is azeotroped twice with toluene and is used in the next reaction (intermediate A). To a mixture of benzene sulfonamide (550 mg) in toluene (30 mL) is added trimethyl aluminum (2N in toluene, 1.8 mL) dropwise at 0° C. After the gas evolution finishes, the suspension is heated at reflux for 2 hours and a solution of the intermediate A in a mixture of toluene and anhydrous THF (1:1, 20 mL) is added. The resulting mixture is heated at reflux for 4 hours. The reaction mixture is quenched with water, acidified with aqueous 2 N HCl, and extracted twice with EtOAc. The combined organic layer is washed with water, dried over sodium sulfate, and concentrated in vacuo. The residue is purified by a silica gel column chromatography eluting with 50-80% EtOAc in heptane to afford 5-[3-(2-benzenesulfonylamino-2-oxo-ethyl)-1H-indol-2-yl]-2-chloro-N-cyclohexyl-benzenesulfonamide as a solid (130 mg). LCMS: R_{T}=2.87 minutes, MS: 586 (M+H); ¹H NMR (300 MHz, DMSO-D₆) δ 0.8-1.8 (m, 10H) 3 (m, 1H) 3.76 (s, 2H) 6.95 (t, J=8 Hz, 1H) 7.1 (t, J=8 Hz, 1H) 7.36 (m, 5H) 7.5-7.9 (series of m, 5H) 8.1 (d, J=2 Hz, 1H) 11.5 (s, 1H) 12.4 (broad s, 1H). IC₅₀=5 nM

### Example 7

### (a) 2-[2-(4-Chloro-3-cyclohexylsulfamoyl-phenyl)-1H-indol-3-yl]-acetamide

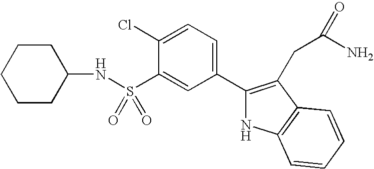

Step 1: To tetrafluorophenol resin (TFP, 50 mg, 1 mmol/g) swelled in anhydrous DMF (1 mL) is added [2-(4-chloro-3-cyclohexylsulfamoyl-phenyl)-1H-indol-3-yl]-acetic acid [55 mg, Example 1(a)], and diisopropylcarbodiimide (30 mg). The mixture is shaken at room temperature for 20 hr, the resin is washed twice with DMF (2 mL), twice with THF (2 mL) and twice with DCM (2 mL), and dried under vacuum.

Step 2: The above resin from Step 1 (30 mg) is swelled in anhydrous DCM (0.5 mL) and 7 N ammonia in MeOH (2 mL) is added. The suspension is left at room temperature for 20 hours, the resin is filtered, washed twice with MeOH (2 mL) and the combined filtrate and washings are concentrated in vacuo. The residue is purified by a short silica gel column chromatography eluting with 20-60% EtOAc in heptane to afford 2-[2-(4-chloro-3-cyclohexylsulfamoyl-phenyl)-1H-indol-3-yl]-acetamide (2 mg). LCMS: R_{T}=2.92 minutes, MS: 446 (M+H). IC₅₀=132 nM

### (b) 2-[2-(4-Chloro-3-cyclohexylsulfamoyl-phenyl)-1-methyl-1H-indol-3-yl]-acetamide

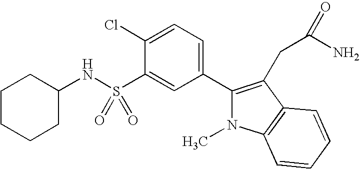

By proceeding in a similar manner to Example 7(a), but at step 1 substituting [2-(4-chloro-3-cyclohexylsulfamoyl-phenyl)-1-methyl-1H-indol-3-yl]-acetic acid [0.55 g, Example 2(a)] for [2-(4-chloro-3-cyclohexylsulfamoyl-phenyl)-1H-indol-3-yl]-acetic acid, there is prepared 2-[2-(4-chloro-3-cyclohexylsulfamoyl-phenyl)-1-methyl-1H-indol-3-yl]-acetamide as a solid (7 mg). LCMS: R_{T}=3.07 minutes, MS: 460 (M+H).

### Example 8

### [2-(4-Chloro-3-cyclohexylsulfamoyl-phenyl)-1H-indol-3-yl]-acetic acid methyl ester

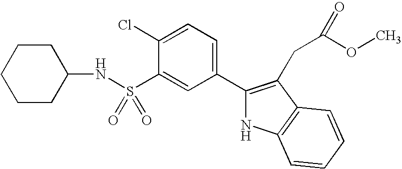

A mixture of [2-(4-chloro-3-cyclohexylsulfamoyl-phenyl)-1H-indol-3-yl]-acetic acid [200 mg, Example 1 (a)], and HBTU (255 mg) in DCM (50 mL) is stirred at room temperature for 16 hours, followed by the addition of anhydrous MeOH (1 mL). After stirring at room temperature for 22 hours, the reaction mixture is diluted with DCM, washed with water, dried over sodium sulfate, and concentrated in vacuo. The residue is purified by a short silica gel column chromatography eluting with 25-40% EtOAc in heptane to afford [2-(4-chloro-3-cyclohexylsulfamoyl-phenyl)-1H-indol-3-yl]-acetic acid methyl ester as a solid (80 mg). LCMS: R_{T}=3.39 minutes, MS: 461 (M+H); ¹H NMR (300 MHz, CDCl₃) δ 1-1.9 (m, 10H) 3.2 (m, 1H) 3.73 (s, 3H) 3.83 (s, 2H) 4.97 (d, J=7.8 Hz, 1H) 7.2 (m, 2H) 7.40 (d, J=7.8 Hz, 1H) 7.62 (d, J=8.2 Hz, 1H) 7.68 (d, J=7.8 Hz, 1H) 7.91 (dd, J=6.3 Hz, J=2 Hz, 1H) 8.3 (s, 1H) 8.35 (d, J=2 Hz, 1H).

### Example 9

### 2-Chloro-N-cyclohexyl-5-[3-(2-hydroxy-ethyl)-1-methyl-1H-indol-2-yl]-benzenesulfonamide

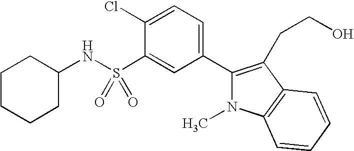

To a solution of [2-(4-chloro-3-cyclohexylsulfamoyl-phenyl)-1-methyl-1H-indol-3-yl]-acetic acid [100 mg, Example 2(a)] in anhydrous THF (5 mL) cooled to 0° C. is added 1 M solution of lithium aluminum hydride in THF (0.25 mL) and the mixture is stirred for 30 minutes while warming up to room temperature. The reaction mixture is quenched with anhydrous MeOH, diluted with 1 N aqueous HCl (5 mL), and extracted with EtOAc. The organic layer is dried over sodium sulfate and concentrated in vacuo. The residue is chromatographed on a prepacked silica gel column eluting with EtOAc to afford 2-chloro-N-cyclohexyl-5-[3-(2-hydroxy-ethyl)-1-methyl-1H-indol-2-yl]-benzenesulfonamide (12 mg). LCMS: R_{T}=3.37 minutes, MS: 447 (M+H). IC₅₀=8713 nM

### Example 10

### (a) [2-(4-Chloro-3-cyclohexylsulfamoyl-phenyl)-5-methoxy-1H-indol-3-yl]-acetic acid

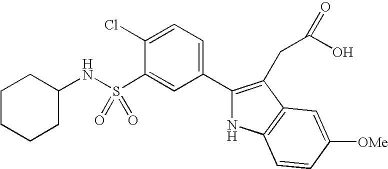 Method A:

A warm (80° C.) solution of 4-(4-chloro-3-cyclohexylsulfamoyl-phenyl)-4-oxo-butyric acid [1.86 g, see Example 1 (a), step 4] and potassium hydroxide (0.294 g) in water (20 mL) is added to a solution of 4-methoxyphenylhydrazine hydrochloride (1 g) and potassium hydroxide (0.322 g) in water (20 mL). The mixture is refluxed for 4 hours, standing at room temperature 20 hours, followed by evaporation. The residue is dissolved in glacial acetic acid (60 mL) and refluxed for 1 hour. Approximately 150 mL of water is added, and the mixture is stirred at room temperature for 1 hour, followed by the filtration of the solid precipitation. The solid is dissolved in EtOAc, the resulting solution is treated with charcoal, followed by filtration. The filtrate is concentrated in vacuo and the residue is crystallized with diisopropylether. The resulted material is subjected to medium pressure liquid chromatography (MPLC) on a commercially available Flash silica gel column known as ISOLUTE® (available from Separtis GmbH, Germany) with an eluent-mixture of EtOAc:n-heptane:DCM:MeOH: 28-30% aqueous ammonia (volume parts in the order: 10:5:5:5:1) to afford [2-(4-chloro-3-cyclohexylsulfamoyl-phenyl)-5-methoxy-1H-indol-3-yl]-acetic acid (3.5 mg). LCMS: R_{T}=3.07 minutes, MS: 477 (M+H); ¹H NMR (300 MHz, DMSO-D₆) δ 0.8-1.7 (m, 10H) 3.04 (m, 1H) 3.7 (s, 2H) 3.78 (s, 3H) 6.84 (d, J=8 Hz, 1H) 7.02 (s, 1H) 7.28 (d, J=7.5 Hz, 1H) 7.78 (d, J=7.5 Hz, 1H) 7.9 (m, 2H) 8.22 (s, 1H) 11.4 (s, 1H) 12.1 (broad s, 1H). IC₅₀=3 nM

Method B:

Step 1. A mixture of 2-chloronitrobenzene (53 g, 0.34 mol), iron (1.5 g) and bromine (23 mL, 0.45 mol) is stirred at reflux under N₂ for 20 hours. The reaction is concentrated and the residue is purified by flash chromatography on silica gel eluting with 10% EtOAc-heptane. The appropriate fractions are concentrated, filtered, and rinsed with ethanol, and dried. The solid is recrystallized from ethanol to afford 5-bromo-2-chloronitrobenzene (37.9 g). After storage of the mother liquors at 0° C. overnight, a second crop of product is isolated and dried to afford an additional 5-bromo-2-chloronitrobenzene (7 g). MS: 235 (M+H); m.p. 65-67° C.

Step 2. A solution of 5-bromo-2-chloronitrobenzene (10.3 g, 43.6 mmol) in EtOAc (200 mL) is hydrogenated over Raney nickel (6 g of 50% in H₂O) at 55 psi H₂ for 5 hours. The mixture is filtered through a bed of celite and rinsed with EtOAc. The filtrate is treated with ethereal HCl (60 mL, 1 M solution in Et₂O) under N₂. The resulting suspension is stirred for 1 hour and Et₂O (100-200 mL) is added. The mixture is filtered to afford 5-bromo-2-chloroaniline hydrochloride (4.85 g) as a solid. MS: 205 (M+H); m.p. 152-155° C.

Step 3. A suspension of 5-bromo-2-chloroaniline hydrochloride (41.4 g, 0.17 mol) in CH₃CN (380 mL) is cooled to 5° C. and concentrated HCl (277 mL) is added over 10 minutes. The suspension is cooled to −5° C. and a solution of NaNO₂ (14.2 g, 0.21 mol) in H₂O (40 mL) is added dropwise over 10-15 minutes. The mixture is stirred for additional 5 minutes and 30% (w/w) SO₂ in HOAc (435 mL) is added at 0° C., followed by an addition of a solution of copper(II) chloride dihydrate (15.3 g, 0.09 mol) in H₂O (40 mL). The reaction is stirred at room temperature for 1.5 hours. The reaction mixture is filtered and the solid is dried to afford 5-bromo-2-chlorobenzenesulfonyl chloride (18.4 g). The filtrate is stored at 0° C. for 18 hours. The precipitate is collected and dried to afford additional 5-bromo-2-chlorobenzenesulfonyl chloride (9.6 g). MS: 288 (M+H).

Step 4. A reaction flask is charged with cyclohexylamine (15 mL, 131 mmol), DIEA (30 mL, 172 mmol) and CH₂Cl₂ (150 mL). The mixture is cooled to −5° C. under N₂ and a solution of 5-bromo-2-chlorobenzenesulfonyl chloride (25 g, 86.2 mmol) in CH₂Cl₂ (200 mL) is added dropwise over 45 minutes. The mixture is stirred at room temperature for 20 hours, cooled to −10° C. and 2 HCl (150 mL) is added. The organic layer is washed with 2 HCl (2×150 mL) and H₂O (150 mL), dried (Na₂SO₄) and concentrated to afford 5-bromo-2-chloro-N-cyclohexylbenzenesulfonamide 30 g (99%) as a solid. MS: 351 (M+H).

Step 5. To a solution of 1-(tert-butoxycarbonyl)-5-methoxy-1H-indol-2-ylboronic acid (867 mg), 5-bromo-2-chloro-N-cyclohexyl-benzenesulfonamide [700 mg, Intermediate (2)] and CsF (420 mg) in dioxane-H₂O (20 mL, 10:1) is added PdCl₂(dppf)₂ (162 mg) at room temperature under N₂. The reaction is heated to 80° C. and stirred for 2 hr. The reaction mixture is concentrated in vacuo. The residue is dissolved in EtOAc and filtered through a short silica column. The filtrate is concentrated in vacuo and the residue is purified by flash chromatography on silica gel eluting with 5% to 50% EtOAc in heptane to afford 2-(4-chloro-3-cyclohexylsulfamoyl-phenyl)-5-methoxy-indole-1-carboxylic acid tert-butyl ester as a solid (650 mg). LCMS: R_{T}=3.61 minutes, MS: 519 (M+H).

Step 6. TFA (3 mL) is added to a solution of 2-(4-chloro-3-cyclohexylsulfamoyl-phenyl)-5-methoxy-indole-1-carboxylic acid tert-butyl ester (640 mg) in DCM (6 mL). The reaction mixture is stirred at room temperature overnight. The mixture is concentrated in vacuo. The residue is dissolved in EtOAc and washed with 1 N NaHCO₃. The organic layer is separated, dried over MgSO₄ and concentrated to afford 2-chloro-N-cyclohexyl-5-(5-methoxy-1H-indol-2-yl)-benzenesulfonamide as a solid (496 mg). LCMS: R_{T}=3.17 minutes, MS: 419 (M+H).

Step 7. Oxalyl chloride (0.15 mL) is slowly added to a solution of 2-chloro-N-cyclohexyl-5-(5-methoxy-1H-indol-2-yl)-benzenesulfonamide (480 mg) in DCM (111 mL) at room temperature. After stirring for 3 hr, MeOH (3 mL) is added and stirred for 15 minutes. The mixture is concentrated. The residue is purified by flash chromatography on silica gel eluting with 10% to 50% EtOAc in heptane to afford [2-(4-chloro-3-cyclohexylsulfamoyl-phenyl)-5-methoxy-1H-indol-3-yl]-oxo-acetic acid methyl ester as a solid (390 mg). LCMS: R_{T}=2.8 minutes, MS: 505 (M+H).

Step 8. Triethylsilane (0.24 mL) is slowly added to a solution of [2-(4-chloro-3-cyclohexylsulfamoyl-phenyl)-5-methoxy-1H-indol-3-yl]-oxo-acetic acid methyl ester (380 mg) in TFA (4 mL) at room temperature. After stirring for 5 hr, the volatile is removed in vacuo. The residue is dissolved in EtOAc and washed with 1 N NaHCO₃. The organic layer is separated, dried over MgSO₄ and concentrated. The residue is purified by flash chromatography on silica gel eluting with 5% to 40% EtOAc in heptane to afford [2-(4-chloro-3-cyclohexylsulfamoyl-phenyl)-5-methoxy-1H-indol-3-yl]-acetic acid methyl ester as a solid (123 mg). LCMS: R_{T}=3.07 minutes, MS: 491 (M+H); ¹H NMR (300 MHz, CDCl₃) δ 1.16-1.29 (m, 5H), 1.49-1.8 (m, 5H), 3.2 (m, 1H), 3.73 (s, 3H), 3.79 (s, 2H), 3.87 (s, 3H), 5.1 (d, J=7.8 Hz, 1H), 6.89 (dd, J=8.7, 2.4 Hz, 1H), 7.08 (d, J=2.4 Hz, 1H), 7.26 (d, J=9 Hz, 1H), 7.57 (d, J=8.1 Hz, 1H), 7.85 (dd, J=8.4, 2.4 Hz, 1H), 8.34 (d, J=2.1 Hz, 1H), 8.52 (s, 1H).

Step 9. To a solution of [2-(4-chloro-3-cyclohexylsulfamoyl-phenyl)-5-methoxy-1H-indol-3-yl]-acetic acid methyl ester (30 mg) in MeOH/H₂O (1:1, 0.6 mL) is added lithium hydroxide monohydrate (5 mg). The reaction mixture is stirred at 70° C. for 3 hr. EtOAc (15 mL) is added and the solution is washed with 1 N HCl (10 mL). The organic layer is separated, dried over MgSO₄ and concentrated to afford [2-(4-chloro-3-cyclohexylsulfamoyl-phenyl)-5-methoxy-1H-indol-3-yl]-acetic acid as a solid (25 mg). LCMS: R_{T}=2.85 minutes, MS: 477 (M+H); ¹H NMR (300 MHz, CD₃OD) δ 1.23-1.3 (m, 5H), 1.51-1.74 (m, 5H), 3.06-3.16 (m, 1H), 3.79 (s, 2H), 3.83 (s, 3H), 6.83 (dd, J=8.7, 2.4 Hz, 1H), 7.08 (d, J=2.4 Hz, 1H), 7.29 (d, J=8.7 Hz, 1H), 7.67 (d, J=8.1 Hz, 1H), 7.88 (dd, J=8.4, 2.4 Hz, 1H), 8.37 (d, J=1.8 Hz, 1H).

### (b) [5-Chloro-2-(4-chloro-3-cyclohexylsulfamoyl-phenyl)-1H-indol-3-yl]-acetic acid

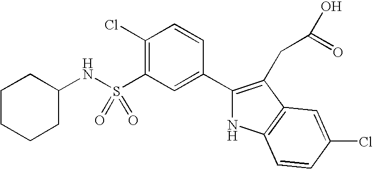

Step 1. By proceeding in a similar manner to Example 10(a), method B, step 5, but substituting 1-(tert-butoxycarbonyl)-5-chloro-1H-indol-2-ylboronic acid (700 mg) for 1-(tert-butoxycarbonyl)-5-methoxy-1H-indol-2-ylboronic acid and using 5-bromo-2-chloro-N-cyclohexyl-benzenesulfonamide (631 mg), there is prepared 5-chloro-2-(4-chloro-3-cyclohexylsulfamoyl-phenyl)-indole-1-carboxylic acid tert-butyl ester as a solid (557 mg).

Step 2. By proceeding in a similar manner to Example 10(a), method B, step 6, but substituting 5-chloro-2-(4-chloro-3-cyclohexylsulfamoyl-phenyl)-indole-1-carboxylic acid tert-butyl ester (557 mg) for 2-(4-chloro-3-cyclohexylsulfamoyl-phenyl)-5-methoxy-indole-1-carboxylic acid tert-butyl ester, there is prepared 2-chloro-5-(5-chloro-1H-indol-2-yl)-N-cyclohexyl-benzenesulfonamide as a solid (370 mg).

Step 3. By proceeding in a similar manner to Example 10(a), method B, step 7, but substituting 2-chloro-5-(5-chloro-1H-indol-2-yl)-N-cyclohexyl-benzenesulfonamide (370 mg) for 2-chloro-N-cyclohexyl-5-(5-methoxy-1H-indol-2-yl)-benzenesulfonamide, there is prepared [5-chloro-2-(4-chloro-3-cyclohexylsulfamoyl-phenyl)-1H-indol-3-yl]-oxo-acetic acid methyl ester as a solid (200 mg). LCMS: R_{T}=3.04 minutes, MS: 509 (M+H).

Step 4. By proceeding in a similar manner to Example 10(a), method B, step 8, but substituting [5-chloro-2-(4-chloro-3-cyclohexylsulfamoyl-phenyl)-1H-indol-3-yl]-oxo-acetic acid methyl ester (170 mg) for [2-(4-chloro-3-cyclohexylsulfamoyl-phenyl)-5-methoxy-1H-indol-3-yl]-oxo-acetic acid methyl ester, there is prepared [5-chloro-2-(4-chloro-3-cyclohexylsulfamoyl-phenyl)-1H-indol-3-yl]-acetic acid methyl ester as a solid (80 mg). LCMS: R_{T}=3.39 minutes, MS: 495 (M+H); ¹H NMR (300 MHz, CD₃OD) δ 1.23-1.3 (m, 5H), 1.51-1.74 (m, 5H), 3.06-3.16 (m, 1H), 3.73 (s, 3H), 3.81 (s, 2H), 7.14 (dd, J=8.7, 2.1 Hz, 1H), 7.36 (d, J=8.7 Hz, 1H), 7.57 (d, J=1.5 Hz, 1H), 7.71 (d, J=8.4 Hz, 1H), 7.86 (dd, J=8.4, 2.4 Hz, 1H), 8.35 (d, J=2.4 Hz, 1H).

Step 5. By proceeding in a similar manner to Example 10(a), method B, step 9, but substituting [5-chloro-2-(4-chloro-3-cyclohexylsulfamoyl-phenyl)-1H-indol-3-yl]-acetic acid methyl ester (75 mg) for [2-(4-chloro-3-cyclohexylsulfamoyl-phenyl)-5-methoxy-1H-indol-3-yl]-acetic acid methyl ester, there is prepared [5-chloro-2-(4-chloro-3-cyclohexylsulfamoyl-phenyl)-1H-indol-3-yl]-acetic acid as a solid (70 mg). LCMS: R_{T}=2.85 minutes, MS: 481 (M+H); ¹H NMR (300 MHz, CD₃OD) δ 1.09-1.33 (m, 5H), 1.51-1.74 (m, 5H), 3.07-3.16 (m, 1H), 3.79 (s, 2H), 7.13 (dd, J=8.4, 1.8 Hz, 1H), 7.36 (d, J=8.7 Hz, 1H), 7.58 (d, J=2.1 Hz, 1H), 7.71 (d, J=8.1 Hz, 1H), 7.89 (dd, J=8.1, 2.1 Hz, 1H), 8.37 (d, J=2.1 Hz, 1H).

### (c) [2-(4-Chloro-3-cyclohexylsulfamoyl-phenyl)-5-hydroxy-1H-indol-3-yl]-acetic acid

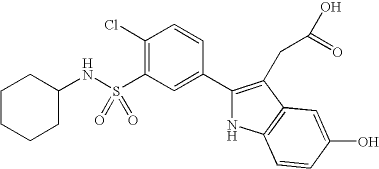

To a solution of [2-(4-chloro-3-cyclohexylsulfamoyl-phenyl)-5-methoxy-1H-indol-3-yl]-acetic acid [80 mg, Example 10(a)] in DCM (2 mL), boron tribromide (0.335 mL, IM in DCM) is added. The reaction is stirred at room temperature for 18 hr. EtOAc (10 mL) and 1 N NaHCO₃ (10 mL) are added. The organic layer is separated, dried over MgSO₄ and concentrated to afford [2-(4-chloro-3-cyclohexylsulfamoyl-phenyl)-5-hydroxy-1H-indol-3-yl]-acetic acid as a solid (8 mg). LCMS: R_{T}=2.1 minutes, MS: 463 (M+H); ¹H NMR (300 MHz, CD₃OD) δ 1.09-1.35 (m, 5H), 1.51-1.74 (m, 5H), 3.07-3.16 (m, 1H), 3.76 (brs, 2H), 6.74 (dd, J=8.7, 1.8 Hz, 1H), 6.97 (m, 1H), 7.23 (d, J=8.4 Hz, 1H), 7.67 (d, J=8.1 Hz, 1H), 7.88 (d, J=6.3 Hz, 1H), 8.36 (m, 1H). IC₅₀=4.2 nM

### (d) [6-Chloro-2-(4-chloro-3-cyclohexylsulfamoyl-phenyl)-1H-indol-3-yl]-acetic acid

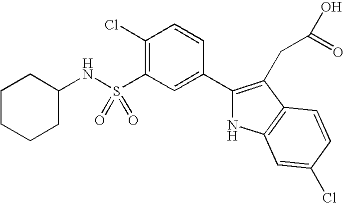

Step 1. Di-tert-butyl dicarbonate (15.8 g) is added to a solution of 6-chloroindole (10 g) and 4-(dimethylamino) pyridine (0.91 g) in DCM (330 mL). The resulting mixture is stirred at room temperature for 4 hr. The reaction mixture is washed with 1 N HCl (100 mL) and 1 N NaHCO₃ (100 mL). The organic layer is separated, dried over MgSO₄ and concentrated. The crude is recrystallized from heptane/ether to afford 6-chloro-indole-1-carboxylic acid tert-butyl ester (14.9 g). Step 2. To a solution of 6-chloro-indole-1-carboxylic acid tert-butyl ester (2 g) in dry THF (10 mL) is added triisopropyl borate (2.74 mL) under N₂. The mixture is cooled to 0° C. in an ice bath. Lithium diisopropylamine (4.97 mL, 2 M) is added over an hour at 0° C. The reaction is stirred at 0° C. for 30 minutes. 2 N HCl (10 mL) is added. The resulting mixture is extracted with EtOAc. The organic layer is dried, filtered and concentrate. The residue is purified by flash chromatography on silica gel eluting with 5% to 60% EtOAc in heptane to afford 1-(tert-butoxycarbonyl)-6-chloro-1H-indol-2-ylboronic acid as a solid (1 g).

Step 3. By proceeding in a similar manner to Example 10(a), method B, step 5, but substituting 1-(tert-butoxycarbonyl)-6-chloro-1H-indol-2-ylboronic acid (502 mg) for 1-(tert-butoxycarbonyl)-5-methoxy-1H-indol-2-ylboronic acid and using 5-bromo-2-chloro-N-cyclohexyl-benzenesulfonamide [500 mg, Intermediate (2)], there is prepared 6-chloro-2-(4-chloro-3-cyclohexylsulfamoyl-phenyl)-indole-1-carboxylic acid tert-butyl ester as a solid (429 mg).

Step 4. By proceeding in a similar manner to Example 10(a), method B, step 6, but substituting 6-chloro-2-(4-chloro-3-cyclohexylsulfamoyl-phenyl)-indole-1-carboxylic acid tert-butyl ester (557 mg) for 2-(4-chloro-3-cyclohexylsulfamoyl-phenyl)-5-methoxy-indole-1-carboxylic acid tert-butyl ester, there is prepared 2-chloro-5-(6-chloro-1H-indol-2-yl)-N-cyclohexyl-benzenesulfonamide as a solid (480 mg).

Step 5. By proceeding in a similar manner to Example 10(a), method B, step 7, but substituting 2-chloro-5-(6-chloro-1H-indol-2-yl)-N-cyclohexyl-benzenesulfonamide (480 mg) for 2-chloro-N-cyclohexyl-5-(5-methoxy-1H-indol-2-yl)-benzenesulfonamide, there is prepared [6-chloro-2-(4-chloro-3-cyclohexylsulfamoyl-phenyl)-1H-indol-3-yl]-oxo-acetic acid methyl ester as a solid (210 mg). LCMS: R_{T}=2.77 minutes, MS: 509 (M+H).

Step 6. By proceeding in a similar manner to Example 10(a), method B, step 8, but substituting [6-chloro-2-(4-chloro-3-cyclohexylsulfamoyl-phenyl)-1H-indol-3-yl]-oxo-acetic acid methyl ester (200 mg) for [2-(4-chloro-3-cyclohexylsulfamoyl-phenyl)-5-methoxy-1H-indol-3-yl]-oxo-acetic acid methyl ester, there is prepared [6-chloro-2-(4-chloro-3-cyclohexylsulfamoyl-phenyl)-1H-indol-3-yl]-acetic acid methyl ester as a solid (189 mg).

Step 7. By proceeding in a similar manner to Example 10(a), method B, step 9, but substituting [6-chloro-2-(4-chloro-3-cyclohexylsulfamoyl-phenyl)-1H-indol-3-yl]-acetic acid methyl ester (189 mg) for [2-(4-chloro-3-cyclohexylsulfamoyl-phenyl)-5-methoxy-1H-indol-3-yl]-acetic acid methyl ester, there is prepared [6-chloro-2-(4-chloro-3-cyclohexylsulfamoyl-phenyl)-1H-indol-3-yl]-acetic acid as a solid (151 mg). LCMS: R_{T}=2.83 minutes, MS: 481 (M+H); ¹H NMR (300 MHz, CD₃OD) δ 1.09-1.35 (m, 5H), 1.51-1.74 (m, 5H), 3.11 (m, 1H), 3.81 (brs, 2H), 7.05 (d, J=6.9 Hz, 1H), 7.39 (m, 1H), 7.55 (m, 1H), 7.69 (m, 1H), 7.87 (m, 1H), 8.37 (m, 1H), 11.17 (brs, 1H). IC₅₀=1 nM

### (e) {2-[3-(Cyclohexyl-methyl-sulfamoyl)-phenyl]-1H-indol-3-yl}-acetic acid

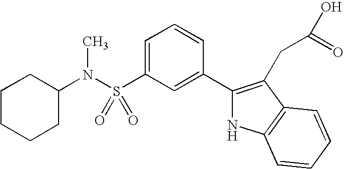

Step 1. 3-Bromo-benzenesulfonyl chloride (2.82 mL) is slowly added to a solution of cyclohexyl-methyl-amine (3 mL) and DIEA (5.11 mL) in DCM (40 mL) at 0° C. The resulting mixture is allowed to warm up to room temperature and stirred overnight. The reaction mixture is washed with 1 N HCl (20 mL). The organic layer is separated, dried over MgSO₄ and concentrated. The residue is triturated with heptane to afford 3-bromo-N-cyclohexyl-N-methyl-benzenesulfonamide as a solid (6 g). LCMS: R_{T}=3.54 minutes, MS: 332 (M+H).

Step 2. By proceeding in a similar manner to Example 10(a), method B, step 5, but substituting 1-(tert-butoxycarbonyl)indol-2-boronic acid (1.64 g) for 1-(tert-butoxycarbonyl)-5-methoxy-1H-indol-2-ylboronic acid and using 3-bromo-N-cyclohexyl-N-methyl-benzenesulfonamide (1.04 g), there is prepared 2-[3-(cyclohexyl-methyl-sulfamoyl)-phenyl]-indole-1-carboxylic acid tert-butyl ester as a solid (1.46 g). LCMS: R_{T}=3.69 minutes, MS: 469 (M+H).

Step 3. By proceeding in a similar manner to Example 10(a), method B, step 6, but substituting 2-[3-(cyclohexyl-methyl-sulfamoyl)-phenyl]-indole-1-carboxylic acid tert-butyl ester (1.45 g) for 2-(4-chloro-3-cyclohexylsulfamoyl-phenyl)-5-methoxy-indole-1-carboxylic acid tert-butyl ester, there is prepared N-cyclohexyl-3-(1H-indol-2-yl)-N-methyl benzenesulfonamide as a solid (1.06 g). LCMS: R_{T}=3.25 minutes, MS: 369 (M+H).

Step 4. By proceeding in a similar manner to Example 10(a), method B, step 7, but substituting N-cyclohexyl-3-(1H-indol-2-yl)-N-methyl benzenesulfonamide (1.06 g) for 2-chloro-N-cyclohexyl-5-(5-methoxy-1H-indol-2-yl)-benzenesulfonamide, there is prepared {2-[3-(cyclohexyl-methyl-sulfamoyl)-phenyl]-1H-indol-3-yl }-oxo-acetic acid methyl ester as a solid (910 mg). LCMS: R_{T}=3.35 minutes, MS: 455 (M+H).

Step 5. By proceeding in a similar manner to Example 10(a), method B, step 8, but substituting {2-[3-(cyclohexyl-methyl-sulfamoyl)-phenyl]-1H-indol-3-yl}-oxo-acetic acid methyl ester (300 mg) for [2-(4-chloro-3-cyclohexylsulfamoyl-phenyl)-5-methoxy-1H-indol-3-yl]-oxo-acetic acid methyl ester, there is prepared {2-[3-(cyclohexyl-methyl-sulfamoyl)-phenyl]-1H-indol-3-yl}-acetic acid methyl ester as a solid (190 mg). LCMS: R_{T}=3.59 minutes, MS: 441 (M+H).

Step 6. By proceeding in a similar manner to Example 10(a), method B, step 9, but substituting {2-[3-(cyclohexyl-methyl-sulfamoyl)-phenyl]-1H-indol-3-yl}-acetic acid methyl ester (157 mg) for [2-(4-chloro-3-cyclohexylsulfamoyl-phenyl)-5-methoxy-1H-indol-3-yl]-acetic acid methyl ester, there is prepared {2-[3-(cyclohexyl-methyl-sulfamoyl)-phenyl]-1H-indol-3-yl }-acetic acid as a solid (147 mg). LCMS: R_{T}=2.74 minutes, MS: 427 (M+H); ¹H NMR (300 MHz, CDCl₃) δ 1.23-1.4 (m, 5H), 1.52-1.62 (m, 3H), 1.72-1.75 (m, 2H), 2.8 (s, 3H), 3.78-3.85 (m, 1H), 3.88 (s, 2H), 7.18-7.31 (m, 2H), 7.42 (d, J=8.1 Hz, 1H), 7.6-7.71 (m, 2H), 7.82-7.89 (m, 2H), 8.09 (m, 1H), 8.35 (brs, 1H). IC₅₀=346 nM

### (f) [2-(3-Cyclohexylsulfamoyl-phenyl)-1H-indol-3-yl]-acetic acid

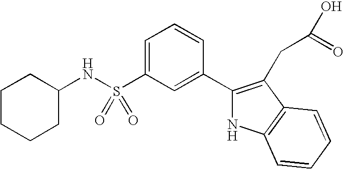

Step 1. 3-Bromo-benzenesulfonyl chloride (5 g) is slowly added to a solution of cyclohexylamine (3.4 mL) and DIEA (6.6 mL) in DCM (100 mL) at 0° C. The resulting mixture is warmed to room temperature and stirred for 20 hours. The reaction mixture is acidified with 2 N aqueous HCl (˜50 mL). The organic layer is separated, washed with water, brine, dried over sodium sulfate and evaporated in vacuo to afford 3-bromo-N-cyclohexyl-benzenesulfonamide as a solid (5.1 g). LCMS: R_{T}=2.94 minutes, MS: 318 (M+H).

Step 2. By proceeding in a similar manner to Example 10(a), method B, step 5, but substituting 1-(tert-butoxycarbonyl)indol-2-boronic acid (1.64 g) for 1-(tert-butoxycarbonyl)-5-methoxy-1H-indol-2-ylboronic acid and using 3-bromo-N-cyclohexyl-benzenesulfonamide (1 g), there is prepared 2-(3-cyclohexylsulfamoyl-phenyl)-indole-1-carboxylic acid tert-butyl ester as a white solid (1.13 g).

Step 3. By proceeding in a similar manner to Example 10(a), method B, step 6, but substituting 2-(3-cyclohexylsulfamoyl-phenyl)-indole-1-carboxylic acid tert-butyl ester (1.06 g) for 2-(4-chloro-3-cyclohexylsulfamoyl-phenyl)-5-methoxy-indole-1-carboxylic acid tert-butyl ester, there is prepared N-cyclohexyl-3-(1H-indol-2-yl)-benzenesulfonamide as a solid (700 mg). LCMS: R_{T}=3.45 minutes, MS: 355 (M+H);

Step 4. By proceeding in a similar manner to Example 10(a), method B, step 7, but substituting N-cyclohexyl-3-(1H-indol-2-yl)-benzenesulfonamide (700 mg) for 2-chloro-N-cyclohexyl-5-(5-methoxy-1H-indol-2-yl)-benzenesulfonamide, there is prepared [2-(3-cyclohexylsulfamoyl-phenyl)-1H-indol-3-yl]-oxo-acetic acid methyl ester as a solid (730 mg).

Step 5. By proceeding in a similar manner to Example 10(a), method B, step 8, but substituting [2-(3-cyclohexylsulfamoyl-phenyl)-1H-indol-3-yl]-oxo-acetic acid methyl ester (700 mg) for [2-(4-chloro-3-cyclohexylsulfamoyl-phenyl)-5-methoxy-1H-indol-3-yl]-oxo-acetic acid methyl ester, there is prepared [2-(3-cyclohexylsulfamoyl-phenyl)-1H-indol-3-yl]-acetic acid methyl ester as a solid (550 mg). LCMS: R_{T}=3.32 minutes, MS: 427 (M+H).

Step 6. By proceeding in a similar manner to Example 10(a), method B, step 9, but substituting [2-(3-cyclohexylsulfamoyl-phenyl)-1H-indol-3-yl]-acetic acid methyl ester (120 mg) for [2-(4-chloro-3-cyclohexylsulfamoyl-phenyl)-5-methoxy-1H-indol-3-yl]-acetic acid methyl ester, there is prepared [2-(3-cyclohexylsulfamoyl-phenyl)-1H-indol-3-yl]-acetic acid as a solid (105 mg). LCMS: R_{T}=2.54 minutes, MS: 413 (M+H). ¹H NMR (300 MHz, CD₃OD) δ 1.13-1.27 (m, 5H), 1.51-1.75 (m, 5H), 3.13 (m, 1H), 3.85 (s, 2H), 7.06 (t, J=7.5 Hz, 1H), 7.16 (t, J=7.5 Hz, 1H), 7.4 (d, J=8.1 Hz, 1H), 7.6 (d, J=7.8 Hz, 1H), 7.66 (t, J=7.5 Hz, 1H), 7.85 (d, J=7.8 Hz, 1H), 7.91 (d, J=7.8 Hz, 1H), 8.21 (s, 1H), 10.92 (brs, 1H). IC₅₀=106 nM

### (g) 2-[2-(3-Cyclohexylsulfamoyl-phenyl)-1H-indol-3-yl]-propionic acid

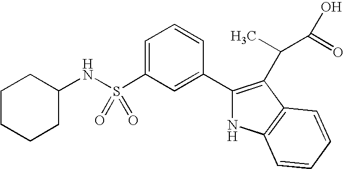

Step 1. Di-tert-butyl dicarbonate (450 mg) is added to a solution of [2-(3-cyclohexylsulfamoyl-phenyl)-1H-indol-3-yl]-acetic acid methyl ester (400 mg) triethylamine (0.3 mL) and 4-(dimethylamino)pyridine (23 mg) in DCM (5 mL). The reaction is stirred at room temperature for 1.5 hr. The reaction mixture is washed with 1 HCl (5 mL) and 1 N NaHCO₃ (5 mL). The organic layer is separated, dried over MgSO₄ and concentrated to afford 2-[3-(N-tert-butyloxycarbonyl)-cyclohexylsulfamoyl-phenyl]-3-methoxycarbonylmethyl-indole-1-carboxylic acid tert-butyl ester (600 mg). LCMS: R_{T}=3.32 minutes, MS: 427 (M+H).

Step 2. To a solution of 2-[3-(N-tert-butyloxycarbonyl)-cyclohexylsulfamoyl-phenyl]-3-methoxycarbonylmethyl-indole-1-carboxylic acid tert-butyl ester (590 mg) in DMF (5 mL) is added NaH (113 mg) in portion at 0° C. The resulting mixture is stirred at 0° C. for 15 minutes and MeI is added at 0° C. The reaction mixture is allowed to warm up to room temperature and stirred for 3 hr. The reaction is quenched by adding saturated NH₄Cl (10 mL). The mixture is extracted with EtOAc (20 mL). The organic layer is washed with water (3×10 mL), dried over MgSO₄ and concentrated. The residue is purified by flash chromatography on silica gel eluting with 10% to 45% EtOAc in heptane to afford 2-[3-(N-tert-butyloxycarbonyl)-cyclohexylsulfamoyl-phenyl]-3-(1-methoxycarbonyl-ethyl)-indole-1-carboxylic acid tert-butyl ester as a solid (445 mg). LCMS: R_{T}=3.82 minutes, MS: 649 (M+Na).

Step 3. TFA (1-mL) is added to a solution of 2-[3-(N-tert-butyloxycarbonyl)-cyclohexylsulfamoyl-phenyl]-3-(1-methoxycarbonyl-ethyl)-indole-1-carboxylic acid tert-butyl ester (100 mg) in DCM (6 mL). The reaction mixture is stirred at room temperature overnight. The mixture is concentrated in vacuo. The residue is dissolved in EtOAc and washed with 1 N NaHCO₃. The organic layer is separated, dried over MgSO₄ and concentrated. The residue is purified by flash chromatography on silica gel eluting with 10% to 50% EtOAc in heptane to afford 2-[2-(3-cyclohexylsulfamoyl-phenyl)-1H-indol-3-yl]-propionic acid methyl ester as a solid (65 mg). LCMS: R_{T}=3.94 minutes, MS: 663 (M+Na).

Step 4. By proceeding in a similar manner to Example 10(a), method B, step 9, but substituting 2-[2-(3-cyclohexylsulfamoyl-phenyl)-1H-indol-3-yl]-propionic acid methyl ester (65 mg) for [2-(4-chloro-3-cyclohexylsulfamoyl-phenyl)-5-methoxy-1H-indol-3-yl]-acetic acid methyl ester, there is prepared 2-[2-(3-cyclohexylsulfamoyl-phenyl)-1H-indol-3-yl]-propionic acid as a solid (41 mg). LCMS: R_{T}=3.02 minutes, MS: 427 (M+H); ¹H NMR (300 MHz, CDCl₃) δ 1.08-1.27 (m, 5H), 1.39-1.79 (m, 5H), 1.61 (s, 3H), 3.21 (m, 1H), 4.99 (t, J=8.4 Hz, 1H), 7.13 (t, J=6.9 Hz, 1H), 7.22 (t, J=5.7 Hz, 1H), 7.36 (d, J=8.1 Hz, 1H), 7.55 (t, J=7.8 Hz, 1H), 7.81 (t, J=8.4 Hz, 2H), 7.87 (d, J=8.1 Hz, 1H), 8.15 (s, 1H), 8.44 (brs, 1H).

### (h) [2-(4-Cyclohexylsulfamoyl-phenyl)-1H-indol-3-yl]-acetic acid

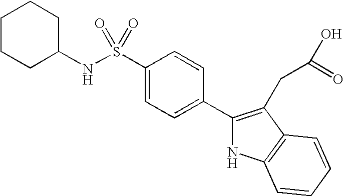

Step 1. 4-Bromo-benzenesulfonyl chloride (20 g) is slowly added to a solution of cyclohexylamine (14 mL) and DIEA (26 mL) in DCM (300 mL) at 0° C. The resulting mixture is warmed to room temperature and stirred for 20 hours. The reaction mixture is acidified with 2 N aqueous HCl (˜150 mL). The organic layer is separated, washed with water, brine, dried over sodium sulfate and evaporated in vacuo afford 4-bromo-N-cyclohexyl-benzenesulfonamide as a solid (19 g). LCMS: R_{T}=2.94 minutes, MS: 318 (M+H).

Step 2. By proceeding in a similar manner to Example 10(a), method B, step 5, but substituting 1-(tert-butoxycarbonyl)indol-2-boronic acid (1.64 g) for 1-(tert-butoxycarbonyl)-5-methoxy-1H-indol-2-ylboronic acid and using 4-bromo-N-cyclohexyl-benzenesulfonamide (1 g), there is prepared 2-(4-cyclohexylsulfamoyl-phenyl)-indole-1-carboxylic acid tert-butyl ester as a solid (1.38 g). LCMS: R_{T}=3.97 minutes, MS: 455 (M+H).

Step 3. By proceeding in a similar manner to Example 10(a), method B, step 6, but substituting 2-(4-cyclohexylsulfamoyl-phenyl)-indole-1-carboxylic acid tert-butyl ester (1.38 g) for 2-(4-chloro-3-cyclohexylsulfamoyl-phenyl)-5-methoxy-indole-1-carboxylic acid tert-butyl ester, there is prepared N-cyclohexyl-4-(1H-indol-2-yl)-benzenesulfonamide as a solid (1.02 g).

Step 4. By proceeding in a similar manner to Example 10(a), method B, step 7, but substituting N-cyclohexyl-4-(1H-indol-2-yl)-benzenesulfonamide (1 g) for 2-chloro-N-cyclohexyl-5-(5-methoxy-1H-indol-2-yl)-benzenesulfonamide, there is prepared [2-(4-cyclohexylsulfamoyl-phenyl)-1H-indol-3-yl]-oxo-acetic acid methyl ester as a solid (121 mg).

Step 5. By proceeding in a similar manner to Example 10(a), method B, step 8, but substituting [2-(4-cyclohexylsulfamoyl-phenyl)-1H-indol-3-yl]-oxo-acetic acid methyl ester (121 mg) for [2-(4-chloro-3-cyclohexylsulfamoyl-phenyl)-5-methoxy-1H-indol-3-yl]-oxo-acetic acid methyl ester, there is prepared [2-(4-cyclohexylsulfamoyl-phenyl)-1H-indol-3-yl]-acetic acid methyl ester as a solid (102 mg).

Step 6. By proceeding in a similar manner to Example 10(a), method B, step 9, but substituting [2-(4-cyclohexylsulfamoyl-phenyl)-1H-indol-3-yl]-acetic acid methyl ester (120 mg) for [2-(4-chloro-3-cyclohexylsulfamoyl-phenyl)-5-methoxy-1H-indol-3-yl]-acetic acid methyl ester, there is prepared [2-(4-cyclohexylsulfamoyl-phenyl)-1H-indol-3-yl]-acetic acid as a solid (60 mg). LCMS: R_{T}=2.5 minutes, MS: 413 (M+H); ¹H NMR (300 MHz, CD₃OD) δ 1.17-1.27 (m, 5H), 1.51-1.74 (m, 5H), 3.07 (m, 1H), 3.85 (s, 2H), 7.04 (t, J=6.9 Hz, 1H), 7.16 (t, J=7.2 Hz, 1H), 7.39 (d, J=7.8 Hz, 1H), 7.61 (d, J=7.8 Hz, 1H), 7.87 (d, J=8.4 Hz, 2H), 7.95 (d, J=8.4 Hz, 2H), 10.86 (s, 1H). IC₅₀=1162 nM

### (i) [2-(3-Cyclohexylsulfamoyl-4-methoxy-phenyl)-1H-indol-3-yl]-acetic acid

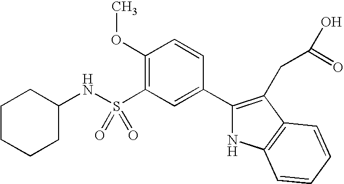

Step 1. 5-Bromo-2-methoxy-benzenesulfonyl chloride (10 g) is slowly added to a solution of cyclohexylamine (6 mL) and DIEA (12 mL) in DCM (200 mL) at 0° C. The resulting mixture is warned to room temperature and stirred for 20 hours. The reaction mixture is acidified with 2 N aqueous HCl (˜100 mL). The organic layer is separated, washed with water, brine, dried over sodium sulfate and evaporated in vacuo afford 5-bromo-N-cyclohexyl-2-methoxy-benzenesulfonamide as a solid (9.8 g). LCMS: R_{T}=2.84 minutes, MS: 348 (M+H).

Step 2. By proceeding in a similar manner to Example 10(a), method B, step 5, but substituting 1-(tert-butoxycarbonyl)indol-2-boronic acid (1.64 g) for 1-(tert-butoxycarbonyl)-5-methoxy-1H-indol-2-ylboronic acid and using 5-bromo-N-cyclohexyl-2-methoxy-benzenesulfonamide (1.09 g), there is prepared 2-(3-cyclohexylsulfamoyl-4-methoxy-phenyl)-indole-1-carboxylic acid tert-butyl ester as a solid (1.48 g). LCMS: R_{T}=3.99 minutes, MS: 485 (M+H).

Step 3. By proceeding in a similar manner to Example 10(a), method B, step 6, but substituting 2-(3-cyclohexylsulfamoyl-4-methoxy-phenyl)-indole-1-carboxylic acid tert-butyl ester (1.48 g) for 2-(4-chloro-3-cyclohexylsulfamoyl-phenyl)-5-methoxy-indole-1-carboxylic acid tert-butyl ester, there is prepared N-cyclohexyl-5-(1H-indol-2-yl)-2-methoxy-benzenesulfonamide as a solid (1.17 g).

Step 4. By proceeding in a similar manner to Example 10(a), method B, step 7, but substituting N-cyclohexyl-5-(1H-indol-2-yl)-2-methoxy-benzenesulfonamide (500 mg) for 2-chloro-N-cyclohexyl-5-(5-methoxy-1H-indol-2-yl)-benzenesulfonamide, there is prepared [2-(3-cyclohexylsulfamoyl-4-methoxy-phenyl)-1H-indol-3-yl]-oxo-acetic acid methyl ester as a solid (413 mg).

Step 5. By proceeding in a similar manner to Example 10(a), method B, step 8, but substituting [2-(3-cyclohexylsulfamoyl-4-methoxy-phenyl)-1H-indol-3-yl]-oxo-acetic acid methyl ester (310 mg) for [2-(4-chloro-3-cyclohexylsulfamoyl-phenyl)-5-methoxy-1H-indol-3-yl]-oxo-acetic acid methyl ester, there is prepared [2-(3-cyclohexylsulfamoyl-4-methoxy-phenyl)-1H-indol-3-yl]-acetic acid methyl ester as a solid (312 mg).

Step 6. By proceeding in a similar manner to Example 10(a), method B, step 9, but substituting [2-(3-cyclohexylsulfamoyl-4-methoxy-phenyl)-1H-indol-3-yl]-acetic acid methyl ester (312 mg) for [2-(4-chloro-3-cyclohexylsulfamoyl-phenyl)-5-methoxy-1H-indol-3-yl]-acetic acid methyl ester, there is prepared [2-(3-cyclohexylsulfamoyl-4-methoxy-phenyl)-1H-indol-3-yl]-acetic acid as a solid (19 mg). LCMS: R_{T}=2.54 minutes, MS: 443 (M+H); ¹H NMR (300 MHz, CD₃OD) δ 1.15-1.37 (m, 5H), 1.5-1.74 (m, 5H), 3.08 (m, 1H), 3.78 (brs, 2H), 4 (s, 3H), 7.03 (t, J=7.2 Hz, 1H), 7.12 (t, J=7.2 Hz, 1H), 7.28 (d, J=8.4 Hz, 1H), 7.36 (d, J=8.1 Hz, 1H), 7.56 (d, J=7.5 Hz, 1H), 7.93 (d, J=8.1 Hz, 1H), 8.17 (s, 1H). IC₅₀=63 nM

### (j) [2-(3-Chloro-4-cyclohexylsulfamoyl-phenyl)-1H-indol-3-yl]-acetic acid

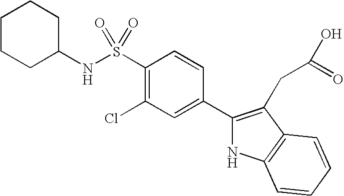

Step 1. By proceeding in a similar manner to Example 10(e), step 1, but substituting cyclohexylamine (2.06 g) for cyclohexyl-methyl-amine and using 4-bromo-2-chloro-benzenesulfonyl chloride (5.3 g), there is prepared 4-bromo-2-chloro-N-cyclohexyl-benzenesulfonamide (6.4 g). LCMS: R_{T}=3.02 minutes, MS: 352 (M+H).

Step 2. By proceeding in a similar manner to Example 10(a), method B, step 5, but substituting 1-(tert-butoxycarbonyl)indol-2-boronic acid (1.26 g) for 1-(tert-butoxycarbonyl)-5-methoxy-1H-indol-2-ylboronic acid and using 4-bromo-2-chloro-N-cyclohexyl-benzenesulfonamide (1 g), there is prepared 2-(3-chloro-4-cyclohexylsulfamoyl-phenyl)-indole-1-carboxylic acid tert-butyl ester as a solid (1.14 g),

Step 3. By proceeding in a similar manner to Example 10(a), method B, step 6, but substituting 2-(3-chloro-4-cyclohexylsulfamoyl-phenyl)-indole-1-carboxylic acid tert-butyl ester (1.14 g) for 2-(4-chloro-3-cyclohexylsulfamoyl-phenyl)-5-methoxy-indole-1-carboxylic acid tert-butyl ester, there is prepared 2-chloro-N-cyclohexyl-4-(1H-indol-2-yl)benzenesulfonamide as a solid (901 mg).

Step 4. By proceeding in a similar manner to Example 10(a), method B, step 7, but substituting 2-chloro-N-cyclohexyl-4-(1H-indol-2-yl)benzenesulfonamide (500 mg) for 2-chloro-N-cyclohexyl-5-(5-methoxy-1H-indol-2-yl)-benzenesulfonamide, there is prepared [2-(3-chloro-4-cyclohexylsulfamoyl-phenyl)-1H-indol-3-yl]-oxo-acetic acid methyl ester as a solid (600 mg).

Step 5. By proceeding in a similar manner to Example 10(a), method B, step 8, but substituting [2-(3-chloro-4-cyclohexylsulfamoyl-phenyl)-1H-indol-3-yl]-oxo-acetic acid methyl ester (500 mg) for [2-(4-chloro-3-cyclohexylsulfamoyl-phenyl)-5-methoxy-1H-indol-3-yl]-oxo-acetic acid methyl ester, there is [2-(3-chloro-4-cyclohexylsulfamoyl-phenyl)-1H-indol-3-yl]-acetic acid methyl ester as a solid (310 mg). LCMS: R_{T}=3.50 minutes, MS: 461 (M+H).

Step 6. By proceeding in a similar manner to Example 10(a), method B, step 9, but substituting [2-(3-chloro-4-cyclohexylsulfamoyl-phenyl)-1H-indol-3-yl]-acetic acid methyl ester (277 mg) for [2-(4-chloro-3-cyclohexylsulfamoyl-phenyl)-5-methoxy-1H-indol-3-yl]-acetic acid methyl ester, there is prepared [2-(3-chloro-4-cyclohexylsulfamoyl-phenyl)-1H-indol-3-yl]-acetic acid as a white solid (158 mg). LCMS: R_{T}=2.54 minutes, MS: 447 (M+H); ¹H NMR (300 MHz, CDCl₃) δ 1.15-1.37 (m, 5H), 1.5-1.79 (m, 5H), 3.19 (m, 1H), 3.88 (s, 2H), 5.1 (d, J=7.5 Hz, 1H), 7.19 (t, J=7.8 Hz, 1H), 7.28 (t, J=8.4 Hz, 1H), 7.43 (d, J=8.1 Hz, 1H), 7.68 (t, J=7.8 Hz, 2H), 7.80 (s, 1H), 8.12 (d, J=8.1 Hz, 1H), 8.60 (s, 1H). IC₅₀=1222 nM

### (k) [2-(3-Cyclohexylsulfamoyl-4-methyl-phenyl)-1H-indol-3-yl]-acetic acid

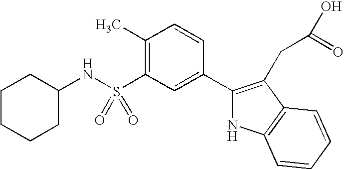

Step 1. Chlorosulfonic acid (7.3 mL) is slowly added to a solution of 4-bromotoluene (3 g) in DCM (29 mL) at 0° C. The resulting mixture is stirred at 0° C. for 4 hr, and poured onto crushed ice (500 mL). The mixture is extracted with DCM (250 mL). The organic layer is separated, dried over MgSO₄ and concentrated to afford 5-bromo-2-methyl-benzenesulfonyl chloride as an oil (2.65 g).

Step 2. By proceeding in a similar manner to Example 10(e), step 1, but substituting cyclohexylamine (1.17 g) for cyclohexyl-methyl-amine and using 5-bromo-2-methyl-benzenesulfonyl chloride (2.65 g), there is prepared 5-bromo-2-methyl-N-cyclohexyl-benzenesulfonamide as a crystal (2.7 g). LCMS: Rt=2.97 minutes, MS: 332 (M+H).

Step 3. By proceeding in a similar manner to Example 10(a), method B, step 5, but substituting 1-(tert-butoxycarbonyl)indol-2-boronic acid (668 mg) for 1-(tert-butoxycarbonyl)-5-methoxy-1H-indol-2-ylboronic acid and using 5-bromo-2-methyl-N-cyclohexyl-benzenesulfonamide (500 mg), there is prepared 2-(3-cyclohexylsulfamoyl-4-methyl-phenyl)-indole-1-carboxylic acid tert-butyl ester as a solid (361 mg).

Step 4. By proceeding in a similar manner to Example 10(a), method B, step 6, but substituting 2-(3-cyclohexylsulfamoyl-4-methyl-phenyl)-indole-1-carboxylic acid tert-butyl ester (360 mg) for 2-(4-chloro-3-cyclohexylsulfamoyl-phenyl)-5-methoxy-indole-1-carboxylic acid tert-butyl ester, there is prepared N-cyclohexyl-5-(1H-indol-2-yl)-2-methyl-benzenesulfonamide as a solid (280 mg).

Step 5. By proceeding in a similar manner to Example 10(a), method B, step 7, but substituting N-cyclohexyl-5-(1H-indol-2-yl)-2-methyl-benzenesulfonamide (280 mg) for 2-chloro-N-cyclohexyl-5-(5-methoxy-1H-indol-2-yl)-benzenesulfonamide, there is prepared [2-(3-cyclohexylsulfamoyl-4-methyl-phenyl)-1H-indol-3-yl]-oxo-acetic acid methyl ester as a solid (230 mg). LCMS: R_{T}=2.8 minutes, MS: 455 (M+H).

Step 6. By proceeding in a similar manner to Example 10(a), method B, step 8, but substituting [2-(3-cyclohexylsulfamoyl-4-methyl-phenyl)-1H-indol-3-yl]-oxo-acetic acid methyl ester (210 mg) for [2-(4-chloro-3-cyclohexylsulfamoyl-phenyl)-5-methoxy-1H-indol-3-yl]-oxo-acetic acid methyl ester, there is [2-(3-cyclohexylsulfamoyl-4-methyl-phenyl)-1H-indol-3-yl]-acetic acid methyl ester as a solid (162 mg). LCMS: Rₜ=3.3 minutes, MS: 441 (M+H); ¹H NMR (300 MHz, CDCl₃) δ 1.09-1.28 (m, 5H), 1.5-1.62 (m, 3H), 1.78-1.81 (m, 2H), 2.7 (s, 3H), 3.2 (m, 1H), 3.73 (s, 3H), 3.83 (s, 2H), 4.56 (d, J=7.8 Hz, 1H), 7.15-7.28 (m, 2H), 7.42 (t, J=7.2 Hz, 2H), 7.68 (d, J=7.5 Hz, 1H), 7.81 (dd, J=7.8, 1.8 Hz, 1H), 8.28 (d, J=2.1 Hz, 1H), 8.34 (s, 1H).

Step 7. By proceeding in a similar manner to Example 10(a), method B, step 9, but substituting [2-(3-cyclohexylsulfamoyl-4-methyl-phenyl)-1H-indol-3-yl]-acetic acid methyl ester (150 mg) for [2-(4-chloro-3-cyclohexylsulfamoyl-phenyl)-5-methoxy-1H-indol-3-yl]-acetic acid methyl ester, there is prepared [2-(3-cyclohexylsulfamoyl-4-methyl-phenyl)-1H-indol-3-yl]-acetic acid as a beige solid (133 mg). LCMS: Rₜ=2.94 minutes, MS: 427 (M+H); ¹H NMR (300 MHz, CD₃OD) δ 1.11-1.28 (m, 5H), 1.5-1.54 (m, 2H), 1.64-1.7 (m, 3H), 2.69 (brs, 3H), 3.08 (m, 1H), 3.84 (brs, 2H), 7.05 (t, J=7.8 Hz, 1H), 7.15 (t, J=8.4 Hz, 1H), 7.39 (d, J=8.1 Hz, 1H), 7.47 (d, J=7.8 Hz, 1H), 7.58 (d, J=7.5 Hz, 1H), 7.80 (d, J=7.5 Hz, 1H), 8.28 (s, 1H). IC₅₀=2 nM

### Example 11

### [2-(3-cyclohexylsulfamoyl-5-trifluoromethyl-phenyl)-1H-indol-3-yl]-acetic acid methyl ester

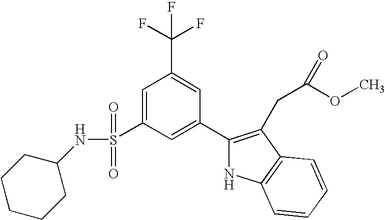

Step 1. 3-Bromo-5-(trifluoromethyl)benzenesulfonyl chloride (2 g) is dissolved in anhydrous acetonitrile (50 mL). Potassium carbonate (0.85 g) is added and the solution is cooled to 0° C. Cyclohexyl amine (0.61 g) is added dropwise at 0° C. as a solution in anhydrous acetonitrile (5 mL). The reaction mixture is allowed to warm to room temperature and stirred for 18 hours. The reaction mixture is filtered. The filtrate is evaporated under reduced pressure. The residue is partitioned between EtOAc and 10% aqueous HCl and the layers are separated. The organic layer is washed with saturated 10% NaHCO₃ solution and brine. The organic layer is dried (MgSO₄), filtered, and evaporated to dryness. The crude material is chromatographed on silica gel eluting with heptane, and 10% EtOAc/heptane. Product containing fractions are combined and evaporated under reduced pressure. The residue is triturated with heptane and the resulting solid is filtered, washed with heptane, and dried under vacuum to afford 3-bromo-N-cyclohexyl-5-trifluoromethyl-benzenesulfonamide (1.72 g). LCMS: R_{T}=3.09 minutes, MS: 384 (M−H).

Step 2. 3-Bromo-N-cyclohexyl-5-trifluoromethyl-benzenesulfonamide (0.5 g), 1-N-Bo-2-indoleboronic acid (0.67 g), and CsF (0.39 g) are suspended in 10:1 dioxane:water (22 mL). The solution is purged with N₂ and PdCl₂(dppf)₂ (105 mg) is added. The solution is heated to 80° C. for 5 hours. The mixture is evaporated under reduced pressure. The residue is treated with EtOAc/heptane, filtered and washed with heptane. The filtrate is evaporated under reduced pressure and the residue is chromatographed on silica gel eluting with 3-4% EtOAc/heptane to afford 2-(3-cyclohexylsulfamoyl-5-trifluoromethyl-phenyl)-indole-1-carboxylic acid tert-butyl ester (0.61 g) as a tan solid. LCMS: R_{T}=3.64 minutes; MS: 523 (M+H).

Step 3. 2-(3-Cyclohexylsulfamoyl-5-trifluoromethyl-phenyl)-indole-1-carboxylic acid tert-butyl ester (0.58 g) is dissolved in TFA (8 mL) and stirred at room temperature for 1 hour. The TFA is removed under reduced pressure and the residue is triturated with heptane. The resulting precipitate is filtered, washed and dried under vacuum. The crude material is partitioned between EtOAc and saturated NaHCO₃ and the layers are separated. The organic layer is washed with saturated NaHCO₃, water, and brine. The organic layer is dried (MgSO₄), filtered, and evaporated under reduced pressure. The material is recrystallized from DCM/heptane to afford N-cyclohexyl-3-(1H-indol-2-yl)-5-trifluoromethyl-benzenesulfonamide (0.35 g) as a solid. LCMS: R_{T}=3.29 minutes, MS: 423 (M+H).

Step 4. N-Cyclohexyl-3-(1H-indol-2-yl)-5-trifluoromethyl-benzenesulfonamide (0.3 g) is suspended in anhydrous Et₂O (25 mL). Oxalyl chloride (0.14 g) in Et₂O (1 mL) is added dropwise at room temperature and the mixture is stirred for 7 hours. MeOH (2 mL) is added and the reaction mixture is stirred for 10 minutes, and evaporated under reduced pressure. The residue is partitioned between EtOAc and saturated NaHCO₃ and the layers separated. The organic layer is washed with saturated NaHCO₃. The organic layer is dried (MgSO₄), filtered, and evaporated under reduced pressure. The crude material is chromatographed on silica gel eluting with 15% EtOAc/Heptane to afford [2-(3-cyclohexylsulfamoyl-5-trifluoromethyl-phenyl)-1H-indol-3-yl]-oxo-acetic acid methyl ester (0.35 g) as a solid. LCMS: R_{T}=3.19 minutes, MS: 509 (M+H).

Step 5. [2-(3-Cyclohexylsulfamoyl-5-trifluoromethyl-phenyl)-1H-indol-3-yl]-oxo-acetic acid methyl ester (0.32 g) is dissolved in TFA (6 mL). Triethylsilane (0.15 g) is added and the solution is stirred at room temperature for 7 hours. The reaction mixture is evaporated and the residue is dissolved in EtOAc. The organic layer is washed with saturated NaHCO₃, water, and brine. The organic layer is dried (MgSO₄), filtered, and evaporated under reduced pressure. The crude material is chromatographed on silica gel eluting with 15% EtOAc/heptane. The material is recrystallized from DCM/heptane to afford [2-(3-cyclohexylsulfamoyl-5-trifluoromethyl-phenyl)-1H-indol-3-yl]-acetic acid methyl ester (0.17 g) as a solid. LCMS: R_{T}=4.27 minutes, MS: 495 (M+H); ¹H NMR (300 MHz, CDCl₃) δ 1.08-1.83 (m, 10H), 3.28 (m, 1H), 3.74 (s, 3H), 3.82 (s, 2H), 4.77 (d, 1H, J=7.7 Hz), 7.19-7.3 (m, 2H), 7.42 (d, 1H, J=8.3 Hz), 7.73 (d, 1H, J=7.9 Hz), 8.13 (s, 1H), 8.18 (s, 1H), 8.44 (m, 2H).

### Example 12

### [2-(3-cyclohexylsulfamoyl-5-trifluoromethyl-phenyl)-1H-indol-3-yl]-acetic acid

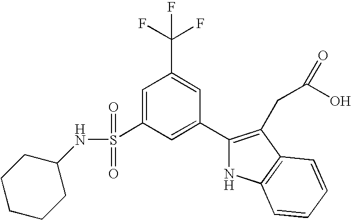

[2-(3-Cyclohexylsulfamoyl-5-trifluoromethyl-phenyl)-1H-indol-3-yl]-acetic acid methyl ester (144 mg, see Example 11) is suspended in 1:1 MeOH:H₂O (6 mL). Lithium hydroxide monohydrate (24 mg) is added and the suspension is heated to 80° C. for 4 hours, and stirred at room temperature overnight. The solvent is removed under reduced pressure. The residue is partitioned between EtOAc and 10% aqueous HCl and the layers separated. The organic layer is washed with additional 10% HCl and brine, dried (MgSO₄), filtered, and evaporated. The residue is recrystallized from EtOAc/heptane to afford [2-(3-cyclohexylsulfamoyl-5-trifluoromethyl-phenyl)-1H-indol-3-yl]-acetic acid (89 mg). LCMS: R_{T}=2.59 minutes, MS: 481 (M+H); (¹HNMR, CD₃OD) δ 1.1-1.78 (m, 10H), 3.19 (m, 1H), 3.87 (s, 2H), 7.10 (t, 1H, J=7.7 Hz), 7.21 (t, 1H, J=7.2 Hz), 7.43 (d, 1H, J=8 Hz), 7.64 (d, 1H, J=7.9 Hz), 8.11 (s, 1H), 8.26 (s, 1H), 8.47 (s, 1H), 11.16 (s, 1H). IC₅₀₌₂₃₂ nM

### Example 13

### [2-(3-benzenesulfonylamino-4-chlorophenyl)-1H-indol-3-yl]-acetic acid

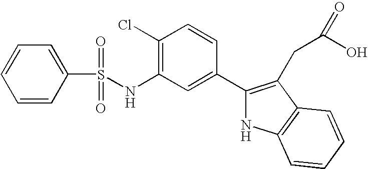

Step 1. 5-Bromo-2-chloro-phenylamine (0.48 g) is dissolved in pyridine (6 mL) and the solution is cooled to 0° C. Benzenesulfonyl chloride (0.41 g) in DCM (2 mL) is added dropwise. The solution is stirred at 0° C. for 30 minutes and at room temperature for 2 hours. Pyridine is removed under reduced pressure and the residue is dissolved in EtOAc. The organic layer is washed with 10% aqueous HCl, saturated NaHCO₃, and brine. The organic layer is dried (MgSO₄), filtered and evaporated and the crude material is recrystallized from EtOAc/heptane to afford N-(5-bromo-2-chloro-phenyl)-benzenesulfonamide (0.62 g) as a solid. LCMS: R_{T}=3.06 minutes, MS: 346 (M+H).

Step 2. N-(5-Bromo-2-chloro-phenyl)-benzenesulfonamide (0.61 g), 1-N-boc-2-indole boronic acid (0.92 g), and CsF (0.54 g) are suspended in 10:1 dioxane:water (22 mL) and the solution is purged with N₂. PdCl₂(dppf)₂ (145 mg) is added and the mixture is heated to 80° C. for 3 hours. The reaction mixture is concentrated under reduced pressure and the residue is passed through a plug of silica gel eluting with EtOAc. The EtOAc filtrate is evaporated to dryness and the residue is treated with EtOAc/heptane. The precipitate is filtered, washed with heptane and dried. The material is purified by chromatography on silica gel eluting with heptane and 4-20% EtOAc/heptane to afford 2-(3-benzenesulfonylamino-4-chloro-phenyl)-indole-1-carboxylic acid tert-butyl ester as a solid (0.72 g). LCMS: R_{T}=3.39 minutes, MS: 483 (M+H).

Step 3. 2-(3-Benzenesulfonylamino-4-chloro-phenyl)-indole-1-carboxylic acid tert-butyl ester (0.6 g) is dissolved in TFA (6 mL) and stirred at room temperature for 1 hour. The TFA is removed under reduced pressure and the residue is dissolved in EtOAc. The solution is washed with saturated aqueous NaHCO₃ solution, water, and brine, dried (MgSO₄), filtered, and concentrated. The residue is chromatographed on silica gel eluting with DCM. The product containing fractions are evaporated. The resulting residue is recrystallized from EtOAc/heptane to afford N-[2-chloro-5-(1H-indol-2-yl)-phenyl]-benzenesulfonamide as a solid (430 mg). LCMS: R_{T}=2.94 minutes, MS: 383 (M+H).

Step 4. N-[2-Chloro-5-(1H-indol-2-yl)-phenyl]-benzenesulfonamide (0.4 g) is suspended in anhydrous Et₂O (25 mL) and oxalyl chloride (0.2 g) is added dropwise at room temperature. The resulting suspension is stirred for 10 hours. MeOH (5 mL) is added and the solution is stirred 10 minutes. The mixture is concentrated under reduced pressure. The residue is recrystallized from DCM/heptane to afford [2-(3-benzenesulfonylamino-4-chlorophenyl)-1H-indole-3-yl]-oxo-acetic acid methyl ester (379 mg) as a powder. LCMS: R_{T}=2.65 minutes, MS: 469 (M+H).

Step 5. [2-(3-Benzenesulfonylamino-4-chlorophenyl)-1H-indole-3-yl]-oxo-acetic acid methyl ester (120 mg) is dissolved in TFA (2 mL). Triethylsilane (59 mg) is added and the solution is stirred at room temperature for 6 hours. The reaction mixture is concentrated and the residue is dissolved in EtOAc and washed with saturated aqueous NaHCO₃ solution. The organic layer is dried (MgSO₄), filtered, and evaporated under reduced pressure. The crude material is chromatographed on silica gel eluting with 10-15% EtOAc/heptane to afford [2-(3-benzenesulfonylamino-4-chlorophenyl)-1H-indol-3-yl]-acetic acid methyl ester as a solid (41 mg). LCMS: R_{T}=2.92 minutes, MS: 455 (M+H).

Step 6. [2-(3-Benzenesulfonylamino-4-chlorophenyl)-1H-indol-3-yl]-acetic acid methyl ester (40 mg) is dissolved in 1:1 MeOH:water (2 mL). Lithium hydroxide monohydrate (7.4 mg) is added and the mixture is heated to 80° C. for 6 hours. MeOH is removed under reduced pressure and the residue is partitioned between EtOAc and 10% aqueous HCl. The EtOAc layer is washed with 10% aqueous HCl, dried (MgSO₄), filtered, and concentrated. The residue is treated with Et₂O/heptane to afford [2-(3-benzenesulfonylamino-4-chlorophenyl)-1H-indol-3-yl]-acetic acid as a solid (38 mg). LCMS: R_{T}=2.47 minutes, MS: 441 (M+H); ¹H NMR (300 MHz, CD₃OD) δ 3.83 (s, 2H), 7.05 (t, 1H, J=7.5 Hz), 7.15 (t, 1H, J=7 Hz), 7.47 (m, 7H), 7.78 (d, 2H, J=7.3 Hz), 7.9 (d, 1H, J=2.1 Hz). IC₅₀=39 nM

### Example 14

### {2-[4-chloro-3-(cyclohexanecarbonyl-amino)-phenyl]-1H-indol-3-yl}-acetic acid

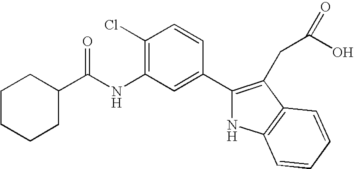

Step 1. By proceeding in a manner similar to Example 13, step 1, but substituting cyclohexane carbonyl chloride (0.5 g) for benzenesulfonyl chloride, there is prepared cyclohexanecarboxylic acid (5-bromo-2-chloro-phenyl)-amide (420 mg) as a powder. LCMS: R_{T}=3.51 minutes, MS: 316 (M+H).

Step 2. By proceeding in a manner similar to Example 13, step 2, but substituting cyclohexanecarboxylic acid (5-bromo-2-chloro-phenyl)-amide (400 mg) for N-(5-bromo-2-chloro-phenyl)-benzenesulfonamide, there is prepared 2-[4-chloro-3-(cyclohexanecarbonylamino)-phenyl]-indole-1-carboxylic acid tert-butyl ester (410 mg) as an oil. LCMS: R_{T}=3.74 minutes, MS: 453 (M+H).

Step 3. By proceeding in a manner similar to Example 13, step 3, but substituting 2-[4-chloro-3-(cyclohexanecarbonylamino)-phenyl]-indole-1-carboxylic acid tert-butyl ester (400 mg) for 2-(3-benzenesulfonylamino-4-chloro-phenyl)-indole-1-carboxylic acid tert-butyl ester, there is prepared cyclohexanecarboxylic acid [2-chloro-5-(1H-indol-2-yl)-phenyl]-amide (230 mg). LCMS: R_{T}=3.57 minutes, MS: 353 (M+H).

Step 4. By proceeding in a manner similar to Example 13, step 4, but substituting cyclohexanecarboxylic acid [2-chloro-5-(1H-indol-2-yl)-phenyl]-amide (200 mg) for N-[2-chloro-5-(1H-indol-2-yl)-phenyl]-benzenesulfonamide, there is prepared {2-[4-chloro-3-(cyclohexanecarbonyl-amino)-phenyl]-1H-indol-3-yl}-oxo-acetic acid methyl ester (200 mg).

Step 5. By proceeding in a manner similar to Example 13, step 5, but substituting {2-[4-chloro-3-(cyclohexanecarbonyl-amino)-phenyl]-1H-indol-3-yl}-oxo-acetic acid methyl ester (180 mg) for [2-(3-benzenesulfonylamino-4-chlorophenyl)-1H-indole-3-yl]-oxo-acetic acid methyl ester, there is prepared {2-[4-chloro-3-(cyclohexanecarbonyl-amino)-phenyl]-1H-indol-3-yl}-acetic acid methyl ester (156 mg). LCMS: R_{T}=3.12 minutes, MS: 425 (M+H).

Step 6. By proceeding in a manner similar to Example 13, step 6, but substituting {2-[4-chloro-3-(cyclohexanecarbonyl-amino)-phenyl]-1H-indol-3-yl}-acetic acid methyl ester (150 mg) for [2-(3-benzenesulfonylamino-4-chlorophenyl)-1H-indol-3-yl]-acetic acid methyl ester, there is prepared {2-[4-chloro-3-(cyclohexanecarbonyl-amino)-phenyl]-1H-indol-3-yl}acetic acid (35 mg). LCMS: R_{T}=2.86 minutes, MS: 411 (M+H); ¹H NMR (300 MHz, CD₃OD) δ 1.27-1.99 (m, 10H), 2.51 (m, 1H), 3.84 (s, 2H), 7.04 (t, 1H, J=7.2 Hz), 7.14 (t, 1H, J=6.9 Hz), 7.37 (d, 1H, J=8 Hz), 7.57 (m, 3H), 7.99 (s, 1H), 10.81 (s, 1H). IC₅₀=5856 nM

### Example 15

### 2-(4-Chloro-3-cyclohexylsulfamoyl-phenyl)-1H-indole-3-carboxylic acid

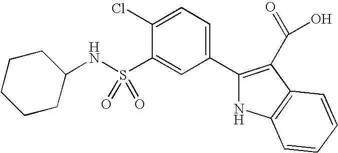

Step 1: To a solution of 2-chloro-N-cyclohexyl-5-(1H-indol-2-yl)-benzenesulfonamide (500 mg) in 1,2-dichloroethane (20 mL) is added anhydrous DMF (145 mg) followed by phosphorus oxychloride (364 mg). The reaction mixture is heated at 90° C. for 6 hrs and is allowed to cool down to room temperature. The mixture is diluted with ice-water (10 mL) and stirred for 1 hr with a 1 M aqueous solution of sodium acetate (5 mL). The mixture is extracted with DCM, washed with water, brine, dried over sodium sulfate and concentrated. The crude is purified by preparative HPLC separation (mobile phase: acetonitrile-water with 0.1% TFA; gradient 10-100% over 10 minutes), to afford 2-chloro-N-cyclohexyl-5-(3-formyl-1H-indol-2-yl)-benzenesulfonamide (350 mg). LCMS: R_{T}=2.83 minutes, MS: 417 (M+H).). ¹H NMR (300 MHz, DMSO-D₆) δ0.8-1.8 (m, 10H), 3.08 (m, 1H), 7.3 (m, 2H), 7.55 (d, J=7.5 Hz, 1H), 7.88 (d, J=8.3 Hz, 1H), 8.05 (m, 2H), 8.22 (d, J=7.2 Hz, 1H), 8.32 (d, J=2.2 Hz, 1H), 9.98 (s, 1H), 12.65 (s, 1H).

Step 2: To a solution of 2-chloro-N-cyclohexyl-5-(3-formyl-1H-indol-2-yl)-benzenesulfonamide (200 mg) in 1,4-dioxane (10 mL) and water (5 mL) is added anhydrous sodium chlorite (75 mg) followed by sulfamic acid (350 mg). The reaction mixture is stirred for 1 hour. Aqueous saturated sodium bicarbonate solution (3 mL) is added slowly and stirred for 10 minutes. The mixture is concentrated. The residue is diluted with EtOAc (50 mL), washed with 2N aqueous HCl (25 mL), water, dried over sodium sulfate and concentrated. The crude is purified by preparative HPLC separation (mobile phase: acetonitrile-water with 0.1% TFA; gradient 10-100% over 10 minutes) to afford 2-(4-chloro-3-cyclohexylsulfamoyl-phenyl)-1H-indole-3-carboxylic acid (5 mg). LCMS: R_{T}=2.6 minutes, MS: 433 (M+H). ¹H NMR (300 MHz, DMSO-D₆) δ 0.8-1.8 (m, 10H), 3.06 (m, 1H), 7.22 (m, 2H), 7.47 (d, J=7 Hz, 1H), 7.77 (d, J=8.3 Hz, 1H), 7.92 (m, 2H), 8.09 (d, J=7 Hz, 1H), 8.3 (d, J=2.2 Hz, 1H), 12.15 (broad s, 1H), 12.25 (s, 1H). IC₅₀=741 nM

### Example 16

### 2-(4-Chloro-3-cyclohexylsulfamoyl-phenyl)-1H-indole-6-carboxylic acid

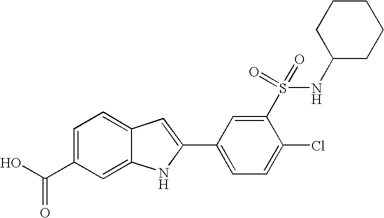

Step 1. By proceeding in a similar manner to Example 10(a), method B, step 5, but substituting 1-(tert-butyloxycarbonyl)-6-methoxycarbonyl-indol-2-yl boronic acid (150 mg) for 1-(tert-butoxycarbonyl)-5-methoxy-1H-indol-2-ylboronic acid and using 5-bromo-2-chloro-N-cyclohexyl-benzenesulfonamide (128 mg), there is prepared 2-(4-chloro-3-cyclohexylsulfamoyl-phenyl)-indole-1,6-dicarboxylic acid 1-tert-butyl ester 6-methyl ester as a solid (90 mg).

Step 2. By proceeding in a similar manner to Example 10(a), method B, step 6, but substituting 2-(4-chloro-3-cyclohexylsulfamoyl-phenyl)-indole-1,6-dicarboxylic acid 1-tert-butyl ester 6-methyl ester (90 mg) for 2-(4-chloro-3-cyclohexylsulfamoyl-phenyl)-5-methoxy-indole-1-carboxylic acid tert-butyl ester, there is prepared 2-(4-chloro-3-cyclohexylsulfamoyl-phenyl)-1H-indole-6-carboxylic acid methyl ester as a solid (69 mg).

Step 3. By proceeding in a similar manner to Example 10(a), method B, step 9, but substituting 2-(4-chloro-3-cyclohexylsulfamoyl-phenyl)-1H-indole-6-carboxylic acid methyl ester (64 mg) for [2-(4-chloro-3-cyclohexylsulfamoyl-phenyl)-5-methoxy-1H-indol-3-yl]-acetic acid methyl ester, there is prepared 2-(4-chloro-3-cyclohexylsulfamoyl-phenyl)-1H-indole-6-carboxylic acid as a solid (45 mg). LCMS: R_{T}=4.75 minutes, MS: 433.11 (M+H); ¹H NMR (300 MHz, DMSO-D₆) δ 1.1-1.63 (m, 10H), 3.07 (m, 1H), 7.15 (s, 1H), 7.67 (s, 2H), 7.79 (m, 1H), 8.02-8.14 (m, 3H), 8.50 (s, 1H), 12.20 (s, 1H), 12.61 (s, 1H). IC₅₀=510 nM

### Pharmacological Testing

The inhibitory effects of the compounds according to the invention are assessed in a human DP functional assay. A cAMP assay is employed using the human cell line LS174T, which expresses the endogenous DP receptor. The protocol is similar to that described previously (Wright D H, Ford-Hutchinson A W, Chadee K, Metters K M, The human prostanoid DP receptor stimulates mucin secretion in LS174T cells, Br J Pharmacol. 131(8):1537-45 (2000)).

Protocol for SPA cAMP Assay in Human LS174 T Cells

Materials

PGD2 (Cayman Chemical Cat#12010)
IBMX (Sigma Cat# 5879)
cAMP SPA direct screening assay system (Amersham code RPA 559)
96-well cell plates (Wallac Cat# 1450-516)
Wallac 1450 Microplate Trilux scintillation counter (PerkinElmer)
Plate sealers
Eppendorf tubes
Dulbecco's Phosphate-Buffered Saline (PBS) (Invitrogen Cat#14040-133)
Distilled water
Vortex
Magnetic stirrer and stirrer bars
   Reagent Preparation:

All reagents should be allowed to equilibrate to room temperature before reconstitution.

1× Assay Buffer

Transfer the contents of the bottle to a 500 mL graduated cylinder by repeated washing with distilled water. Adjust the final volume to 500 mL with distilled water and mix thoroughly.

Lysis Reagent 1 & 2

Dissolve each of the lysis reagents 1 and 2 in 200 mL assay buffer respectively. Leave at room temperature for 20 minutes to dissolve.

SPA Anti-Rabbit Beads

Add 30 mL of lysis buffer 2 to the bottle. Gently shake the bottle for 5 minutes.

Antiserum

Add 15 mL of lysis buffer 2 to each vial, and gently mix until the contents are completely dissolved.

Tracer (I¹²⁵-cAMP)

Add 14 mL lysis buffer 2 to each vial and gently mix until the contents are completely dissolved.

Preparation of Immunoreagent

1) Add equal volumes of tracer, antiserum and SPA anti-rabbit reagent to a bottle, ensuring that a sufficient volume of this mixture is prepared for the desired number of wells (150 μL/well).
2) Mix thoroughly.
3) This immunoreagent solution should be freshly prepared before each assay and not re-used.
   Standard
1) Add 1 mL lysis buffer 1 and gently mix until contents are completely dissolved.
2) The final solution contains cAMP at a concentration of 512 pmol/mL.
3) Label 7 polypropylene or polystyrene tubes, 0.2 pmol, 0.4 pmol, 0.8 pmol, 1.6 pmol, 3.2 pmol, 6.4 pmol and 12.8 pmol.
4) Pipette 500 μL of lysis buffer 1 into all the tubes.
5) Into the 12.8 pmol tube pipette 500 μL of stock standard (512 pmol/mL) and mix thoroughly. Transfer 500 μL from 12.8 pmol tube to the 6.4 pmol tube and mix thoroughly. Repeat this doubling dilution successively with the remaining tubes.

6) 50 μL aliquots in duplicate from each serial dilution and the stock standard will give rise to 8 standard levels of cAMP ranging from 0.2-25.6 pmol standard

Compound Dilution Buffer

Add 50 μL of 1 mM IBMX into 100 mL PBS to make a final concentration of 100 μM and sonicate at 30° C. for 20 minutes.

PGD2 Preparation

Dissolve 1 mg PGD2 (FW, 352.5) in 284 μL DMSO to make 10 mM stock solution and store at 20° C. Before each assay, it is freshly prepared. Add 3 μL of 10 mM stock solution to 20 mL DMSO, mix it thoroughly, and transfer 10 mL to 40 mL PBS.

Compound Dilution

Compound dilution is carried out in Biomex 2000 (Beckman) using Method 1_cAMP DP 11 points.

5 μL of each compound from the 10 mM stock compound plates is transferred to the wells of a 96-well plate respectively as below.

| | 1 | 2 | 3 | 4 | 5 | 6 | 7 | 8 | 9 | 10 | 11 | 12 |
|---|---|---|---|---|---|---|---|---|---|---|---|---|
| A | 1 | | | | | | | | | | | |
| B | 2 | | | | | | | | | | | |
| C | 3 | | | | | | | | | | | |
| D | 4 | | | | | | | | | | | |
| E | 5 | | | | | | | | | | | |
| F | 6 | | | | | | | | | | | |
| G | 7 | | | | | | | | | | | |
| H | reference | | | | | | | | | | | |

Fill the plate with 45 μL of DMSO except column 7 is filled with 28 μL DMSO. Pipette column 1 thoroughly, and transfer 12 μL into column 7 parallel. Perform 1:10 serial dilution from column 1 to column 6 and from column 7 to column 11 by transfer 5 μL to 45 μL DMSO to make following concentrations:

| First plate | Final concentration | |
|---|---|---|
| Column 12 | 0 | |
| Column 11 | 0.03 | μM |
| Column 10 | 0.3 | μM |
| Column 9 | 3 | μM |
| Column 8 | 0.03 | mM |
| Column 7 | 0.3 | mM |
| Column 6 | 0.01 | μM |
| Column 5 | 0.1 | μM |
| Column 4 | 1 | μM |
| Column 3 | 0.01 | mM |
| Column 2 | 0.1 | mM |
| Column 1 | 1 | mM |

Fill a new 96-well plate with 247.5 μL of compound dilution buffer. Transfer 2.5 μL of serially diluted compounds from above plate to the new plate (1:100 dilution) as following:

| First plate | Second plate | Final concentration | |
|---|---|---|---|
| Column 12 | Column 1 | 0 | |
| Column 6 | Column 2 | 0.1 | nM |
| Column 11 | Column 3 | 0.3 | nM |
| Column 5 | Column 4 | 1 | nM |
| Column 10 | Column 5 | 3 | nM |
| Column 4 | Column 6 | 0.01 | μM |
| Column 9 | Column 7 | 0.03 | μM |
| Column 3 | Column 8 | 0.1 | μM |
| Column 8 | Column 9 | 0.3 | μM |
| Column 2 | Column 10 | 1 | μM |
| Column 7 | Column 11 | 3 | μM |
| Column 1 | Column 12 | 10 | μM |

Cell Growth
1. LS174 T are always grown in MEM (ATCC Cat# 30-2003), 10% FBS (ATCC Cat# 30-2020) and additional 2 mM L-glutamine, at 37° C. and 5% CO₂.
2. Warm 0.05% Trypsin and Versine (Invitrogen Cat# 25300-054) at 37° C. water bath.
3. Remove growth medium from cells. Cells in T165 flask are washed twice with 4 mL Trypsin followed by incubation at 37° C. and 5% CO₂ for 3 minutes.
4. Add 10 mL of medium and pipette thoroughly to separate the cells and count the cells.
5. Bring the cell density to 2.25×10⁵ cells/ml and seed 200 μL cells/well (45,000 cells/well) in 96-well plates 1 day before the assay.
   Assay Procedure

Day 1
Seed 45,000 cells/well in 200 μL medium in 96-well plates. Incubate the cell plate at 37° C., 5% CO₂ and 95% humidity overnight.

Day 2
1. Perform compound dilution.
2. Prepare assay buffer, lysis buffer 1 & 2, PGD₂ and standard.
3. Aspirate media from the cells and add 100 μL of compound solution using Zymark Sciclone-ALH/FD protocol cAMP DP.
4. Incubate the cells at 37° C., 5% CO₂ and 95% humidity for 15 minutes.
5. Add 5 μL of 300 nM PGD2 (20×15 nM final concentration) into each well using Zymark protocol cAMP DP PGD2, and incubate the cells at 37° C., 5% CO₂ and 95% humidity for additional 15 minutes.
6. Aspirate media from the cells and add 50 μL of lysis buffer 1 using Zymark protocol cAMP DP lysis, and incubate at room temperature with shaking for 30 minutes.
7. Add 150 μL immunoreagent to all wells (a total volume of 200 μL/well).
8. Seal the plates and shake for 2 minutes, put into the chamber of the Wallac microtitre plate μ scintillation counter for 16 hours.

Day 3
Count the amount of [¹²⁵I] cAMP for 2 minutes in 1450 Trilux scintillation counter.
   Data Processing

Set Up Standard Curve of Camp Versus CPM.

TABLE 1
Typical assay data for standard

| | | |
|---|---|---|
| cAMP | | |
| (pmol/mL) | CPM | Average CPM |

| | | | |
|---|---|---|---|
| 0.2 | 5725 | 5769 | 5530 |
| 0.4 | 5367 | 5259 | 6317 |
| 0.8 | 4695 | 4796 | 6507 |
| 1.6 | 4251 | 4178 | 6581 |
| 3.2 | 3434 | 3429 | 6601 |
| 6.4 | 2758 | 2716 | 6711 |
| 12.8 | 2094 | 2054 | 6680 |
| 25.6 | 1531 | 1573 | 6653 |

The cAMP concentrations (pmol/mL) of unknown samples are calculated from a standard curve of cAMP versus CPM. % inhibition is calculated using the following formula:

$$\% Inhibition = \frac{( pmol of control - pmol of sample ) \times 100}{pmol of control ( cells + PGD 2 only )}$$

Results

Compounds within the scope of the invention produce 50% inhibition in the SPA cAMP assay in human LS174 T cells at concentrations within the range of about 1 nanomolar to about 10 micromolar. Particular compounds within the scope of the invention produce 50% inhibition in the SPA cAMP assay in human LS174 T cells at concentrations within the range of about 1 to about 500 nanomolar. More particular compounds within the scope of the invention produce 50% inhibition in the SPA cAMP assay in human LS174 T cells at concentrations within the range of about 1 to about 100 nanomolar.

The present invention may be embodied in other specific forms without departing from the spirit or essential attributes thereof.

We claim:

1. A compound of Formula (XVI): 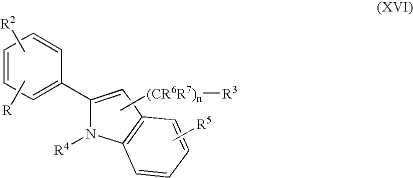
wherein:
R is R¹SO₂—, R¹SO—, R¹CO—, R⁸—C(═O)—NH—, or R⁸—SO₂—NH—;
R¹is alkyl, alkenyl, or alkynyl, each of which is optionally substituted by one or more aliphatic group substituents,
cycloalkyl, cycloalkenyl, aryl, heteroaryl, heterocyclyl, heterocyclenyl, or multicyclic alkaryl, each of which is optionally substituted by one or more ring group substituents, or
—NR′R″ when R is R¹SO₂— or R¹CO—;
R′ is hydrogen,
aryl, heteroaryl, cycloalkyl, cycloalkenyl, heterocyclyl, heterocyclenyl, or multicyclic alkaryl, each of which is optionally substituted by one or more ring group substituents, or
alkyl, alkenyl or alkynyl, each of which is optionally substituted by one or more aliphatic group substituents;
R″ is hydrogen, alkyl, alkenyl or alkynyl;
R² is hydrogen, halo, alkyl, alkenyl, alkynyl, haloalkyl, haloalkenyl, haloalkynyl, alkoxy, alkenyloxy or alkynyloxy;
R³ is acyl, cyano, carboxy, acid bioisostere, —C(O)—NY¹Y²,
aroyl or heteroaroyl, each of which is optionally substituted by one or more ring group substituents,
alkyl, or alkynyl, each of which is optionally substituted by one or more aliphatic group substituents, or
alkoxy, alkenyloxy or alkynyloxy, each of which is optionally substituted by one or more aliphatic group substituents;
Y¹ and Y² are each independently hydrogen, alkylsulfonyl, arylsulfonyl, arylamino, heteroarylsulfonyl, heteroarylamino, or
alkyl, alkenyl or alkynyl, each of which is optionally substituted by one or more aliphatic substituent groups;
R⁴ is hydrogen, acyl, aroyl, heteroaryl, alkylsulfonyl, arylsulfonyl, arylalkylsulfonyl, heteroarylsulfonyl, heteroarylalkylsulfonyl, —C(O)—NY⁴Y⁵, —C(O)—O—Y⁶,
alkyl, alkenyl or alkynyl, each of which is optionally substituted by aryl, heteroaryl, carboxy, alkoxycarbonyl, aminocarbonyl, alkylaminocarbonyl, dialkylaminocarbonyl, aroyl, heteroaroyl or acyl, or
(C₂-C₆)-alkyl, alkenyl or alkynyl, each of which is substituted by halo, hydroxy, alkoxy, amino, alkylamino or dialkylamino;
Y⁴ and Y⁵ are each independently hydrogen, alkyl, alkenyl or alkynyl;
Y⁶ is alkyl, alkenyl or alkynyl;
R⁵ is hydrogen, halo, carboxy, cyano, nitro, hydroxy, alkyl, alkenyl, alkynyl, haloalkyl, haloalkenyl, haloalkynyl, alkoxy, alkenyloxy, alkynyloxy, haloalkoxy, haloalkenyloxy or haloalkynyloxy;
R⁶ and R⁷ are each independently, hydrogen, alkyl, alkenyl or alkynyl;
R⁸ is alkyl, alkenyl, or alkynyl, each of which is optionally substituted by one or more aliphatic group substituents, or
aryl, heteroaryl, cycloalkyl, cycloalkenyl, heterocyclyl, heterocyclenyl, or multicyclic alkaryl, each of which is optionally substituted by one or more ring group substituents; and
n is 1 to 6, or 0 when R³ is carboxy, acid bioisostere, or —C(O)—NY¹Y²;
provided that when R¹ is amino, then R⁴ is hydrogen and n is 1 to 6;
or a pharmaceutically acceptable salt, hydrate, or solvate thereof, a pharmaceutically acceptable prodrug thereof, or a pharmaceutically acceptable salt, hydrate or solvate of the prodrug, provided that the prodrug is not 2-(4-acetyl-phenyl)-1H-indole-3-carboxylic acid methyl ester.

2. The compound according to claim 1, wherein n is 1 to 3, or 0 when R³ is carboxy, acid bioisostere, or —C(O)—NY¹Y², or a pharmaceutically acceptable salt, hydrate, or solvate thereof, a pharmaceutically acceptable prodrug thereof, or a pharmaceutically acceptable salt, hydrate or solvate of the prodrug.

3. The compound according to claim 1, wherein n is 1, or a pharmaceutically acceptable salt, hydrate, or solvate thereof, a pharmaceutically acceptable prodrug thereof, or a pharmaceutically acceptable salt, hydrate or solvate of the prodrug.

4. The compound according to claim 1, wherein the compound is of Formula (I): 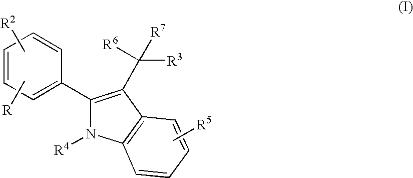
or a pharmaceutically acceptable salt, hydrate, or solvate thereof, a pharmaceutically acceptable prodrug thereof, or a pharmaceutically acceptable salt, hydrate or solvate of the prodrug.

5. The compound according to claim 4, wherein:
R is R¹SO₂—, R¹SO—, R⁸—C(═O)—NH— or R⁸—SO₂—NH—;
R¹is alkyl, alkenyl or alkynyl, each of which is optionally substituted by one or more aliphatic group substituents,
aryl, heteroaryl, or heterocyclyl, each of which is optionally substituted by one or more ring group substituents, or
—NR′R″ when R is R¹SO₂—;
R′ is hydrogen,
aryl, heteroaryl, cycloalkyl, heterocyclyl, arylcycloalkyl, or cycloalkylaryl, each of which is optionally substituted by one or more ring group substituents, or
alkyl, alkenyl or alknyl, each of which is optionally substituted by one or more aliphatic group substituents;
R″ is hydrogen, alkyl;
R² is hydrogen, halo, alkyl, alkenyl, alkynyl, haloalkyl, or alkoxy;
R³ is acyl, cyano, carboxy, acid bioisostere, —C(O)—NY¹Y²,
alkyl, which is optionally substituted by one or more aliphatic group substituents, or alkoxy, which is optionally substituted by one or more aliphatic group substituents,
Y¹ and Y² are each independently hydrogen, alkylsulfonyl, arylsulfonyl, arylamino, heteroarylsulfonyl, heteroarylamino, or
alkyl, which is optionally substituted by one or more aliphatic substituent groups;
R⁴ is hydrogen, acyl, alkylsulfonyl, arylsulfonyl, arylalkylsulfonyl, heteroarylsulfonyl, heteroarylalkylsulfonyl, —C(O)—NY⁴Y⁵, —C(O)—O—Y⁶,
alkyl, alkenyl or alkynyl, each of which is optionally substituted by carboxy, alkoxycarbonyl or acyl, or
(C₂-C₆)-alkyl, alkenyl or alkynyl, each of which is substituted by hydroxy, alkoxy, amino, alkylamino or dialkylamino;
Y⁴ and Y⁵ are each independently hydrogen, or alkyl;
Y⁶ is alkyl;
R⁵ is hydrogen, halo, carboxy, cyano, nitro, hydroxy, alkyl, alkenyl, alkynyl, haloalkyl, haloalkenyl, haloalkynyl, alkoxy, alkenyloxy, alkynyloxy, haloalkoxy, haloalkenyloxy or haloalkynyloxy;
R⁶ and R⁷ are each independently, hydrogen, or alkyl; and
R⁸ is alkyl, which is optionally substituted by one or more aliphatic group substituents, or
aryl, heteroaryl, cycloalkyl, heterocyclyl, arylcycloalkyl, cycloalkylaryl, heteroarylcycloalkyl, or cycloalkylheteroaryl, each of which is optionally substituted by one or more ring group substituents;
or a pharmaceutically acceptable salt, hydrate, or solvate thereof, a pharmaceutically acceptable prodrug thereof, or a pharmaceutically acceptable salt, hydrate or solvate of the prodrug.

6. The compound according to claim 4, wherein:
R is R¹SO₂—, R⁸—C(═O)—NH— or R⁸—SO₂—NH—;
R¹ is alkyl, aryl, arylalkyl, heteroaryl, heteroarylalkyl, heterocyclyl, or —NR′R″;
R′ is hydrogen, cycloalkyl, heterocyclyl, arylcycloalkyl, cycloalkylaryl, heteroarylcycloalkyl, cycloalkylheteroaryl,
aryl or heteroaryl, each of which is optionally substituted by alkyl, halo or haloalkyl, or
alkyl, which is optionally substituted by cycloalkyl, aryl, or heteroaryl, wherein the cycloalkyl, aryl or heteroaryl is optionally substituted by alkyl, halo or haloalkyl;
R″ is hydrogen or alkyl;
R² is hydrogen, halo, alkyl, haloalkyl or alkoxy;
R³ is acyl, cyano, carboxy, acid bioisostere, —C(O)—NY¹Y²,
alkyl, which optionally substituted by hydroxy, alkoxy, amino, alkylamino or dialkylamino, or alkoxy, which is optionally substituted by hydroxy, alkoxy, amino, alkylamino or dialkylamino,
Y¹ and Y² are each independently hydrogen, alkylsulfonyl, arylsulfonyl, heteroarylsulfonyl, arylamino, heteroarylamino, or
alkyl, which is optionally substituted by carboxy or alkoxycarbonyl;
R⁴ is hydrogen, acyl, alkylsulfonyl, arylsulfonyl, arylalkylsulfonyl, heteroarylalkyl, heteroarylsulfonyl, heteroarylalkylsulfonyl, arylalkyl, —C(O)—NY⁴Y⁵, —C(O)—O—Y⁶, alkyl, which is optionally substituted by carboxy, alkoxycarbonyl or acyl, or
(C₂-C₆)-alkyl, which is substituted by hydroxy, alkoxy, amino, alkylamino or dialkylamino;
Y⁴ and Y⁵ are each independently hydrogen or alkyl;
Y⁶ is alkyl;
R⁵ is hydrogen, halo, carboxy, cyano, nitro, hydroxy, alkyl, haloalkyl, alkoxy or haloalkoxy;
R⁶ and R⁷ are each independently, hydrogen or alkyl; and
R⁸ is alkyl, aryl, heteroaryl, cycloalkyl, heterocyclyl, arylcycloalkyl, or cycloalkylaryl;
or a pharmaceutically acceptable salt, hydrate, or solvate thereof, a pharmaceutically acceptable prodrug thereof, or a pharmaceutically acceptable salt, hydrate or solvate of the prodrug.

7. The compound according to claim 4, wherein:
R is R¹SO₂—, R⁸—C(═O)—NH— or R⁸—SO₂—NH—;
R¹ is alkyl, aryl, arylalkyl, heterocyclyl or —NR′R″;
R′ is hydrogen, cycloalkyl, heterocyclyl, arylcycloalkyl, cycloalkylaryl,
aryl, which is optionally substituted by alkyl, halo or haloalkyl, or
alkyl, which is optionally substituted by cycloalkyl or aryl, wherein the aryl is optionally substituted by alkyl, halo or haloalkyl;
R″ is hydrogen or alkyl;
R² is hydrogen, halo, alkyl, haloalkyl or alkoxy;
R³ is acyl, cyano, carboxy, acid bioisostere, —C(O)—NY¹Y²,
alkyl, which optionally substituted by hydroxy, alkoxy, amino, alkylamino or dialkylamino, or alkoxy, which is optionally substituted by hydroxy, alkoxy, amino, alkylamino or dialkylamino,
Y¹ and Y² are each independently hydrogen, alkylsulfonyl, arylsulfonyl, arylamino, or alkyl, which is optionally substituted by carboxy or alkoxycarbonyl;
R⁴ is hydrogen, acyl, alkylsulfonyl, arylsulfonyl, arylalkylsulfonyl, arylalkyl, —C(O)—NY⁴Y⁵, —C(O)—O—Y⁶,
alkyl, which is optionally substituted by carboxy, alkoxycarbonyl or acyl, or
(C₂-C₆)-alkyl, which is substituted by hydroxy, alkoxy, amino, alkylamino or dialkylamino;
Y⁴ and Y⁵ are each independently hydrogen or alkyl;
Y⁶ is alkyl;
R⁵ is hydrogen, halo, carboxy, cyano, nitro, hydroxy, alkyl, haloalkyl, alkoxy or haloalkoxy;
R⁶ and R⁷ are each independently, hydrogen or alkyl; and
R⁸ is aryl, cycloalkyl, heterocyclyl, arylcycloalkyl, or cycloalkylaryl;
or a pharmaceutically acceptable salt, hydrate, or solvate thereof, a pharmaceutically acceptable prodrug thereof, or a pharmaceutically acceptable salt, hydrate or solvate of the prodrug.

8. The compound according to claim 4, wherein R is R¹SO₂—, or a pharmaceutically acceptable salt, hydrate, or solvate thereof, a pharmaceutically acceptable prodrug thereof, or a pharmaceutically acceptable salt, hydrate or solvate of the prodrug.

9. The compound according to claim 4, wherein R is R¹SO₂—, and R¹ is —NR′R″, or a pharmaceutically acceptable salt, hydrate, or solvate thereof, a pharmaceutically acceptable prodrug thereof, or a pharmaceutically acceptable salt, hydrate or solvate of the prodrug.

10. The compound according to claim 4, wherein
R is R¹SO₂—;
R¹ is —NR′R″;
R′ is cycloalkyl, heterocyclyl, arylcycloalkyl or cycloalkylaryl; and
R″ is hydrogen or alkyl;
or a pharmaceutically acceptable salt, hydrate, or solvate thereof, a pharmaceutically acceptable prodrug thereof, or a pharmaceutically acceptable salt, hydrate or solvate of the prodrug.

11. The compound according to claim 4, wherein R is R¹SO₂—, R¹ is —NR′R″, R′ is cycloalkyl, and R″ is hydrogen or alkyl, or a pharmaceutically acceptable salt, hydrate, or solvate thereof, a pharmaceutically acceptable prodrug thereof, or a pharmaceutically acceptable salt, hydrate or solvate of the prodrug.

12. The compound according to claim 4, wherein R is R⁸—SO₂—NH—, or a pharmaceutically acceptable salt, hydrate, or solvate thereof, a pharmaceutically acceptable prodrug thereof, or a pharmaceutically acceptable salt, hydrate or solvate of the prodrug.

13. The compound according to claim 4, wherein R² is halo, or a pharmaceutically acceptable salt, hydrate, or solvate thereof, a pharmaceutically acceptable prodrug thereof, or a pharmaceutically acceptable salt, hydrate or solvate of the prodrug.

14. The compound according to claim 4, wherein R² is chloro, or a pharmaceutically acceptable salt, hydrate, or solvate thereof, a pharmaceutically acceptable prodrug thereof, or a pharmaceutically acceptable salt, hydrate or solvate of the prodrug.

15. The compound according to claim 4, wherein R² is alkyl, alkoxy or haloalkyl, or a pharmaceutically acceptable salt, hydrate, or solvate thereof, a pharmaceutically acceptable prodrug thereof, or a pharmaceutically acceptable salt, hydrate or solvate of the prodrug.

16. The compound according to claim 4, wherein R² is methyl, methoxy or —CF₃, or a pharmaceutically acceptable salt, hydrate, or solvate thereof, a pharmaceutically acceptable prodrug thereof, or a pharmaceutically acceptable salt, hydrate or solvate of the prodrug.

17. The compound according to claim 4, wherein R³ is —C(O)—NY¹Y², carboxy, acid bioisostere; or alkyl substituted by hydroxy; or a pharmaceutically acceptable salt, hydrate, or solvate thereof, a pharmaceutically acceptable prodrug thereof, or a pharmaceutically acceptable salt, hydrate or solvate of the prodrug.

18. The compound according to claim 4, wherein R³ is —COOH, or a pharmaceutically acceptable salt, hydrate, or solvate thereof, a pharmaceutically acceptable prodrug thereof, or a pharmaceutically acceptable salt, hydrate or solvate of the prodrug.

19. The compound according to claim 4, wherein R⁴ is hydrogen, alkyl or arylalkyl, or a pharmaceutically acceptable salt, hydrate, or solvate thereof, a pharmaceutically acceptable prodrug thereof, or a pharmaceutically acceptable salt, hydrate or solvate of the prodrug.

20. The compound according to claim 4, wherein R⁴ is hydrogen, or a pharmaceutically acceptable salt, hydrate, or solvate thereof, a pharmaceutically acceptable prodrug thereof, or a pharmaceutically acceptable salt, hydrate or solvate of the prodrug.

21. The compound according to claim 4, wherein R⁵ is hydrogen, alkyl, alkoxy, hydroxy, halo or haloalkoxy, or a pharmaceutically acceptable salt, hydrate, or solvate thereof, a pharmaceutically acceptable prodrug thereof, or a pharmaceutically acceptable salt, hydrate or solvate of the prodrug.

22. The compound according to claim 4, wherein R⁶ and R⁷ are both hydrogen, or a pharmaceutically acceptable salt, hydrate, or solvate thereof, a pharmaceutically acceptable prodrug thereof, or a pharmaceutically acceptable salt, hydrate or solvate of the prodrug.

23. The compound according to claim 4, wherein:
R is R¹SO₂—;
R¹ is —NR′R″;
R² is halo;
R³ is —C(O)—NY¹Y², carboxy, acid bioisostere; or alkyl substituted by hydroxy;
R⁴ is hydrogen, alkyl or arylalkyl;
R⁵ is hydrogen, alkyl, alkoxy, hydroxy, halo or haloalkoxy; and
R⁶ and R⁷ are both hydrogen;
or a pharmaceutically acceptable salt, hydrate, or solvate thereof, a pharmaceutically acceptable prodrug thereof, or a pharmaceutically acceptable salt, hydrate or solvate of the prodrug.

24. The compound according to claim 4, wherein:
R is R¹SO₂—;
R¹ is —NR′R″;
R′ is cycloalkyl, heterocyclyl, arylcycloalkyl, cycloalkylaryl, or
alkyl, which is optionally substituted by cycloalkyl or aryl, wherein the aryl is optionally substituted by haloalkyl;
R″ is hydrogen or alkyl;
R² is halo;
R³ is —C(O)—NY¹Y², carboxy, or acid bioisostere;
Y¹ and Y² are each independently hydrogen, alkylsulfonyl, arylsulfonyl, or alkyl substituted by carboxy or alkoxycarbonyl;
R⁴ is hydrogen, alkyl or arylalkyl;
R⁵ is hydrogen, alkyl, alkoxy, hydroxy, halo or haloalkoxy; and
R⁶ and R⁷ are both hydrogen;
or a pharmaceutically acceptable salt, hydrate, or solvate thereof, a pharmaceutically acceptable prodrug thereof, or a pharmaceutically acceptable salt, hydrate or solvate of the prodrug.

25. The compound according to claim 4, wherein:
R is R¹SO₂—;
R¹ is piperidinyl or —NR′R″;
R′ is hydrogen, cycloheptane, cycloheptane-methylene, cyclohexane, cyclohexane-methylene, cyclohexane-ethylene, cyclopentane, bicyclo[2.2.1]heptane, indanyl, phenyl, tetrahydropyranyl, tricyclo[3.3.1.13.7]decane-methylene, methyl, isopropyl, isopentyl, n-hexanyl, benzyl, or 4-trifluoromethyl-benzyl;
R″ is hydrogen or methyl;
R² is chloro;
R³ is carboxy, —CH₂—OH, —C(O)—NH₂, —C(═O)—NH—SO₂—CH₃, 5-oxo-4,5-dihydro-1,3,4-oxadiazol-2-yl, 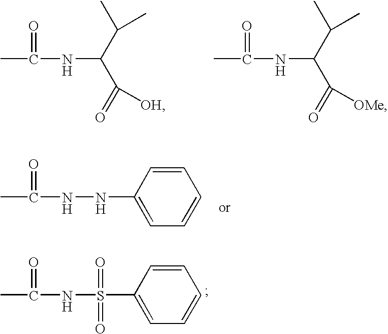
R⁴ is hydrogen, methyl or benzyl;
R⁵ is hydrogen, chloro, hydroxy, methyl, isopropyl, t-butyl, methoxy or trifluoromethoxy; and
R⁶ and R⁷ are both hydrogen;
or a pharmaceutically acceptable salt, hydrate, or solvate thereof, a pharmaceutically acceptable prodrug thereof, or a pharmaceutically acceptable salt, hydrate or solvate of the prodrug.

26. The compound according to claim 4, wherein:
R is R¹SO₂—;
R¹ is —NR′R″;
R′ is cycloheptane, cycloheptane-methylene, cyclohexane, cyclohexane-methylene, cyclohexane-ethylene, cyclopentane, bicyclo[2.2.1]heptane, indanyl, tetrahydropyranyl,
tricyclo[3.3.1.13.7]decane-methylene, isopropyl, isopentyl, n-hexanyl, benzyl, or 4-trifluoromethyl-benzyl;
R″ is hydrogen or methyl;
R² is chloro;
R³ is carboxy, —C(O)—NH₂, 5-oxo-4,5-dihydro-1,3,4-oxadiazol-2-yl, 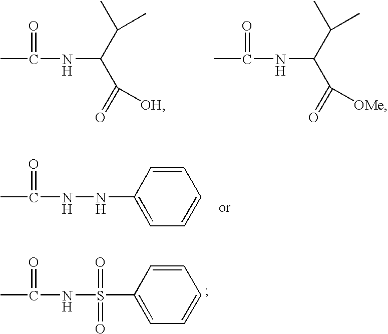
R⁴ is hydrogen, methyl or benzyl;
R⁵ is hydrogen, chloro, hydroxy, methyl, isopropyl, t-butyl, methoxy or trifluoromethoxy; and
R⁶ and R⁷ are both hydrogen;
or a pharmaceutically acceptable salt, hydrate, or solvate thereof, a pharmaceutically acceptable prodrug thereof, or a pharmaceutically acceptable salt, hydrate or solvate of the prodrug.

27. The compound according to claim 4, wherein the compound is of Formula (II): 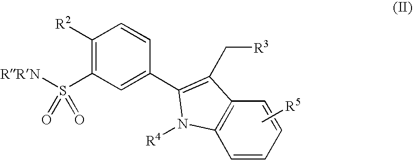
or a pharmaceutically acceptable salt, hydrate, or solvate thereof, a pharmaceutically acceptable prodrug thereof, or a pharmaceutically acceptable salt, hydrate or solvate of the prodrug.

28. The compound according to claim 27, wherein R′ is cycloalkyl, heterocyclyl, arylcycloalkyl or cycloalkylaryl, and R″ is hydrogen or alkyl; or a pharmaceutically acceptable salt, hydrate, or solvate thereof, a pharmaceutically acceptable prodrug thereof, or a pharmaceutically acceptable salt, hydrate or solvate of the prodrug.

29. The compound according to claim 27, wherein R′ is cycloalkyl, and R″ is hydrogen or alkyl;
or a pharmaceutically acceptable salt, hydrate, or solvate thereof, a pharmaceutically acceptable prodrug thereof, or a pharmaceutically acceptable salt, hydrate or solvate of the prodrug.

30. The compound according to claim 27, wherein R² is halo, or a pharmaceutically acceptable salt, hydrate, or solvate thereof, a pharmaceutically acceptable prodrug thereof, or a pharmaceutically acceptable salt, hydrate or solvate of the prodrug.

31. The compound according to claim 27, wherein R² is chloro, or a pharmaceutically acceptable salt, hydrate, or solvate thereof, a pharmaceutically acceptable prodrug thereof, or a pharmaceutically acceptable salt, hydrate or solvate of the prodrug.

32. The compound according to claim 27, wherein R² is alkyl, alkoxy or haloalkyl, or a pharmaceutically acceptable salt, hydrate, or solvate thereof, a pharmaceutically acceptable prodrug thereof, or a pharmaceutically acceptable salt, hydrate or solvate of the prodrug.

33. The compound according to claim 27, wherein R² is methyl, methoxy or —CF₃, or a pharmaceutically acceptable salt, hydrate, or solvate thereof, a pharmaceutically acceptable prodrug thereof, or a pharmaceutically acceptable salt, hydrate or solvate of the prodrug.

34. The compound according to claim 27, wherein R³ is —C(O)—NY¹Y², carboxy, acid bioisostere; alkyl substituted by hydroxy; or a pharmaceutically acceptable salt, hydrate, or solvate thereof, a pharmaceutically acceptable prodrug thereof, or a pharmaceutically acceptable salt, hydrate or solvate of the prodrug.

35. The compound according to claim 27, wherein R³ is —COOH, or a pharmaceutically acceptable salt, hydrate, or solvate thereof, a pharmaceutically acceptable prodrug thereof, or a pharmaceutically acceptable salt, hydrate or solvate of the prodrug.

36. The compound according to claim 27, wherein R⁴ is hydrogen, alkyl or arylalkyl, or a pharmaceutically acceptable salt, hydrate, or solvate thereof, a pharmaceutically acceptable prodrug thereof, or a pharmaceutically acceptable salt, hydrate or solvate of the prodrug.

37. The compound according to claim 27, wherein R⁴ is hydrogen, or a pharmaceutically acceptable salt, hydrate, or solvate thereof, a pharmaceutically acceptable prodrug thereof, or a pharmaceutically acceptable salt, hydrate or solvate of the prodrug.

38. The compound according to claim 27, wherein R⁵ is hydrogen, alkyl, alkoxy, hydroxy, halo or haloalkoxy, or a pharmaceutically acceptable salt, hydrate, or solvate thereof, a pharmaceutically acceptable prodrug thereof, or a pharmaceutically acceptable salt, hydrate or solvate of the prodrug.

39. The compound according to claim 27, wherein:
R′ is cycloalkyl, heterocyclyl, arylcycloalkyl, cycloalkylaryl, or
alkyl, optionally substituted by cycloalkyl or aryl, wherein the aryl is optionally substituted by haloalkyl;
R″ is hydrogen or alkyl;
R² is halo;
R³ is —C(O)—NY¹Y², carboxy, acid bioisostere; alkyl substituted by hydroxy;
R⁴ is hydrogen, alkyl or arylalkyl; and
R⁵ is hydrogen, alkyl, alkoxy, hydroxy, halo or haloalkoxy,
or a pharmaceutically acceptable salt, hydrate, or solvate thereof, a pharmaceutically acceptable prodrug thereof, or a pharmaceutically acceptable salt, hydrate or solvate of the prodrug.

40. The compound according to claim 27, wherein:
R′ is cycloalkyl, heterocyclyl, arylcycloalkyl or cycloalkylaryl, or alkyl or alkyl substituted by cycloalkyl;
R″ is hydrogen or alkyl;
R² is halo;
R³ is —C(O)_NY¹Y², carboxy, or acid bioisostere;
Y¹ and Y² are each independently hydrogen, alkylsulfonyl, arylsulfonyl, or alkyl substituted by carboxy or alkoxycarbonyl;
R⁴ is hydrogen, alkyl or arylalkyl; and
R⁵ is hydrogen, alkyl, alkoxy, hydroxy, halo or haloalkoxy,
or a pharmaceutically acceptable salt, hydrate, or solvate thereof, a pharmaceutically acceptable prodrug thereof, or a pharmaceutically acceptable salt, hydrate or solvate of the prodrug.

41. The compound according to claim 27, wherein:
R′ is hydrogen, cycloheptane, cycloheptane-methylene, cyclohexane, cyclohexane-methylene, cyclohexane-ethylene, cyclopentane, bicyclo[2.2.1]heptane, indanyl, phenyl, tetrahydropyranyl, tricyclo[3.3.1.13.7]decane-methylene, methyl, isopropyl, isopentyl, n-hexanyl, benzyl or 4-trifluoromethyl-benzyl;
R″ is hydrogen or methyl;
R² is chloro;
R³ is carboxy, —CH₂—OH, —C(O)—NH₂, —C(═O)—NH—SO₂—CH₃, 5-oxo-4,5-dihydro-1,3,4-oxadiazol-2-yl, 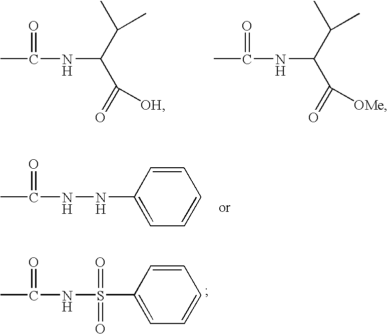
R⁴ is hydrogen, methyl or benzyl; and
R⁵ is hydrogen, chloro, hydroxy, methyl, isopropyl, t-butyl, methoxy or trifluoromethoxy,
or a pharmaceutically acceptable salt, hydrate, or solvate thereof, a pharmaceutically acceptable prodrug thereof, or a pharmaceutically acceptable salt, hydrate or solvate of the prodrug.

42. The compound according to claim 27, wherein
R′ is cycloheptane, cycloheptane-methylene, cyclohexane, cyclohexane-methylene, cyclohexane-ethylene, cyclopentane, bicyclo[2.2.1]heptane, indanyl, tetrahydropyranyl, tricyclo[3.3.1.13.7]decane-methylene, isopropyl, isopentyl, n-hexanyl, benzyl or 4-trifluoromethyl-benzyl;
R″ is hydrogen or methyl;
R² is chloro;
R³ is carboxy, —C(O)—NH₂, 5-oxo-4,5-dihydro-1,3,4-oxadiazol-2-yl, 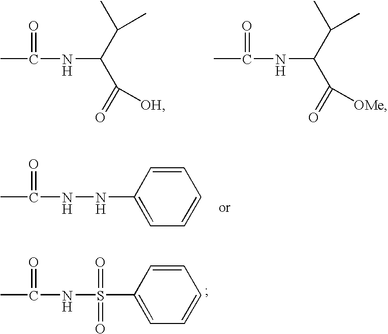
R⁴ is hydrogen, methyl or benzyl; and
R⁵ is hydrogen, chloro, hydroxy, methyl, isopropyl, t-butyl, methoxy or trifluoromethoxy,
or a pharmaceutically acceptable salt, hydrate, or solvate thereof, a pharmaceutically acceptable prodrug thereof, or a pharmaceutically acceptable salt, hydrate or solvate of the prodrug.

43. The compound or the pharmaceutically acceptable ester prodrug according to claim 1, which is:
[2-(4-Chloro-3-cyclohexylsulfamoyl-phenyl)-1H-indol-3-yl]-acetic acid;
{2-[3-(Bicyclo[2.2.1]hept-2-ylsulfamoyl)-4-chloro-phenyl]-1H-indol-3-yl}-acetic acid;
[2-(4-Chloro-3-hexylsulfamoyl-phenyl)-1H-indol-3-yl]-acetic acid;
{2-[4-Chloro-3-(indan-2-ylsulfamoyl)-phenyl]-1H-indol-3-yl}-acetic acid;
[2-(4-Chloro-3-cyclopentylsulfamoyl-phenyl)-1H-indol-3-yl]-acetic acid;
{2-[4-Chloro-3-(2,2-dimethyl-propylsulfamoyl)-phenyl]-1H-indol-3-yl}-acetic acid;
[2-(4-Chloro-3-isopropylsulfamoyl-phenyl)-1H-indol-3-yl]-acetic acid;
{2-[4-Chloro-3-(2-cyclohexyl-ethylsulfamoyl)-phenyl]-1H-indol-3-yl}-acetic acid;
[2-(4-Chloro-3-phenylsulfamoyl-phenyl)-1H-indol-3-yl]-acetic acid;
{2-[4-Chloro-3-(cyclohexylmethyl-sulfamoyl)-phenyl]-1H-indol-3-yl}-acetic acid;
{2-[4-Chloro-3-(1-ethyl-propylsulfamoyl)-phenyl]-1H-indol-3-yl}-acetic acid;
{2-[4-Chloro-3-(cycloheptylmethyl-sulfamoyl)-phenyl]-1H-indol-3-yl}-acetic acid;
(2-{4-Chloro-3-[(tricyclo[3.3.1.13.7]decan-1-ylmethyl)-sulfamoyl]-phenyl}-1H-indol-3-yl)-acetic acid;
[2-(4-Chloro-3-cycloheptylsulfamoyl-phenyl)-1H-indol-3-yl]-acetic acid;
{2-[4-Chloro-3-(tetrahydro-pyran-4-ylsulfamoyl)-phenyl]-1H-indol-3-yl}-acetic acid;
{2-[4-Chloro-3-(piperidine-1-sulfonyl)-phenyl]-1H-indol-3-yl}-acetic acid;
[2-(4-Chloro-3-methylsulfamoyl-phenyl)-1H-indol-3-yl]-acetic acid;
[2-(4-Chloro-3-sulfamoyl-phenyl)-1H-indol-3-yl]-acetic acid;
[5-tert-Butyl-2-(4-chloro-3-cyclohexylsulfamoyl-phenyl)-1H-indol-3-yl]-acetic acid;
[2-(4-Chloro-3-cyclohexylsulfamoyl-phenyl)-5-methyl-1H-indol-3-yl]-acetic acid;
[2-(4-Chloro-3-cyclohexylsulfamoyl-phenyl)-5-isopropyl-1H-indol-3-yl]-acetic acid;
[2-(4-Chloro-3-cyclohexylsulfamoyl-phenyl)-5-trifluoromethoxy-1H-indol-3-yl]-acetic acid;
[2-(3-Benzylsulfamoyl-4-chloro-phenyl)-1H-indol-3-yl]-acetic acid;
{2-[4-Chloro-3-(cyclohexyl-methyl-sulfamoyl)-phenyl]-1H-indol-3-yl}-acetic acid;
{2-[4-Chloro-3-(4-trifluoromethyl-benzylsulfamoyl)-phenyl]-1H-indol-3-yl}-acetic acid;
[2-(4-Chloro-3-cyclohexylsulfamoyl-phenyl)-1-methyl-1H-indol-3-yl]-acetic acid;
[1-Benzyl-2-(4-chloro-3-cyclohexylsulfamoyl-phenyl)-1H-indol-3-yl]-acetic acid;
{2-[4-Chloro-3-(piperidine-1-sulfonyl)-phenyl]-1-methyl-1H-indol-3-yl}-acetic acid;
(S)-2-{2-[2-(4-Chloro-3-cyclohexylsulfamoyl-phenyl)-1H-indol-3-yl]-acetylamino}-3-methyl-butyric acid;
(S)-2-{2-[2-(4-Chloro-3-cyclohexylsulfamoyl-phenyl)-1H-indol-3-yl]-acetylamino}-3-methyl-butyric acid;
[2-(4-Chloro-3-cyclohexylsulfamoyl-phenyl)-1H-indol-3-yl]-acetic acid 2-dimethylamino-ethyl ester;
2-Chloro-N-cyclohexyl-5-[3-(5-oxo-4,5-dihydro-1,3,4-oxadiazol-2-ylmethyl)-1H-indol-2-yl]-benzenesulfonamide;
5-[3-(2-Benzenesulfonylamino-2-oxo-ethyl)-1H-indol-2-yl]-2-chloro-N-cyclohexyl benzenesulfonamide;
2-[2-(4-Chloro-3-cyclohexylsulfamoyl-phenyl)-1H-indol-3-yl]-acetamide;
2-[2-(4-Chloro-3-cyclohexylsulfamoyl-phenyl)-1-methyl-1H-indol-3-yl]-acetamide;
[2-(4-Chloro-3-cyclohexylsulfamoyl-phenyl)-1H-indol-3-yl]-acetic acid methyl ester;
2-Chloro-N-cyclohexyl-5-[3-(2-hydroxy-ethyl)-1-methyl-1H-indol-2-yl]-benzenesulfonamide;
[2-(4-Chloro-3-cyclohexylsulfamoyl-phenyl)-5-methoxy-1H-indol-3-yl]-acetic acid;
[5-Chloro-2-(4-chloro-3-cyclohexylsulfamoyl-phenyl)-1H-indol-3-yl]-acetic acid;
[2-(4-Chloro-3-cyclohexylsulfamoyl-phenyl)-5-hydroxy-1H-indol-3-yl]-acetic acid;
[6-Chloro-2-(4-chloro-3-cyclohexylsulfamoyl-phenyl)-1H-indol-3-yl]-acetic acid;
{2-[3-(Cyclohexyl-methyl-sulfamoyl)-phenyl]-1H-indol-3-yl}-acetic acid;
[2-(3-Cyclohexylsulfamoyl-phenyl)-1H-indol-3-yl]-acetic acid;
2-[2-(3-Cyclohexylsulfamoyl-phenyl)-1H-indol-3-yl]-propionic acid;
[2-(4-Cyclohexylsulfamoyl-phenyl)-1H-indol-3-yl]-acetic acid;
-[2-(3-Cyclohexylsulfamoyl-4-methoxy-phenyl)-1H-indol-3-yl]-acetic acid;
[2-(3-Chloro-4-cyclohexylsulfamoyl-phenyl)-1H-indol-3-yl]-acetic acid;
[2-(3-Cyclohexylsulfamoyl-4-methyl-phenyl)-1H-indol-3-yl]-acetic acid;
[2-(3-Cyclohexylsulfamoyl-5-trifluoromethyl-phenyl)-1H-indol-3-yl]-acetic acid methyl ester;
[2-(3-Cyclohexylsulfamoyl-5-trifluoromethyl-phenyl)-1H-indol-3-yl]-acetic acid;
[2-(3-Benzenesulfonylamino-4-chlorophenyl)-1H-indol-3-yl]-acetic acid;
{2-[4-Chloro-3-(cyclohexanecarbonyl-amino)-phenyl]-1H-indol-3-yl}-acetic acid;
2-(4-Chloro-3-cyclohexylsulfamoyl-phenyl)-1H-indole-3-carboxylic acid; or
2-(4-Chloro-3-cyclohexylsulfamoyl-phenyl)-1H-indole-6-carboxylic acid;
or a pharmaceutically acceptable salt, hydrate, or solvate thereof.

44. A pharmaceutical composition comprising a pharmaceutically effective amount of the compound according to claim 1, or a pharmaceutically acceptable salt, hydrate, or solvate thereof, a pharmaceutically acceptable prodrug thereof, or a pharmaceutically acceptable salt, hydrate or solvate of the prodrug, in admixture with a pharmaceutically acceptable carrier.

45. A pharmaceutical composition comprising a pharmaceutically effective amount of the compound according to claim 4, or a pharmaceutically acceptable salt, hydrate, or solvate thereof, a pharmaceutically acceptable prodrug thereof, or a pharmaceutically acceptable salt, hydrate or solvate of the prodrug, in admixture with a pharmaceutically acceptable carrier.

46. A method for treating an allergic disease, systemic mastocytosis, a disorder accompanied by systemic mast cell activation, anaphylaxis shock, bronchoconstriction, bronchitis, urticaria, eczema, a diseases accompanied by itch, a disease which is generated secondarily as a result of behavior accompanied by itch, inflammation, chronic obstructive pulmonary diseases, ischemic reperfusion injury, cerebrovascular accident, chronic rheumatoid arthritis, pleurisy, or ulcerative colitis, in a patient in need thereof, comprising administering to the patient a pharmaceutically effective amount of the compound according to claim 1, or a pharmaceutically acceptable salt, hydrate, or solvate thereof, a pharmaceutically acceptable prodrug thereof, or a pharmaceutically acceptable salt, hydrate or solvate of the prodrug.

47. The method according to claim 46, wherein the a disease which is generated secondarily as a result of behavior accompanied by itch is cataract, retinal detachment, inflammation, infection or sleeping disorder.

48. The method according to claim 46 for treating the allergic disease or chronic obstructive pulmonary disease.

49. The method according to claim 48, wherein the allergic disease is an allergic disease, bronchial asthma, allergic rhinitis, allergic dermatitis, allergic conjunctivitis.

50. A pharmaceutical composition comprising a pharmaceutically effective amount of a compound according to claim 1, a compound selected from the group consisting of an antihistamine, a leukotriene antagonist, a beta agonist, a PDE4 inhibitor, a TP antagonist and a CrTh2 antagonist, in admixture with a pharmaceutically acceptable carrier.

51. The pharmaceutical composition according to claim 50, wherein the antihistamine is fexofenadine, loratadine or citirizine, the leukotriene antagonist is montelukast or zafirlukast, the beta agonist is albuterol, salbuterol or terbutaline, the PDE4 inhibitor is roflumilast or cilomilast, the TP antagonist is Ramatroban, and the CrTh2 antagonist is Ramatroban.
